SEQUENCE LISTING (11 sequences)

SEQ ID NO: 1 - RNA, 21 nt, Artificial Sequence
  synthesized siRNA sequences - oligo-1 (sense sequence)
gcuuaccgucagauacaaguu

SEQ ID NO: 2 - RNA, 21 nt, Artificial Sequence
  synthesized siRNA sequences - oligo-1 (antisense sequence)
cuuguaucugacgguaagcuu

SEQ ID NO: 3 - RNA, 21 nt, Artificial Sequence
  synthesized siRNA sequences - oligo-2 (sense sequence)
gacggcuaaguccuuguuuuu

SEQ ID NO: 4 - RNA, 21 nt, Artificial Sequence
  synthesized siRNA sequences - oligo-2 (antisense sequence)
aaacaaggacuuagccgucuu

SEQ ID NO: 5 - RNA, 21 nt, Artificial Sequence
  synthesized siRNA sequences - oligo-3 (sense sequence)
acacuugagggccuguauguu

SEQ ID NO: 6 - RNA, 21 nt, Artificial Sequence
  synthesized siRNA sequences - oligo-3 (antisense sequence)
cauacaggcccucaaguguuu

SEQ ID NO: 7 - RNA, 21 nt, Artificial Sequence
  synthesized siRNA sequences - oligo-4 (sense sequence)
ccugcucucggaggaugccuu

SEQ ID NO: 8 - RNA, 21 nt, Artificial Sequence
  synthesized siRNA sequences - oligo-4 (antisense sequence)
ggcauccuccgagagcagguu

SEQ ID NO: 9 - DNA, 19 bp, Artificial Sequence
  synthesized forward primer for real-time RT-PCR
atgcctttggccattgttg

SEQ ID NO: 10 - DNA, 23 bp, Artificial Sequence
  synthesized reverse primer for real-time RT-PCR
acaatccacttctacagctgcct

SEQ ID NO: 11 - DNA, 14 bp, Artificial Sequence
  synthesized A3 MGB probe
tcagcctgggcatc

(19) United States
(12) Patent Application Publication
(10) Pub. No.: US 2006/0204502 A1
(43) Pub. Date: Sep. 14, 2006

(54) Enhancing treatment of cancer and HIF-1 mediated disorders with adenosine A3 receptor antagonists

(76) Inventors: Pier Andrea Borea, Ferrara (IT); Pier Giovanni Baraldi, Ferrara (IT); Stefania Merighi, Ferrara (IT); Stephen MacLennan, Cary, NC (US); Edward Leung, Cary, NC (US); Allan Moorman, Durham, NC (US)

Correspondence Address: JONES DAY, 222 EAST 41ST ST, NEW YORK, NY, 10017, US

(21) Appl. No.: 11/286,127

(22) Filed: Nov. 22, 2005

Related U.S. Application Data
(60) Provisional application No. 60/630,555, filed on Nov. 22, 2004.

(51) Int. Cl. A61K 39/395; A61K 31/519

(52) U.S. Cl. 424/155.1; 514/267

(57) ABSTRACT

The present invention relates to the use of adenosine receptor antagonists, preferably A₃ receptor antagonists, either alone or in combination with other agents for the treatment, prevention and/or management of diseases or disorders associated with overexpression of HIF-1α and/or increased HIF-1α activity (e.g., cancer, respiratory disease). The methods and compositions of the invention are particularly useful for preventing, treating, or ameliorating symptoms associated with a cancer, disease or disorder associated with hypoxia-inducible factor 1α (HIF-1α) using the A₃ receptor antagonists of the invention. The present invention provides methods to inhibit the growth of tumors, particularly solid tumors and more particularly hypoxic tumors.

### 1. CROSS-REFERENCE TO RELATED APPLICATIONS

This application claims the benefit of U.S. Provisional Application No. 60/630,557, filed Nov. 22, 2004, the disclosure of which is hereby incorporated by reference in its entirety.

### 2. FIELD OF THE INVENTION

The present invention relates to the use of adenosine receptor antagonists, preferably A₃ receptor antagonists, either alone or in combination with other agents for the treatment, prevention and/or management of diseases or disorders associated with overexpression of HIF-1α and/or increased HIF-1α activity (e.g., cancer, respiratory disease). The methods and compositions of the invention are particularly useful for preventing, treating, or ameliorating symptoms associated with a cancer, disease or disorder associated with hypoxia-inducible factor 1-α (HIF-1α) using the A₃ receptor antagonists of the invention. The present invention provides methods to inhibit the growth of tumors, particularly solid tumors, and more particularly hypoxic tumors.

### 3. BACKGROUND OF THE INVENTION

3.1 Adenosine

Adenosine, recently called a “primordial signalling molecule” (Linden, 2001, Annu. Rev. Pharmacol. Toxicol, Unital 41: 775-87), has the potential of influencing development, is present in and modulates physiological responses in all mammalian tissues. The actions of adenosine are most prominent in tissues where oxygen demand is high and there is reduction in oxygen tension, i.e., within solid tumors, where cell proliferation is greater than the rate of blood vessel formation (Sitkovsky, 2004 Annu. Rev. Immunol. 22, 657-82; Fredholm, 2001, Pharmacol. Rev. 53, 527-552). As a result, the tumor has local areas of hypoxia and adenosine accumulates to high levels (Hockel, 2001, J. Natl. Cancer Inst. 93, 266-76). In particular, it is recognized that significant levels of adenosine are present in the extracellular fluid of solid tumors (Blay, 1997, Cancer Res., 57, 2602-5), suggesting a role for this nucleoside in tumor growth.

Adenosine has been linked to tumor development. Increased adenosine concentration has been reported inside tumoral masses. It has been speculated that it represents the anti-tumor agent that prevents tumor growth in muscle tissue in vivo and that impairs malignant cell growth and survival in vitro. However, it is known that adenosine acts as cyto-protective agent during ischemic damage in brain and heart. Adenosine is known to be released in hypoxia. Numerous studies have shown adenosine to protect cells in the heart from ischemic damage.

Adenosine has been shown to have protective roles in numerous animal models and in man (Am. J. Cardiol. 79(12A):44-48 (1997). For example, in the heart, both the A₁ and A₃ receptors offer protection against ischemia (Am. J. Physiol., 273(42)H501-505 (1997)). However, it is the A₃ receptor that offers sustained protection against ischemia (PNAS 95:6995-6999 (1998)). The ability of adenosine to protect tumor cells against hypoxia has not been recognized by others prior to the instant invention.

Adenosine interacts with cell surface receptors that are glycoproteins coupled to different members of G protein family. By now four adenosine receptors have been cloned and characterised: A₁, A_{2A}, A_{2B} and A₃. Selective antagonists for the A₃ receptor have been proposed for use as anti-inflammatory and antiischemic agents in the brain. Recently, A₃ antagonists have been under development as antiasthmatic, antidepressant, anti-arrhythmic, renal protective, antiparkinson and cognitive enhancing drugs. For example, U.S. Pat. No. 5,646,156 to Marlene Jacobson et al. describes the inhibition of eosinophil activation by using selected A₃ antagonists.

Recent studies in myocytes have shown the adenosine A₃ receptors to be responsible for long-term protection against ischemia (Liang and Jacobson, PNAS, 1998, 95:6995-6999). While the present inventors have hypothesized that adenosine plays a protective role in other cell types, including tumor cells, in addition to myocytes, no efforts have been made to limit the protective effect of adenosine on tumor cells.

3.2 HIF-1 Biology

Hypoxia-inducible factor (HIF)-1 is a transcription factor that functions as a master regulator of oxygen homeostasis (Semenza, 2001, Trends Mol. Med. 7, 345-350.

HIF-1 is a heterodimer composed of an inducibly-expressed HIF-1α subunit and a constitutively-expressed HIF-1β subunit (Epstein, 2001, Cell, 107, 43-54). HIF-1α and HIF-1β mRNAs are constantly expressed under normoxic and hypoxic conditions (Wiener, 1996 Biochem. Biophys. Res. Commun. 225, 485-488). The unique feature of HIF-1 is the regulation of HIF-1α expression: it increases as the cellular O₂ concentration is decreased (Cramer, 2003, Cell, 112, 645-657, Pugh, 2003, Nat. Med. 9, 677-84). During normoxia, HIF-1α is rapidly degraded by the ubiquitin proteasome system, whereas exposure to hypoxic conditions prevents its degradation (Minchenko, 2002 J. Biol. Chem., 277, 6183-6187; Semenza, 2000, J. Appl. Physiol., 88, 1474-1480).

A growing body of evidence indicates that HIF-1 contributes to tumor progression and metastasis (Hopfl, 2004, Am. J. Physiol. Regul. Integr. Comp. Physiol. 286, R608-23; Welsh, 2003, Curr. Cancer Drug Targets. 3, 391-405). Immunohistochemical analyses have shown that HIF-1α is present in higher levels in human tumors than in normal tissues (Zhong, 2000, Cancer Res. 60, 1541-5). Tumor progression is associated with adaptation to hypoxia, and there is an inverse correlation between tumor oxygenation and clinical outcome (Pugh, 2003, Ann. Med. 35, 380-90.; Semenza, 2000 J. Appl. Physiol., 88, 1474-1480). In particular, the levels of HIF-1 activity in cells are correlated with tumorigenicity and angiogenesis in nude mice (Chen, 2003, Am. J. Pathol. 162,1283-91). Tumor cells lacking HIF-1 expression are markedly impaired in their growth and vascularization (Carmeliet, 1998, Nature 394, 485-90; Jiang, 1997, Cancer Res., 57, 5328-5335; Maxwell, 1997, Proc. Natl. Acad. Sci. U.S.A., 94, 8104-8109; Ryan, 1998 EMBO J. 17, 3005-3015). Therefore, since HIF-1α expression and activity appear central to tumor growth and progression, HIF-1 inhibition has become an appropriate anticancer target (Kung, 2000, Nat. Med. 6, 1335-40).

3.3 Diseases of Relevance

3.3.1 Cancer

A neoplasm, or tumor, is a neoplastic mass resulting from abnormal uncontrolled cell growth which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

More than 1.2 million Americans develop cancer each year. Cancer is the second leading case of death in the United States and if current trends continue, cancer is expected to be the leading cause of death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime.

A cure for cancer has yet to be found. Current treatment options, such as surgery, chemotherapy and radiation treatment, are oftentimes either ineffective or present serious side effects.

Currently, cancer therapy may involve surgery, chemotherapy, hormonal therapy and/or radiation treatment to eradicate neoplastic cells in a patient (See, for example, Stockdale, 1998, “Principles of Cancer Patient Management”, in Scientific American: Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although can be effective, is often used to prevent or delay recurrence of cancer after other treatments have removed the majority of the cancer cells. Biological therapies/immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (See, for example, Gilman et al., Goodman and Gilman's: The Pharmacological Basis of Therapeutics, Eighth Ed. (Pergamom Press, New York, 2001, 10^{th} ed.)). These agents, which include alkylating agents, such as nitrosourea, anti-metabolites, such as methotrexate and hydroxyurea, and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (See, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for treatment of cancer that has proved refractory to standard cancer treatments, such as surgery, radiation therapy, chemotherapy, and hormonal therapy. A promising alternative is immunotherapy, in which cancer cells are specifically targeted by cancer antigen-specific antibodies. Major efforts have been directed at harnessing the specificity of the immune response, for example, hybridoma technology has enabled the development of tumor selective monoclonal antibodies (See Green M. C. et al., 2000 Cancer Treat Rev., 26: 269-286; Weiner L M, 1999 Semin Oncol. 26(suppl. 14):43-51), and in the past few years, the Food and Drug Administration has approved the first MAbs for cancer therapy: However, there is still an unmet need for the treatment of cancers.

In the current chemotherapeutic treatment of human cancer, side effects associated with the chemotherapeutic agent are often severe. The use of paclitaxel or docetaxel (both taxane family medicaments) for the treatment of neoplastic diseases is limited by acute hypersensitivity reactions experienced in many patients. For example, docetaxel administered at 100 mg/m² causes acute hypersensitivity reaction in 13% of patients, and severe hypersensitivity reaction in 1.2%. Due to these reactions, patients are normally premedicated with oral corticosteroids.

Similarly, side effects associated with the use of vinca alkaloids often limit the useful dosages. For example, vincristine has been reported to be dose limited due to neurotoxicity. An enhancing agent providing a neuro-protective effect is therefore desirable. Chemotherapeutic agents are also costly to produce and provide to patients. If the agents can be used at reduced dosages, both the cost and the extent of undesirable side effects can be similarly reduced. Another often-encountered challenge with chemotherapeutic treatment is limitations in effectiveness due to the cancerous growth or cells developing multidrug-resistance (MDR). As most cancer cells are genetically unstable they are prone to mutations likely to produce drug resistant cells.

Multi-drug resistance is the name given to the circumstance when a disease does not respond to a treatment drug or drugs. MDR can be either intrinsic, which means the disease has never been responsive to the drug or drugs, or it can be acquired, which means the disease ceases responding to a drug or drugs that the disease had previously been responsive to MDR is characterized by cross-resistance of a disease to more than one functionally and/or structurally unrelated drugs. MDR in the field of cancer, is discussed in greater detail in “Detoxification Mechanisms and Tumor Cell Resistance to Anticancer Drugs,” by Kuzmich and Tew, particularly section VII “The Multidrug-Resistant Phenotype (MDR),” Medical Research Reviews, Vol. 11, No. 2, 185-217, (Section VII is at pp. 208-213) (1991); and in “Multidrug Resistance and Chemosensitization: Therapeutic Implications for Cancer Chemotherapy,” by Georges, Sharom and Ling, Advances in Pharmacology, Vol. 21, 185-220 (1990).

Different MDR mechanisms have been reported. One form of multi-drug resistance (MDR) is mediated by a membrane bound 170-180 kD energy-dependent efflux pump designated as pleitotropic-glycoprotein or P-glycoprotein (P-gp) that is codified by MDR-1 gene (Endicott J A, Annu Rev Biochem 1989). P-glycoprotein has been shown to play a major role in the intrinsic and acquired resistance of a number of human tumors. Drugs that act as substrates for and are consequently detoxified by P-gp include the vinca alkaloids (vincristine and vinblastine), anthracyclines (Adriamycin), and epipodophyllotoxins (etoposide).

Recently, MDR-1 gene has been identified as an additional risk factor in advanced ovarian cancer. In the study by D. S. Alberts et al., patients with phase III ovarian cancer were screened for MDR-1. Ovarian cancer patients with high levels of MDR-1 survived an average of 9.8 months. The patients having low or no MDR-1 expression survived an average of 30 months or more.

While P-gp associated MDR is a major factor in tumor cell resistance to chemotherapeutic agents, it is clear that the phenomenon of MDR is multifactorial and involves a number of different mechanisms. One such alternative pathway for resistance to anthracyclines involves the emergence of a 190 kD protein (p190) that is not P-gp. See, T. McGrath, et al., Biochemical Pharmacolology, 38:3611 (1989). The protein p190 is not found exclusively on the plasma membrane but rather appears to be localized predominantly in the endoplasmic reticulum. See, e.g., Marquardt, 1992 Cancer Research, 52:3157.

The work of Cole and Deeley isolated a single open reading frame encoding a protein of 1531 amino acids designated as multidrug resistance-associated protein (MRP). As reported by Fan et al., the MRP protein is thought to be the same as the 190 kD protein. MRP has been observed in breast cancer, human leukemia, small cell lung cancer, human large cell lung cancer, fibrosarcoma, adenocarcinoma, thyroid cancer, and cervical cancer. Chen reports that MRP is associated with multi-drug resistance to camptothecin and its analogs.

Other MDR has been reported that is neither P-gp nor MRP related. (Kellen editor, Alternative Mechanisms of Multidrug Resistance in Cancer, Birkhauser, 1995). The results obtained by inventors are consistent for using adenosine A₃ antagonists for countering P-gp or MRP multi-drug resistance, but not other forms of MDR.

α-[3-[[2-(3,4-Dimethoxyphenyl)ethyl]methylamino]propyl]-3,4-dimethoxy-α-(1-methylethyl)benzeneacetonitrile (Verapamil) has been utilized as a medicament to counter the effect of P-gp associated MDR. Verapamil blocks L-type calcium channels and is used as a potent vasodilator of coronary and peripheral vessels and decreases myocardial oxygen consumption. Due to the Verapamil physiological effects, MDR use of Verapamil must be limited to patients not having low blood pressure, congestive heart failure, sinoatrial (SA) or atrioventricular (AV) node conduction disturbances, digitalis toxicity, Wolff-Parkinson-White syndrome, and further not being medicated with beta-blockers or Quinidine.

Recognizing that P-gp is also adenosine-5′-triphosphate (ATP) dependent, another proposed method of countering MDR is to inhibit ATP synthesis in the cancerous cells. U.S. Pat. No. 6,210,917 to Carson et al. discloses the use of L-alanosine and other adenosine kinase inhibitors for countering MDR. In addition U.S. Pat. No. 6,391,884 identifies ATP-depleting agents including 2-deoxyglucose, cyanine, oligomycin, valinomycin and azide, as well as salts and derivatives thereof. Approaches relying upon ATP depletion or inhibition in countering MDR have yet to receive clinical success.

As a result, it is seen that there is a need for chemotherapeutic agent enhancers and compounds that prevent or counter MDR either alone or with ATP depleting agents in cancer treatment but without the limitations imposed by Verapamil.

The use of a combination of therapeutic agents is common in the treatment of neoplastic diseases. For example, paclitaxel (Taxol™) has been approved by the U.S. FDA for use with cisplatin in the treatment of ovarian carcinoma. U.S. Pat. No. 5,908,835 to Bissery et al. claims synergy of using paclitaxel or docetaxel in combination with an anthracycline antibiotic such as daunorubicin or doxorubicin. Similarly, U.S. Pat. No. 5,728,687 to Bissery et al. claims synergy of using paclitaxel or docetaxel in combination with an alkylating agent, epidophyllotoxin, antimetabolite, or vinca alkaloid. However, such combinations heretofore have not included the use of adenosine receptor antagonists and in particular A₃ receptor antagonists.

It is therefore an object of the present invention to provide compositions and methods of enhancing the treatment of neoplastic cells by minimizing or eliminating the protective effect of adenosine on cells with the use of adenosine A₃ receptor antagonists. It is a further object of the present invention to provide compositions and methods suitable for countering P-gp and/or MRP associated multi-drug resistance. It is further an object of the present invention to provide compositions and methods that reduce side effects of other currently used anti-cancer agents and/or therapies, in particular taxane induced hypersensitivity.

### 4. SUMMARY OF THE INVENTION

The present invention is based, in part, on the surprising discovery by the inventors that adenosine, a purine nucleoside present within hypoxic regions of solid tumors, modulates hypoxia-inducible factor 1 (HIF-1) expression. HIF-1, a heterodimeric transcription factor composed of HIF-1α and HIF-1β subunits, is involved in tumor growth and angiogenesis (for reviews see, e.g., Semenza 2000, Nature, 3: 721-32). The inventors have found that in the human A375 melanoma cell line adenosine up-regulates HIF-1α protein expression in response to hypoxia in a dose- and time-dependent manner. The response to adenosine was not blocked by A₁, A_{2A} or A_{2B} receptor antagonists, while it was abolished by A₃ receptor antagonists. The inventors have found that Cl-IB-MECA, an adenosine analogue binding with high affinity to A₃ receptors, mimicked adenosine effect in hypoxic cells. Furthermore, A₃ receptor antagonists prevented HIF-1α protein accumulation in response to A₃ receptor stimulation. Although not intending to be bound by a particular mechanism of action, the response to adenosine is generated at the cell surface since the inhibition of A₃ receptor expression, by using small interfering RNA, abolished the nucleoside effects. The main intracellular signaling pathways sustained by A₃ receptor stimulation in hypoxia involve p44/p42 mitogen-activated protein kinase (MAPK) and p38 MAPK activation. The inventors have thus found for the first time that adenosine plays a critical role in HIF-1α regulation in hypoxia.

The inventors have found that compounds which are antagonists of the adenosine receptors, preferably the A₃ receptor, inhibit the protective effect of adenosine on growing tumor cells when such cells are starved of oxygen (i.e., before an adequate vasculature is developed, or when anti-angiogenesis agents are administered. Although not intending to be bound by a particular mechanism of action, the antagonists of the invention can bind in the same site as adenosine, or can be allosteric antagonists (i.e., bind at a site different from where adenosine binds, but adversely affect the ability of adenosine to bind to the site or adversely affect the ability of adenosine, once bound to the adenosine receptors, particularly A₃ receptors, to protect growing tumor cells).

Accordingly, the present invention relates to methods for the treatment, prevention, and/or management of diseases or disorders associated with overexpression of HIF-1α and/or increased HIF-1α activity (e.g., cancer, respiratory disorders such as asthma and obstructive pulmonary disorders) by using adenosine receptor antagonists, particularly A₃ receptor antagonists alone or in combination with A₁, A_{2A}, A_{2B} receptor antagonists. Although the A₁, A_{2A} and A_{2B} receptor antagonists may act through different pathways than the A₃ receptor antagonist, the combination of these antagonists with A₃ receptor antagonist may be beneficial in the treatment, prevention, and/or management of diseases or disorders, e.g., cancer.

In most preferred embodiments, the methods of the invention relate to treatment, prevention, and/or management of diseases or disorders associated with overexpression of HIF-1α and/or increased HIF-1α activity by using adenosine receptor antagonists, particularly A₃ receptor antagonists alone. Without being bound to a particular mechanism of action, administration of antagonists for the adenosine receptors, particularly A₃ receptor antagonists, antagonizes the protective effects against hypoxia and renders those cells susceptible to destruction due to hypoxia. Since the adenosine receptors, in particular, the A₃ receptor, are responsible for sustained cellular protection against ischemia, antagonists for the adenosine receptors, particularly A₃ receptors are particularly effective in enhancing the activity of anti-tumor agents.

The methods and compositions of the invention comprising A₃ receptor antagonists are particularly useful when the levels of HIF-1α expression and/or activity are elevated above the standard or background level, as determined using methods known to those skilled in the art and disclosed herein. As used herein, “elevation” of a measured level of HIF-1α relative to a standard level means that the amount or concentration of HIF-1α in a sample or subject is greater in a subject or sample relative to the standard as detected by any method now known in the art or to be developed in the future for measuring HIF-1α levels. For example, elevation of the measured level relative to a standard level may be any statistically significant detectable elevation. Such an elevation in HIF-1α expression and/or activity may include, but is not limited to about a 10%, about a 20%, about a 40%, about an 80%, about a 2-fold, about a 4-fold, about an 10-fold, about a 20-fold, about a 50-fold, about a 100-fold, about a 2 to 20 fold, 2 to 50 fold, 2 to 100 fold, 20 to 50 fold, 20 to 100 fold, elevation, relative to the standard. The term “about” as used herein, refers to levels of elevation of the standard numerical value plus or minus 10% of the numerical value.

The therapeutic methods of the invention comprising administering a therapeutically effective amount of an A₃ receptor antagonist of the invention improve the therapeutic efficacy of treatment for diseases or disorders associated with overexpression of HIF-1α and/or increased HIF-1α activity (e.g., cancer, respiratory disorders such as asthma and obstructive pulmonary disorders) relative to the traditional modes of such therapies. Preferably the methods of the invention reduce the HIF-1α level to the background level within one day, one week, 1 month, or 2 months, of the commencement of the therapeutic regime. In a most preferred embodiment, the methods of the invention result in a reduction of HIF-1α level to the background level. The invention encompasses reduction of the HIF-1α level to a level which is within about 10%, about 20%, about 30%, about 40%, about 50% of the background level; about a 2-fold, about a 4-fold, about an 10-fold, about a 20-fold, about a 50-fold, about a 100-fold, about a 2 to 20 fold, 2 to 50 fold, 2 to 100 fold, 20 to 50 fold, 20 to 100 fold of the background level. Preferably, once the methods of the invention reduce the level of HIF-1α to a particular level, that level is maintained during the treatment regimen, such that the maintained level of HIF-1α is sufficient and effective to result in regression of the disease, e.g., cancer.

In a preferred specific embodiment, the invention encompasses a method for treatment, prevention and/or management of diseases or disorders associated with overexpression of HIF-1α and/or increased HIF-1α activity (e.g., cancer, respiratory disorders such as asthma and obstructive pulmonary disorders) comprising administering a therapeutically and/or prophylactically effective amount of an A₃ receptor antagonist compound as disclosed herein alone or in combinationo with other therapeutic or prophylatic agents.

The invention also encompasses a method for determining the prognosis of a a disease or disorder associated with overexpression of HIF-1α and/or increased HIF-1α activity in a subject. Preferably, the subject is human. In yet another embodiment, the subject has been previously treated with a therapy regimen. The invention encompasses measuring a level of HIF-1α in a subject to determine if the subject is in need of the therapeutic and or prophylactic methods of the inventon. The invention encompasses measuring a level of HIF-1α in a sample obtained from the subject and comparing the level measured to a standard level, wherein elevation of the measured level of at least one HIF-1α relative to the standard level indicates that the subject is at an increased risk for progression of the disease or disorder, e.g., metastasis of the cancer. The HIF-1α level may be measured using at least one of the methods for measuring and detecting HIF-1α such as those known to those skilled in the art and exemplified herein.

The invention encompasses compounds which are A₃ or A₁ receptor antagonists for use in the methods of the invention. Examples of such compounds are disclosed in U.S. Pat. Nos. 6,326,390; 6,407,236; 6,448,253; 6,358,964; and U.S. Publication Nos. 2003/0144266 and 2004/0067932; all of which are incorporated herein by reference in their entireties. Furthermore, additional examples of such compounds are disclosed in the list of references in Table 1, the disclosures of which are hereby incorporated by reference in their entireties.

TABLE 1
A₃ Receptor Antagonists

The A₃ adenosine receptor agonist 2-Cl-IB-MECA facilitates epileptiform discharges in
the CA3 area of immature rat hippocampal slices. Laudadio, Mark Anthony;
Psarropoulou, Caterina. Department of Paediatrics, Ste-Justine Hospital Research
Centre, Universite de Montreal, Montreal, QC, Can. Epilepsy Research (2004), 59(2-3),
83-94.
Structure-activity relationships of adenosine A₃ receptor ligands: new potential therapy
for the treatment of glaucoma. Okamura, Takashi; Kurogi, Yasuhisa; Hashimoto, Kinji;
Sato, Seiji; Nishikawa, Hiroshi; Kiryu, Kimio; Nagao, Yoshimitsu. Pharmaceutical
Technology Institute, Otsuka Pharmaceutical Factory, Inc., Naruto, Tokushima, Japan.
Bioorganic & Medicinal Chemistry Letters (2004), 14(14), 3775-3779.
QSAR of adenosine receptor antagonists. Part 3: exploring physicochemical requirements
for selective binding of 1,2,4-triazolo[5,1-i]purine derivatives with human adenosine A₃
receptor subtype. Roy, Kunal; Leonard, J. Thomas; Sengupta, Chandana. Drug
Theoretics and Cheminformatics Laboratory, Division of Medicinal and Pharmaceutical
Chemistry, Department of Pharmaceutical Technology, Jadavpur University, Kolkata,
India. Bioorganic & Medicinal Chemistry Letters (2004), 14(14), 3705-3709.
WO 2004051264 A1
1,2,4-Triazolo[4,3-a]quinoxalin-1-one Moiety as an Attractive Scaffold To Develop New
Potent and Selective Human A3 Adenosine Receptor Antagonists: Synthesis,
Pharmacological, and Ligand-Receptor Modeling Studies. Colotta, Vittoria; Catarzi,
Daniela; Varano, Flavia; Calabri, Francesca Romana; Lenzi, Ombretta; Filacchioni,
Guido; Martini, Claudia; Trincavelli, Letizia; Deflorian, Francesca; Moro, Stefano.
Dipartimento di Scienze Farmaceutiche, Universita degli Studi di Firenze, Sesto
Fiorentino, Italy. Journal of Medicinal Chemistry (2004), 47(14), 3580-3590.
WO 2004046146 A1
Facile synthesis of fused 1,2,4-triazolo[1,5-c]pyrimidine derivatives as human adenosine
A3 receptor ligands. Okamura, Takashi; Kurogi, Yasuhisa; Hashimoto, Kinji;
Nishikawa, Hiroshi; Nagao, Yoshimitsu. Pharmaceutical Technology Institute, Otsuka
Pharmaceutical Factory, Inc., Naruto, Tokushima, Japan. Bioorganic & Medicinal
Chemistry Letters (2004), 14(10), 2443-2446.
New highly potent and selective adenosine A3 receptor antagonists. Press, Neil J.;
Keller, Thomas H.; Tranter, Pamela; Beer, David; Jones, Ken; Faessler, Alexander;
Heng, Richard; Lewis, Christine; Howe, Trevor; Gedeck, Peter; Mazzoni, Lazzaro;
Fozard, John R. Respiratory Disease Area, Novartis Horsham Research Center, West
Sussex, UK. Current Topics in Medicinal Chemistry (Sharjah, United Arab Emirates)
(2004), 4(8), 863-870.
Role of direct RhoA-phospholipase D interaction in mediating adenosine-induced
protection from cardiac ischemia. Mozzicato, Susan; Joshi, Bhalchandra V.; Jacobson,
Kenneth A.; Liang, Bruce T. Dep. of Cardiol., Univ. of Connecticut Health Cent.,
Farmington, CT, USA. FASEB Journal (2004), 18(2), 406-408.
Synthesis and structure-activity relationships of 4-cycloalkylamino-1,2,4-triazolo[4,3-
a]quinoxalin-1-one derivatives as A₁ and A₃ adenosine receptor antagonists. Colotta,
Vittoria; Catarzi, Daniela; Varano, Flavia; Filacchioni, Guido; Martini, Claudia;
Trincavelli, Letizia; Lucacchini, Antonio. Dipartimento di Scienze Farmaceutiche,
Universita' di Firenze, Florence, Italy. Archiv der Pharmazie (Weinheim, Germany)
(2004), 337(1), 35-41.
Structural determinants of efficacy at A₃ adenosine receptors: modification of the ribose
moiety. Gao, Zhan-Guo; Jeong, Lak Shin; Moon, Hyung Ryong; Kim, Hea Ok; Choi,
Won Jun; Shin, Dae Hong; Elhalem, Eleonora; Comin, Maria J.; Melman, Neli;
Mamedova, Liaman; Gross, Ariel S.; Rodriguez, Juan B.; Jacobson, Kenneth A. National
Institute of Diabetes and Digestive and Kidney Diseases, Molecular Recognition Section,
Laboratory of Bioorganic Chemistry, National Institutes of Health, Bethesda, MD, USA.
Biochemical Pharmacology (2004), 67(5).
U.S. Pat. No. 6,686,366
Structure-activity relationships of thiazole and thiadiazole derivatives as potent and
selective human adenosine A3 receptor antagonists. Jung, Kwan-Young; Kim, Soo-
Kyung; Gao, Zhan-Guo; Gross, Ariel S.; Melman, Neli; Jacobson, Kenneth A.; Kim,
Yong-Chul. Department of Life Science, Laboratory of Drug Discovery, Kwangju
Institute of Science and Technology, Gwangju, S. Korea. Bioorganic & Medicinal
Chemistry (2004), 12(3), 613-623.
Extracellular ATP and adenosine induce cell apoptosis of human hepatoma Li-7A cells
via the A3 adenosine receptor. Wen, Long T.; Knowles, Aileen F. Department of
Biology, San Diego State University, San Diego, CA, USA. British Journal of
Pharmacology (2003), 140(6), 1009-1018.
Alteration of A₃ adenosine receptors in human neutrophils and low frequency
electromagnetic fields. Varani, Katia; Gessi, Stefania; Merighi, Stefania; Lannotta,
Valeria; Cattabriga, Elena; Pancaldi, Cecilia; Cadossi, Ruggero; Borea, Pier Andrea.
Pharmacology Unit, Department of Clinical and Experimental Medicine, University of
Ferrara, Ferrara, Italy. Biochemical Pharmacology (2003), 66(10), 1897-1906.
A₃ adenosine receptors. Jacobson, Kenneth A.; Tchilibon, Susanna; Joshi, Bhalchandra V.;
Gao, Zhan-Guo. Molecular Recognition Section, Laboratory of Bioorganic
Chemistry, National Institute of Diabetes, Digestive and Kidney Diseases, Bethesda, MD,
USA. Annual Reports in Medicinal Chemistry (2003), 38 121-130.
Brief, repeated, oxygen-glucose deprivation episodes protect neurotransmission from a
longer ischemic episode in the in vitro hippocampus: Role of adenosine receptors.
Pugliese, Anna Maria; Latini, Serena; Corradetti, Renato; Pedata, Felicita. Department of
Preclinical and Clinical Pharmacology, University of Florence, Florence, Italy. British
Journal of Pharmacology (2003), 140(2), 305-314.
Protection from myocardial stunning by ischaemia and hypoxia with the adenosine A₃
receptor agonist, IB-MECA. Maddock, Helen L.; Gardner, Neil M.; Khandoudi,
Nassirah; Bril, Antoine; Broadley, Kenneth J. Welsh School of Pharmacy, Department of
Pharmacology, Cardiff University, Cardiff, Cathays Park, Wales, UK. European Journal
of Pharmacology (2003), 477(3), 235-245.
Hypersensitivity of pulmonary C fibers induced by adenosine in anesthetized rats. Gu,
Qihai; Ruan, Ting; Hong, Ju-Lun; Burki, Nausherwan; Lee, Lu-Yuan. Department of
Physiology, University of Kentucky Medical Center, Lexington, KY, USA. Journal of
Applied Physiology (2003), 95(3), 1315-1324.
New strategies for the synthesis of A₃ adenosine receptor antagonists. Baraldi, Pier
Giovanni; Bovero, Andrea; Fruttarolo, Francesca; Romagnoli, Romeo; Tabrizi, Mojgan
Aghazadeh; Preti, Delia; Varani, Katia; Borea, Pier Andrea; Moorman, Allan R.
Dipartimento di Scienze Farmaceutiche, Universita di Ferrara, Ferrara, Italy. Bioorganic
& Medicinal Chemistry (2003), 11(19), 4161-4169.
Pyrazolo[4,3-e]-1,2,4-triazolo[1,5-c]pyrimidine Derivatives as Adenosine Receptor
Antagonists. Influence of the N5 Substituent on the Affinity at the Human A₃ and A_{2B}
Adenosine Receptor Subtypes: A Molecular Modeling Investigation. Pastorin, Giorgia;
Da Ros, Tatiana; Spalluto, Giampiero; Deflorian, Francesca; Moro, Stefano; Cacciari,
Barbara; Baraldi, Pier Giovanni; Gessi, Stefania; Varani, Katia; Borea, Pier Andrea.
Dipartimento di Scienze Farmaceutiche, Universita degli Studi di Trieste, Trieste, Italy.
Journal of Medicinal Chemistry (2003), 46(20), 4287-4296.
QSAR of adenosine receptor antagonists II: Exploring physicochemical requirements for
selective binding of 2-arylpyrazolo[3,4-c]quinoline derivatives with adenosine A₁ and A₃
receptor subtypes. Roy, Kunal. Drug Theoretics and Cheminformatics Lab, Division of
Medicinal and Pharmaceutical Chemistry, Department of Pharmaceutical Technology,
Jadavpur University, Calcutta, India. QSAR & Combinatorial Science (2003), 22(6),
614-621.
Functional expression of adenosine A_{2A} and A₃ receptors in the mouse dendritic cell line
XS-106. Dickenson, John M.; Reeder, Steve; Rees, Bob; Alexander, Steve; Kendall,
Dave. School of Science, Faculty of Science and Mathematics, Department of Life
Sciences, Nottingham Trent University, Nottingham, UK. European Journal of
Pharmacology (2003), 474(1), 43-51.
Synthesis and structure-activity relationships of a new set of 1,2,4-triazolo[4,3-
a]quinoxalin-1-one derivatives as adenosine receptor antagonists. Colotta, Vittoria;
Catarzi, Daniela; Varano, Flavia; Filacchioni, Guido; Martini, Claudia; Trincavelli,
Letizia; Lucacchini, Antonio. Dipartimento di Scienze Farmaceutiche, Universita' di
Firenze, Florence, Italy. Bioorganic & Medicinal Chemistry (2003), 11(16), 3541-3550.
A₃ adenosine and CB1 receptors activate a PKC-sensitive Cl-current in human
nonpigmented ciliary epithelial cells via a G-coupled MAPK signaling pathway. Shi,
Chanjuan; Szczesniak, Anna; Mao, Lucy; Jollimore, Christine; Coca-Prados, Miguel;
Hung, Orlando; Kelly, Melanie E. M. Laboratory for Retina and Optic Nerve Research,
Dalhousie University, Halifax, NS, Can. British Journal of Pharmacology (2003),
139(3), 475-486.
WO 2003053969 A1
WO 2003053968 A1
Characterization of adenosine receptor(s) involved in adenosine-induced
bronchoconstriction in an allergic mouse model. Fan, Ming; Qin, Weixi; Mustafa, S. Jamal.
Department of Pharmacology and Toxicology, Brody School of Medicine, East
Carolina University, Greenville, NC, USA. American Journal of Physiology (2003),
284(6, Pt. 1), L1012-L1019.
QSAR of adenosine receptor antagonists, I: Exploration of receptor interaction sites of
1,2-dihydro-2-phenyl-1,2,4-triazolo[4,3-a]quinoxalin-1-one derivatives using AM1
calculations. Roy, Kunal. Drug Theoretics and Cheminformatics Lab, Division of
Medicinal and Pharmaceutical Chemistry, Department of Pharmaceutical Technology,
Jadavpur University, Kolkata, India. Indian Journal of Chemistry, Section B: Organic
Chemistry Including Medicinal Chemistry (2003), 42B(6), 1485-1496.
Activation of murine lung mast cells by the adenosine A₃ receptor. Zhong, Hongyan;
Shlykov, Sergiy G.; Molina, Jose G.; Sanborn, Barbara M.; Jacobson, Marlene A.; Tilley,
Stephen L.; Blackburn, Michael R. Department of Biochemistry and Molecular Biology,
University of Texas-Houston Medical School, Houston, TX, USA. Journal of
Immunology (2003), 171(1), 338-345.
Modeling the response of the asthmatic airways to adenosine: Mechanisms and receptors.
Fozard, John R.; Tigani, Bruno; Wolber, Cedric; Williams, Iwan; Mazzoni, Lazzaro;
Hannon, Jason P. Research Department, Novartis Pharma AG., Basel, Switz. Drug
Development Research (2003), 59(1), 23-29.
9-Ethyladenine derivatives as adenosine receptor antagonists: 2- and 8-substitution
results in distinct selectivities. Klotz, Karl-Norbert; Kachler, Sonja; Lambertucci, Catia;
Vittori, Sauro; Volpini, Rosaria; Cristalli, Gloria. Institut fuer Pharmakologie und
Toxikologie, Universitaet Wuerzburg, Wuerzburg, Germany. Naunyn-Schmiedeberg's
Archives of Pharmacology (2003), 367(6), 629-634.
Recent developments in the field of A₃ adenosine receptor antagonists. Baraldi, Pier
Giovanni; Tabrizi, Mojgan Aghazadeh; Fruttarolo, Francesca; Bovero, Andrea; Avitabile,
Barbara; Preti, Delia; Romagnoli, Romeo; Merighi, Stefania; Gessi, Stefania; Varani,
Katia; Borea, Pier Andrea. Dipartimento di Scienze Farmaceutiche, Universita di
Ferrara, Ferrara, Italy. Drug Development Research (2003), 58(4), 315-329.
Alteration of the purinergic modulation of enteric neurotransmission in the mouse ileum
during chronic intestinal inflammation. De Man, Joris G.; Seerden, Tom C.; De Winter,
Benedicte Y.; Van Marck, Eric A.; Herman, Arnold G.; Pelckmans, Paul A. Division of
Gastroenterology, Faculty of Medicine, University of Antwerp (UIA), Antwerp, Belg.
British Journal of Pharmacology (2003), 139(1), 172-184.
WO 2003048120 A2
Improved, efficient synthesis for multigram-scale production of PSB-10, a potent
antagonist at human A3 adenosine receptors. Burbiel, Joachim; Thorand, Mark; Muller,
Christa E. Pharmazeutisches Institut, Rheinische Friedrich-Wilhelms-Universitat Bonn,
Bonn, Germany. Heterocycles (2003), 60(6), 1425-1432.
U.S. Pat. No. 6,586,413
U.S. Pat. No. 6,211,165
Recent developments in the field of A_{2A} and A₃ adenosine receptor antagonists. Baraldi,
Pier Giovanni; Tabrizi, Mojgan Aghazadeh; Bovero, Andrea; Avitabile, Barbara; Preti,
Delia; Fruttarolo, Francesca; Romagnoli, Romeo; Varani, Katia; Borea, Pier Andrea.
Dipartimento di Scienze Farmaceutiche, Universita di Ferrara, Ferrara, Italy. European
Journal of Medicinal Chemistry (2003), 38(4), 367-382.
U.S. Pat. No. 6,673,802
*Allosteric modulation of A₃ adenosine receptors. Jacobson, Kenneth A.; Kim, Soo-
Kyung; Ijzerman, Adriaan P.; Gao, Zhan-Guo. Molecular Recognition Section, NIDDK,
NIH, Bethesda, MD, USA. Abstracts of Papers, 225th ACS National Meeting, New
Orleans, LA, United States, March 23-27, 2003 (2003), MEDI-316. Publisher:
American Chemical Society, Washington, D.C.
Design, Synthesis, and Biological Evaluation of C9- and C2-Substituted Pyrazolo[4,3-e]-
1,2,4-triazolo[1,5-c]pyrimidines as New A2A and A3 Adenosine Receptor Antagonists.
Baraldi, Pier Giovanni; Fruttarolo, Francesca; Tabrizi, Mojgan Aghazadeh; Preti, Delia;
Romagnoli, Romeo; El-Kashef, Hussein; Moorman, Allan; Varani, Katia; Gessi,
Stefania; Merighi, Stefania; Borea, Pier Andrea. Dipartimento di Scienze Farmaceutiche
and Dipartimento di Medicina Clinica e Sperimentale-Sezione di Farmacologia,
Universita di Ferrara, Ferrara, Italy. Journal of Medicinal Chemistry (2003), 46(7),
1229-1241.
Differential allosteric modulation by amiloride analogues of agonist and antagonist
binding at A1 and A3 adenosine receptors. Gao, Zhan-Guo; Melman, Neli; Erdmann,
Andreas; Kim, Seong Gon; Muller, Christa E.; IJzerman, Adriaan P.; Jacobson, Kenneth A.
NIDDK, Laboratory of Bioorganic Chemistry, Molecular Recognition Section,
National Institutes of Health, Bethesda, MD, USA. Biochemical Pharmacology (2003),
65(4), 525-534.
Adenosine Receptor Subtypes Mediating Coronary Vasodilation in Rat Hearts.
Hinschen, Andrea K.; Rose'Meyer, Roselyn B.; Headrick, John P. Griffith University,
Heart Foundation Research Center, School of Health Science, Gold Coast Campus,
Southport, Australia. Journal of Cardiovascular Pharmacology (2003), 41(1), 73-80.
Contractile responses to adenosine, R-PIA and ovalbumen in passively sensitized guinea-
pig isolated airways. Martin, Timothy J.; Broadley, Kenneth J. Department of
Pharmacology, Welsh School of Pharmacy, Cardiff University, Cardiff, UK. British
Journal of Pharmacology (2002), 137(6), 729-738.
U.S. Pat. No. 2002165197
U.S. Pat. No. 2002115635
WO 2002079204 A1
Structural Determinants of A₃ Adenosine Receptor Activation: Nucleoside Ligands at the
Agonist/Antagonist Boundary. Gao, Zhan-Guo; Kim, Soo-Kyung; Biadatti, Thibaud;
Chen, Wangzhong; Lee, Kyeong; Barak, Dov; Kim, Seong Gon; Johnson, Carl R.;
Jacobson, Kenneth A. Laboratory of Bioorganic Chemistry, Molecular Recognition
Section, National Institutes of Health, National Institute of Diabetes and Digestive and
Kidney Diseases, Bethesda, MD, USA. Journal of Medicinal Chemistry (2002), 45(20),
4471-4484.
New highly potent and selective adenosine A3 receptor antagonists. Press, Neil J.;
Fozard, John R.; Beer, David; Heng, Richard; di Padova, Franco; Tranter, Pamela;
Trifilieff, Alexandre; Walker, Christoph; Keller, Thomas H. Dept. of Chemistry,
Novartis Horsham Research Centre, West Sussex, UK. Abstracts of Papers, 224th ACS
National Meeting, Boston, MA, United States, Aug. 18-22, 2002 (2002), MEDI-419.
Publisher: American Chemical Society, Washington, D.C
U.S. Pat. No. 6,680,322
U.S. Pat. No. 6,664,252
Synthesis, Biological Properties, and Molecular Modeling Investigation of the First
Potent, Selective, and Water-Soluble Human A₃ Adenosine Receptor Antagonist.
Maconi, Anna; Pastorin, Giorgia; Da Ros, Tatiana; Spalluto, Giampiero; Gao, Zhan-guo;
Jacobson, Kenneth A.; Baraldi, Pier Giovanni; Cacciari, Barbara; Varani, Katia; Moro,
Stefano; Borea, Pier Andrea. Dipartimento di Scienze Farmaceutiche, Universita degli
Studi di Trieste, Trieste, Italy. Journal of Medicinal Chemistry (2002), 45(17), 3579-3582.
1,2,4-Triazolo[5,1-i]purine Derivatives as Highly Potent and Selective Human Adenosine
A₃ Receptor Ligands. Okamura, Takashi; Kurogi, Yasuhisa; Nishikawa, Hiroshi;
Hashimoto, Kinji; Fujiwara, Hiroshi; Nagao, Yoshimitsu. Nutrition Research Institute,
Otsuka Pharmaceutical Factory, Inc., Tateiwa, Muya-cho, Naruto, Tokushima, Japan.
Journal of Medicinal Chemistry (2002), 45(17), 3703-3708.
*Selective allosteric enhancement of agonist binding and function at human A₃ adenosine
receptors by a series of imidazoquinoline derivatives. Gao, Zhan-Guo; Kim, Seong Gon;
Soltysiak, Kelly A.; Melman, Neli; Ijzerman, Adriaan P.; Jacobson, Kenneth A.
Molecular Recognition Section, Laboratory of Bioorganic Chemistry, National Institute
of Diabetes and Digestive and Kidney Diseases, National Institutes of Health, Bethesda,
MD, USA. Molecular Pharmacology (2002), 62(1), 81-89.
Imidazo[2,1-i]purin-5-ones and Related Tricyclic Water-Soluble Purine Derivatives:
Potent A_{2A}-and A₃-Adenosine Receptor Antagonists. Mueller, Christa E.; Thorand,
Mark; Qurishi, Ramatullah; Diekmann, Martina; Jacobson, Kenneth A.; Padgett, William L.;
Daly, John W. Pharmaceutical Institute Poppelsdorf, University of Bonn, Bonn,
Germany. Journal of Medicinal Chemistry (2002), 45(16), 3440-3450.
(2-(4-bromophenyl)-7,8-dihydro-4-propyl-1H-imidazo[2,1-i]purin-5(4H)-
onedihydrochloride), a new potent and selective adenosine A3 receptor antagonist. Saki,
Mayumi; Tsumuki, Hiroshi; Nonaka, Hiromi; Shimada, Junichi; Ichimura, Michio.
Kyowa Hakko Kogyo Co., Ltd., Pharmaceutical Research Institute, Sunto-gun, Shizuoka,
Nagaizumi-cho, Japan. European Journal of Pharmacology (2002), 444(3), 133-141.
2-Chloro-N6-cyclopentyladenosine, adenosine A1 receptor agonist, antagonizes the
adenosine A₃ receptor. Gao, Zhan-guo; Jacobson, Kenneth A. Laboratory of Bioorganic
Chemistry, Molecular Recognition Section, National Institutes of Health, National
Institute of Diabetes and Digestive and Kidney Diseases, Bethesda, MD, USA. European
Journal of Pharmacology (2002), 443(1-3), 39-42.
U.S. Pat. No. 6,680,322
U.S. Pat. No. 6,387,913
Adenosine acts through an A₃ receptor to prevent the induction of murine anti-CD3-
activated killer T cells. Hoskin, David W.; Butler, Jared J.; Drapeau, Dennis; Haeryfar,
S. M. Mansour; Blay, Jonathan. Department of Microbiology and Immunology, Faculty
of Medicine, Department of Pathology, Dalhousie University, Halifax, NS, Can.
International Journal of Cancer (2002), 99(3), 386-395.
Binding thermodynamics at the human A₃ adenosine receptor. Merighi, Stefania; Varani,
Katia; Gessi, Stefania; Klotz, Karl-Norbert; Leung, Edward; Baraldi, Pier Giovanni;
Borea, Pier Andrea. Pharmacology Unit, Department of Clinical and Experimental
Medicine, Centro Nazionale di Eccellenza per lo Sviluppo di Metodologie Innovative per
lo Studio ed il Trattamento delle Patologie Infiammatorie, University of Ferrara, Ferrara,
Italy. Biochemical Pharmacology (2002), 63(2), 157-161.
[3H]-8-Ethyl-4-methyl-2-phenyl-(8R)-4,5,7,8-tetrahydro-1H-imidazo[2,1-i]purin-5-one
([3H]PSB-11), a Novel High-Affinity Antagonist Radioligand for Human A3 Adenosine
Receptors. Muller, Christa E.; Diekmann, Martina; Thorand, Mark; Ozola, Vita.
University of Bonn, Pharmaceutical Institute, Bonn, Germany. Bioorganic & Medicinal
Chemistry Letters (2002), 12(3), 501-503.
International union of pharmacology. XXV. Nomenclature and classification of
adenosine receptors. Fredholm, Bertil B.; Ijzerman, Adriaan P.; Jacobson, Kenneth A.;
Klotz, Karl-Norbert; Linden, Joel. Department of Physiology and Pharmacology, Section
of Molecular Neuropharmacology, Karolinska Institutet, Stockholm, Swed.
Pharmacological Reviews (2001), 53(4), 527-552.
Synthesis, Biological Activity, and Molecular Modeling Investigation of New
Pyrazolo[4,3-e]-1,2,4-triazolo[1,5-c]pyrimidine Derivatives as Human A3 Adenosine
Receptor Antagonists. Baraldi, Pier Giovanni; Cacciari, Barbara; Moro, Stefano;
Spalluto, Giampiero; Pastorin, Giorgia; Da Ros, Tatiana; Klotz, Karl-Norbert; Varani,
Katia; Gessi, Stefania; Borea, Pier Andrea. Dipartimento di Scienze Farmaceutiche and
Dipartimento di Medicina Clinica e Sperimentale-Sezione di Farmacologia, Universita
degli Studi di Ferrara, Ferrara, Italy. Journal of Medicinal Chemistry (2002), 45(4), 770-780.
Pharmacological and biochemical characterization of adenosine receptors in the human
malignant melanoma A375 cell line. Merighi, Stefania; Varani, Katia; Gessi, Stefania;
Cattabriga, Elena; Iannotta, Valeria; Ulouglu, Canan; Leung, Edward; Borea, Pier
Andrea. Department of Clinical and Experimental Medicine, Pharmacology Unit, Centro
Nazionale Di Eccellenza Per Lo Sviluppo Di Metodologie Innovative Per Lo Studio Ed II
Trattamento Delle Patologie Infiammatorie, University of Ferrara, Italy. British Journal
of Pharmacology (2001), 134(6), 1215-1226.
7-Substituted 5-Amino-2-(2-furyl)pyrazolo[4,3-e]-1,2,4-triazolo[1,5-c]pyrimidines as
A2A Adenosine Receptor Antagonists: A Study on the Importance of Modifications at
the Side Chain on the Activity and Solubility. Baraldi, Pier Giovanni; Cacciari, Barbara;
Romagnoli, Romeo; Spalluto, Giampiero; Monopoli, Angela; Ongini, Ennio; Varani,
Katia; Borea, Pier Andrea. Dipartimento di Scienze Farmaceutiche, Universita degli
Studi di Ferrara, Ferrara, Italy. Journal of Medicinal Chemistry (2002), 45(1), 115-126.
*A₃ adenosine receptor antagonists. Muller, C. E. Pharmaceutical Institute, University
of Bonn, Bonn, Germany. Mini-Reviews in Medicinal Chemistry (2001), 1(4), 417-427.
CODEN: MMCIAE ISSN: 1389-5575. Journal; General Review written in English.
U.S. Pat. No. 6,303,619
Recent advances in adenosine receptor antagonist research. Hess, Sonja. Laboratory of
Bioorganic Chemistry, National Institute of Diabetes, and Digestive and Kidney
Diseases, National Institutes of Health, Bethesda, MD, USA. Expert Opinion on
Therapeutic Patents (2001), 11(10), 1533-1561.
Pharmacological characterization of adenosine receptors in PGT mouse pineal gland
tumour cells. Suh, Byung-Chang; Kim, Tac-Don; Lee, Jung-Uek; Seong, Je-Kyung;
Kim, Kyong-Tai. Department of Life Science, Division of Molecular and Life Science,
Pohang University of Science and Technology, Pohang, S. Korea. British Journal of
Pharmacology (2001), 134(1), 132-142.
Pharmacological and biochemical characterization of A₃ adenosine receptors in Jurkat T
cells. Gessi, Stefania; Varani, Katia; Merighi, Stefania; Morelli, Anna; Ferrari, Davide;
Leung, Edward; Baraldi, Pier Giovanni; Spalluto, Giampiero; Borea, Pier Andrea.
Department of Clinical and Experimental Medicine, Pharmacology Unit, University of
Ferrara, Ferrara, Italy. British Journal of Pharmacology (2001), 134(1), 116-126.
Introduction of alkynyl chains on C-8 of adenosine led to very selective antagonists of the
A₃ adenosine receptor. Volpini, R.; Costanzi, S.; Lambertucci, C.; Vittori, S.; Klotz, K.-N.;
Lorenzen, A.; Cristalli, G. Dipartimento di Scienze Chimiche, Universita di
Camerino, Camerino, Italy. Bioorganic & Medicinal Chemistry Letters (2001), 11(14),
1931-1934.
Fluorosulfonyl-and bis-(chloroethyl)amino-phenylamino functionalized pyrazolo[4,3-
e]1,2,4-triazolo[1,5-c]pyrimidine derivatives: irreversible antagonists at the human A₃
adenosine receptor and molecular modeling studies. Baraldi, Pier Giovanni; Cacciari,
Barbara; Moro, Stefano; Romagnoli, Romeo; Ji, Xiao-duo; Jacobson, Kenneth A.; Gessi,
Stefania; Borea, Pier Andrea; Spalluto, Giampiero. Dipartimento di Scienze
Farmaceutiche, Universita degli Studi di Ferrara, Ferrara, Italy. Journal of Medicinal
Chemistry (2001), 44(17), 2735-2742.
U.S. Pat. No. 6,686,366
Involvement of A₃ receptors in the potentiation by adenosine of the inhibitory effect of
theophylline on human eosinophil degranulation: possible novel mechanism of the anti-
inflammatory action of theophylline. Ezeamuzie, C. I. Faculty of Medicine, Department
of Pharmacology and Toxicology, Kuwait University, Safat, Kuwait. Biochemical
Pharmacology (2001), 61(12), 1551-1559.
Receptor subtypes mediating adenosine-induced dilation of cerebral arterioles. Ngai, Al C.;
Coyne, Ellicia F.; Meno, Joseph R.; West, G. Alexander; Winn, H. Richard.
Department of Neurological Surgery, School of Medicine, University of Washington,
Seattle, WA, USA. American Journal of Physiology (2001), 280(5, Pt. 2), H2329-H2335.
Pyrazolo[4,3-e]1,2,4-triazolo[1,5-c]pyrimidine derivatives: a new pharmacological tool
for the characterization of the human A₃ adenosine receptor. Baraldi, Pier Giovanni;
Cacciari, Barbara; Romagnoli, Romeo; Spalluto, Giampiero; Varani, Katia; Gessi,
Stefania; Merighi, Stefania; Borea, Pier Andrea. Dipartimento di Scienze Farmaceutiche,
Universita degli Studi di Ferrara, Ferrara, Italy. Drug Development Research (2001),
52(1/2), 406-415.
Adenosine-enhanced ischemic preconditioning: adenosine receptor involvement during
ischemia and reperfusion. McCully, James D.; Toyoda, Yoshiya; Uematsu, Masahisa;
Stewart, Robert D.; Levitsky, Sidney. Division of Cardiothoracic Surgery, Beth Israel
Deaconess Medical Center and Harvard Medical School, Boston, MA, USA. American
Journal of Physiology (2001), 280(2, Pt. 2), H591-H602.
The adenosine A₃ receptor and its ligands. Van Muijlwijk-Koezen, Jacqueline E.;
Timmerman, Henk; Ijzerman, Adriaan P. Leiden/Amsterdam Center for Drug Research,
Division of Medicinal Chemistry, Department of Pharmacochemistry, Vrije Universiteit,
Amsterdam, Neth. Progress in Medicinal Chemistry (2001), 38 61-113.
WO 2001019360 A2
Cardioprotective effects of adenosine A₁ and A₃ receptor activation during hypoxia in
isolated rat cardiac myocytes. Safran, Noam; Shneyvays, Vladimir; Balas, Nissim;
Jacobson, Kenneth A.; Nawrath, Hermann; Shainberg, Asher. Gonda (Goldschmied)
Medical Diagnostic Research Center, Faculty of Life Sciences, Bar-Ilan University,
Ramat-Gan, Israel. Molecular and Cellular Biochemistry (2001), 217(1&2), 143-152.
Thiazole and thiadiazole analogs as a novel class of adenosine receptor antagonists. van
Muijlwijk-Koezen, Jacqueline E.; Timmerman, Hendrik; Vollinga, Roeland C.; von
Kuenzel, Jacobien Frijtag; de Groote, Miriam; Visser, Sven; IJzerman, Adriaan P.
Department of Pharmacochemistry Division of Medicinal Chemistry Leiden/Amsterdam
Center for Drug Research, Vrije Universiteit, Amsterdam, Neth. Journal of Medicinal
Chemistry (2001), 44(5), 749-762.
New potent and selective human adenosine A₃ receptor antagonists. Baraldi, P. G.;
Borea, P. A. Dipartimento di Scienze Farmaceutiche, Universita degli Studi di Ferrara,
Ferrara, Italy. Trends in Pharmacological Sciences (2000), 21(12), 456-459.
Pyrazolo[4,3-e]1,2,4-triazolo[1,5-c]pyrimidine Derivatives as Highly Potent and
Selective Human A₃ Adenosine Receptor Antagonists: Influence of the Chain at the N8
Pyrazole Nitrogen. Baraldi, Pier Giovanni; Cacciari, Barbara; Romagnoli, Romeo;
Spalluto, Giampiero; Moro, Stefano; Klotz, Karl-Norbert; Leung, Edward; Varani, Katia;
Gessi, Stefania; Merighi, Stefania; Borea, Pier Andrea. Dipartimento di Scienze
Farmaceutiche and Dipartimento di Medicina Clinica e Sperimentale-Sezione di
Farmacologia, Universita degli Studi di Ferrara, Ferrara, Italy. Journal of Medicinal
Chemistry (2000), 43(25), 4768-4780.
Pyran template approach to the design of G protein-coupled receptor antagonists.
Jacobson, Kenneth A.; Li, An-Hu; Kim, Hak Sung; Ji, Xiao-duo. Molecular Recognition
Section, Laboratory of Bioorganic Chemistry, National Institute of Diabetes, Digestive
and Kidney Disease, National Institutes of Health, Bethesda, MD, USA. Abstracts of
Papers, 220th ACS National Meeting, Washington, DC, United States, Aug. 20-24,
2000 (2000), MEDI-263.
7-Deazaadenines Bearing Polar Substituents: Structure-Activity Relationships of New A1
and A₃ Adenosine Receptor Antagonists. Hess, Sonja; Mueller, Christa E.; Frobenius,
Wolfram; Reith, Ulrike; Klotz, Karl-Norbert; Eger, Kurt. Pharmaceutical Chemistry
Institute of Pharmacy, University of Leipzig, Leipzig, Germany. Journal of Medicinal
Chemistry (2000), 43(24), 4636-4646.
WO 2000064894 A1
Insights into adenosine A₁ and A₃ receptors function: cardiotoxicity and cardioprotection.
Shneyvays, Vladimir; Safran, Noam; Halili-Rutman, Irit; Shainberg, Asher. Faculty of
Life Sciences, Bar-Ilan University, Ramat-Gan, Israel. Drug Development Research
(2000), 50(3/4), 324-337.
WO 2000044756 A1
Synthesis and structure-activity relationships of a new set of 2-arylpyrazolo[3,4-
c]quinoline derivatives as adenosine receptor antagonists. Colotta, Vittoria; Catarzi,
Daniela; Varano, Flavia; Cecchi, Lucia; Filacchioni, Guido; Martini, Claudia; Trincavelli,
Letizia; Lucacchini, Antonio. Dipartimento di Scienze Farmaceutiche, Universita' di
Firenze, Florence, Italy. Journal of Medicinal Chemistry (2000), 43(16), 3118-3124.
Synthesis and preliminary biological evaluation of [3H]-MRE 3008-F20: the first high
affinity radioligand antagonist for the human A₃ adenosine receptors. Baraldi, Pier
Giovanni; Cacciari, Barbara; Romagnoli, Romeo; Varani, Katia; Merighi, Stefania;
Gessi, Stefania; Borea, Pier Andrea; Leung, Edward; Hickey, Sarah L.; Spalluto,
Giampiero. Dipartimento di Scienze Farmaceutiche, Universita di Ferrara, Ferrara, Italy.
Bioorganic & Medicinal Chemistry Letters (2000), 10(12), 1403.
Patent evaluation: a novel adenosine receptor pharmacophore. Novartis AG, Expert
Opinion on Therapeutic Patents (2000), 10(5), 729-732.
Development of a selective adenosine A₁ receptor antagonist: Use of yeast functional
assays. Witter, David J.; McKibben, Bryan; Sherman, Dan; Werner, Doug; Zhang,
Hesheng; Castelhano, Arlindo L. Chemistry Department, OSI Pharmaceuticals,
Tarrytown, NY, USA. Book of Abstracts, 219th ACS National Meeting, San Francisco,
CA, March 26-30, 2000 (2000), MEDI-217. Publisher: American Chemical Society,
Washington, D.C.
Isoquinoline and Quinazoline Urea Analogues as Antagonists for the Human Adenosine
A3 Receptor. Van Muijlwijk-Koezen, Jacqueline E.; Timmerman, Henk; Van der Goot,
Henk; Menge, Wiro M. P. B.; Von Kuenzel, Jacobien Frijtag; De Groote, Miriam;
IJzerman, Adriaan P. Leiden/Amsterdam Center for Drug Research Division of
Medicinal Chemistry Department of Pharmacochemistry, Vrije Universiteit, Amsterdam,
Neth. Journal of Medicinal Chemistry (2000), 43(11), 2227-2238.
[3H]MRE 3008F20: a novel antagonist radioligand for the pharmacological and
biochemical characterization of human A₃ adenosine receptors. Varani, Katia; Merighi,
Stefania; Gessi, Stefania; Klotz, Karl-Norbert; Lfung, Edward; Baraldi, Pier Giovanni;
Cacciari, Barbara; Romagnoli, Romeo; Spalluto, Giampiero; Borfa, Pier Andrea.
Department of Clinical and Experimental Medicine, Pharmacology Unit, University of
Ferrara, Italy. Molecular Pharmacology (2000), 57(5), 968-975.
A3 adenosine receptor ligands: history and perspectives. Baraldi, Pier Giovanni;
Cacciari, Barbara; Romagnoli, Romeo; Merighi, Stefania; Varani, Katia; Borea, Pier
Andrea; Spalluto, Giampiero. Dipartimento di Scienze Farmaceutiche, Universita di
Ferrara, Ferrara, Italy. Medicinal Research Reviews (2000), 20(2), 103-128.
Pyran template approach to the design of novel A₃ adenosine reantagonists. Li,
An-Hu; Ji, Xiao-Duo; Kim, Hak Sung; Melman, Neli; Jacobson, Kenneth A. Molecular
Recognition Section, Laboratory of Bioorganic Chemistry, National Institute of Diabetes,
Digestive and Kidney Diseases, National Institutes of Health, Bethesda, MD, USA. Drug
Development Research (1999), 48(4), 171-177.
Synthesis and preliminary biological evaluation of [3H]-MRE 3008-F20: the first high
affinity radioligand antagonist for the human A₃ adenosine receptors. Baraldi, Pier
Giovanni; Cacciari, Barbara; Romagnoli, Romeo; Varani, Katia; Merighi, Stefania;
Gessi, Stefania; Borea, Pier Andrea; Leung, Edward; Hickey, Sarah L.; Spalluto,
Giampiero. Dipartimento di Scienze Farmaceutiche, Universita di Ferrara, Ferrara, Italy.
Bioorganic & Medicinal Chemistry Letters (2000), 10(3), 209-211.
1,2,4-Triazolo[4,3-a]quinoxalin-1-one: A Versatile Tool for the Synthesis of Potent and
Selective Adenosine Receptor Antagonists. Colotta, Vittoria; Catarzi, Daniela; Varano,
Flavia; Cecchi, Lucia; Filacchioni, Guido; Martini, Claudia; Trincavelli, Letizia;
Lucacchini, Antonio. Dipartimento di Scienze Farmaceutiche, Universita' di Firenze,
Florence, Italy. Journal of Medicinal Chemistry (2000), 43(6), 1158-1164.
U.S. Pat. No. 6,528,516
U.S. Pat. No. 6,376,521
Pyrazolo[4,3-e]-1,2,4-triazolo[1,5-c]pyrimidine derivatives as highly potent and selective
human A3 adenosine receptor antagonists. Baraldi, Pier Giovanni; Cacciari, Barbara;
Romagnoli, Romeo; Spalluto, Giampiero; Klotz, Karl-Norbert; Leung, Edward; Varani,
Katia; Gessi, Stefania; Merighi, Stefania; Borea, Pier Andrea. Dipartimento di Scienze
Farmaceutiche and Dipartimento di Medicina Clinica e Sperimentale-Sezione di
Farmacologia, Universita degli Studi di Ferrara, Ferrara, Italy. Journal of Medicinal
Chemistry (1999), 42(22), 4473-4478.
Selective A₃ Adenosine Receptor Antagonists: Water-Soluble 3,5-Diacyl-1,2,4-
trialkylpyridinium Salts and Their Oxidative Generation from Dihydropyridine
Precursors. Xie, Rongyuan; Li, An-Hu; Ji, Xiao-Duo; Melman, Neli; Olah, Mark E.;
Stiles, Gary L.; Jacobson, Kenneth A. Molecular Recognition Section Laboratory of
Bioorganic Chemistry National Institute of Diabetes Digestive and Kidney Diseases,
National Institutes of Health, Bethesda, MD, USA. Journal of Medicinal Chemistry
(1999), 42(20), 4232-4238.
Chiral Resolution and Stereospecificity of 6-Phenyl-4-phenylethynyl-1,4-
dihydropyridines as Selective A₃ Adenosine Receptor Antagonists. Jiang, Ji-long; Li,
An-Hu; Jang, Soo-Yeon; Chang, Louis; Melman, Neli; Moro, Stefano; Ji, Xiao-duo;
Lobkovsky, Emil B.; Clardy, Jon C.; Jacobson, Kenneth A. Molecular Recognition
Section Laboratory of Bioorganic Chemistry, National Institute of Diabetes Digestive and
Kidney Diseases National Institutes of Health, Bethesda, MD, USA. Journal of
Medicinal Chemistry (1999), 42(16), 3055-3065.
JP 11158073 A2
Functionalized Congeners of 1,4-Dihydropyridines as Antagonist Molecular Probes for
A3 Adenosine Receptors. Li, An-Hu; Chang, Louis; Ji, Xiao-duo; Melman, Neli;
Jacobson, Kenneth A. Molecular Recognition Section Laboratory of Bioorganic
Chemistry, National Institute of Diabetes Digestive and Kidney Diseases National
Institutes of Health, Bethesda, MD, USA. Bioconjugate Chemistry (1999), 10(4), 667-677.
WO 9921555 A2
A₃ adenosine receptors regulate C1-channels of nonpigmented ciliary epithelial cells.
Mitchell, Claire H.; Peterson-Yantorno, Kim; Carre, David A.; McGlinn, Alice M.; Coca-
Prados, Miguel; Stone, Richard A.; Civan, Mortimer M. Departments of Physiology,
Ophthalmology, and Medicine, University of Pennsylvania School of Medicine,
Philadelphia, PA, USA. American Journal of Physiology (1999), 276(3, Pt. 1), C659-C666.
CODEN: AJPHAP ISSN: 0002-9513.
Medicinal chemistry of the human adenosine A₃ receptor. Van Tilburg, Erica W.; Van
Muijlwijk-Koezen, Jacqueline E.; Ilzerman, Adriaan P. Leiden/Amsterdam Center for
Drug Research, Division of Medicinal Chemistry, Gorlaeus Laboratories,
Rijksuniversiteit Leiden, Leiden, Neth. Drug Development Research (1998), 45(3/4),
182-189.
Characterization of potent ligands at human recombinant adenosine receptors. Cristalli,
Gloria; Camaioni, Emidio; Costanzi, Stefano; Vittori, Sauro; Volpini, Rosaria; Klotz,
Karl-Norbert. Dipartimento di Scienze Chimiche, Universita di Camerino, Camerino,
Italy. Drug Development Research (1998), 45(3/4), 176-181.
A₃ adenosine receptors: protective vs. damaging effects identified using novel agonists
and antagonists. Jacobson, Kenneth A.; Moro, Stefano; Kim, Yong-Chul; Li, An-Hu.
Molecular Recognition Section, Laboratory of Bioorganic Chemistry, National Institute
of Diabetes, Digestive and Kidney Diseases, National Institutes of Health, Bethesda, MD,
USA. Drug Development Research (1998), 45(3/4), 113-124.
Synthesis, CoMFA Analysis, and Receptor Docking of 3,5-Diacyl-2,4-Dialkylpyridine
Derivatives as Selective A₃ Adenosine Receptor Antagonists. Li, An-Hu; Moro, Stefano;
Forsyth, Nancy; Melman, Neli; Ji, Xiao-duo; Jacobson, Kenneth A. Molecular
Recognition Section Laboratory of Bioorganic Chemistry, National Institute of Diabetes
Digestive and Kidney Diseases, National Institutes of Health, Bethesda, MD, USA.
Journal of Medicinal Chemistry (1999), 42(4), 706-721.
Molecular Modeling Studies of Human A₃ Adenosine Antagonists: Structural Homology
and Receptor Docking. Moro, Stefano; Li, An-Hu; Jacobson, Kenneth A. Molecular
Recognition Section Laboratory of Bioorganic Chemistry National Institute of Diabetes
Digestive and Kidney Diseases, National Institutes of Health, Bethesda, MD, USA.
Journal of Chemical Information and Computer Sciences (1998), 38(6), 1239-1248.
A novel class of adenosine A₃ receptor ligands. 1. 3-(2-Pyridinyl)isoquinoline
derivatives. van Muijlwijk-Koezen, Jacqueline E.; Timmerman, Henk; Link, Regina; vander
Goot, Henk; IJzerman, Adriaan P. Division of Medicinal Chemistry
Leiden/Amsterdam Center for Drug Research, Department of Pharmacochemistry Vrije
Universiteit, Amsterdam, Neth. Journal of Medicinal Chemistry (1998), 41(21), 3987-3993.
Molecular modeling studies of human A₃ adenosine antagonists: Structure similarity and
receptor docking. Moro, Stefano; Li, An-Hu; Jacobson, Kenneth A. Lab. Bioorganic
Chem., NIDDK, NIH, Bethesda, MD, USA. Book of Abstracts, 216th ACS National
Meeting, Boston, August 23-27 (1998), MEDI-094. Publisher: American Chemical
Society, Washington, D.C.
Further pharmacological characterization of the adenosine receptor subtype mediating
inhibition of oxidative burst in human isolated neutrophils. Hannon, J. P.; Bray-French, K. M.;
Phillips, R. M.; Fozard, J. R. Novartis Pharma Ltd., Basel, Switz. Drug
Development Research (1998), 43(4), 214-224.
Structure-Activity Relationships and Molecular Modeling of 3,5-Diacyl-2,4-
dialkylpyridine Derivatives as Selective A3 Adenosine Receptor Antagonists. Li, An-
Hu; Moro, Stefano; Melman, Neli; Ji, Xiao-duo; Jacobson, Kenneth A. Molecular
Recognition Section, Laboratory of Bioorganic Chemistry, National Institute of Diabetes
Digestive and Kidney Diseases, National Institutes of Health, Bethesda, MD, USA.
Journal of Medicinal Chemistry (1998), 41(17), 3186-3201.
Derivatives of the Triazoloquinazoline Adenosine Antagonist (CGS 15943) Having High
Potency at the Human A_{2B} and A₃ Receptor Subtypes. Kim, Yong-Chul; de Zwart,
Maarten; Chang, Louis; Moro, Stefano; von Kuenzel, Jacobien K.; Melman, Neli;
IJzerman, Ad P.; Jacobson, Kenneth A. Molecular Recognition Section, Laboratory of
Bioorganic Chemistry, National Institute of Diabetes Digestive and Kidney Diseases,
National Institutes of Health, Bethesda, MD, USA. Journal of Medicinal Chemistry
(1998), 41(15), 2835-2845.
Binding affinity of adenosine receptor agonists and antagonists at human cloned A₃
adenosine receptors. Varani, K.; Cacciari, B.; Baraldi, P. G.; Dionisotti, S.; Ongini, E.;
Borea, P. A. Dip. Med. Clin. Sperimentale-Sezione Farmacologia, Univ. Studi Ferrara,
Ferrara, Italy. Life Sciences (1998), 63(5), PL81-PL87.
A physiological role of the adenosine A₃ receptor: sustained cardioprotection. Liang,
Bruce T.; Jacobson, Kenneth A. Department of Medicine, Cardiovascular Division,
University of Pennsylvania Medical Center, Philadelphia, PA, USA. Proceedings of the
National Academy of Sciences of the United States of America (1998), 95(12), 6995-6999.
Adenosine A₃ receptors: novel ligands and paradoxical effects. Jacobson, Kenneth A.
Molecular Recognition Section, Lab. Bioorganic Chem., National Inst. Diabetes,
Digestive and Kidney Diseases, national Inst. Health, Bethesda, MD, USA. Trends in
Pharmacological Sciences (1998), 19(5), 184-191.
New substituted 9-alkylpurines as adenosine receptor ligands. Camaioni, Emidio;
Costanzi, Stefano; Vittori, Sauro; Volpini, Rosaria; Klotz, Karl-Norbert; Cristalli, Gloria.
Dipartimento di Scienze Chimiche, Universita di Camerino, Camerino, Italy. Bioorganic
& Medicinal Chemistry (1998), 6(5), 523-533.
U.S. Pat. No. 6,306,847
Novel selective non-xanthine A₃ adenosine receptor antagonists. Jacobson, Marlene A.
Department Pharmacology, Merck Research Labs, West Point, PA, USA. Book of
Abstracts, 215th ACS National Meeting, Dallas, March 29-April 2 (1998), MEDI-095.
Publisher: American Chemical Society, Washington, D.C.
Flavonoid Derivatives as Adenosine Receptor Antagonists: A Comparison of the
Hypothetical Receptor Binding Site Based on a Comparative Molecular Field Analysis
Model. Moro, Stefano; van Rhee, A. Michiel; Sanders, Lawrence H.; Jacobson, Kenneth A.
Molecular Recognition Section Laboratory of Bioorganic Chemistry National
Institute of Diabetes Digestive and Kidney Diseases, National Institutes of Health,
Bethesda, MD, USA. Journal of Medicinal Chemistry (1998), 41(1), 46-52.
Ca²⁺ dependence of the response of three adenosine type receptors in rat hepatocytes.
Guinzberg, Raquel; Diaz-Cruz, Antonio; Uribe, Salvador; Pina, Enrique. Apdo. Postal,
School of Medicine, Department of Biochemistry, Universidad Nacional Autonoma de
Mexico, 04510 Mexico DF, Mex. European Journal of Pharmacology (1997), 340(2/3),
243-247.
JP 09291089 A2
Pharmacological characterization of novel A₃ adenosine receptor-selective antagonists.
Jacobson, Kenneth A.; Park, Kyung-Sun; Jiang, Ji-Long; Kim, Yong-Chul; Olah, Mark E.;
Stiles, Gary L.; Ji, Xiao-Duo. Molecular Recognition Section, LBC, NIDDK,
National Institutes of Health, Bethesda, MD, USA. Neuropharmacology (1997), 36(9),
1157-1165.
U.S. Pat. No. 6,066,642
Adenosine A₃ receptor agonists inhibit murine macrophage tumor necrosis factor-d
production in vitro and in vivo. Bowlin, Terry L.; Borcherding, David R.; Edwards, Carl K.,
III; McWhinney, Charlene D. Department of Immunology, Marion Merrell Dow
Research Institute, Marion Merrell Dow Pharmaceuticals, Cincinnati, OH, USA. Cellular
and Molecular Biology (Paris) (1997), 43(3), 345-349.
Highly potent and selective human adenosine A₃ receptor antagonists:
Triazoloquinazoline derivatives. Kim, Y. -C.; Ji, X. -D.; Olah, M. E.; Stiles, G. L.;
Jacobson, K. A. Lab. Bioorg. Chem., Nat. Inst. Health, Bethesda, MD, USA. Book of
Abstracts, 213th ACS National Meeting, San Francisco, April 13-17 (1997), MEDI-042.
Publisher: American Chemical Society, Washington, D.C.
Adenosine receptor antagonists: structures and potential therapeutic applications.
Mueller, C. E.; Stein, B. Inst. Pharmazie Lebensmittelchemie, Univ. Wuerzburg,
Wuerzburg, Germany. Current Pharmaceutical Design (1996), 2(5), 501-530.
*U.S. Pat. No. 6,040,296
Cloning, expression and pharmacological characterization of rabbit adenosine A₁ and A₃
receptors. Hill, Roger J.; Oleynek, Joseph J.; Hoth, Christopher F.; Ravi Kiron, M. A.;
Weng, Weifan; Wester, Ronald T.; Tracey, W. Ross; Knight, Delvin R.; Buchholz, R.
Allan; Kennedy, Scott P. Dep. Cardiovascular, Pfizer Inc., Groton, CT, USA. Journal of
Pharmacology and Experimental Therapeutics (1997), 280(1), 122-128.
6-Phenyl-1,4-Dihydropyridine Derivatives as Potent and Selective A₃ Adenosine
Receptor Antagonists. Jiang, Ji-long; van Rhee, A. Michiel; Melman, Neli; Ji, Xiao-duo;
Jacobson, Kenneth A. Laboratory of Bioorganic Chemistry, National Institute of
Diabetes Digestive and Kidney Diseases, Bethesda, MD, USA. Journal of Medicinal
Chemistry (1996), 39(23), 4667-4675.
Derivatives of the Triazoloquinazoline Adenosine Antagonist (CGS15943) Are Selective
for the Human A₃ Receptor Subtype. Kim, Yong-Chul; Ji, Xiao-duo; Jacobson, Kenneth A.
Laboratory of Bioorganic Chemistry, National Institute of Diabetes Digestive and
Kidney Diseases, Bethesda, MD, USA. Journal of Medicinal Chemistry (1996), 39(21),
4142-4148.
Activation of adenosine A₃ receptors on macrophages inhibits tumor necrosis factor-d
McWhinney, Charlene D.; Dudley, Mark W.; Bowlin, Terry L.; Peet, Norton P.; Schook,
Larry; Bradshaw, Marita; De, Mamata; Borcherding, David R.; Edwards, Carl K., III.
Department of Immunology, Marion Merrell Dow Research Institute, Marion Merrell
Dow Pharmaceuticals, Inc., 2110 East Galbraith Road, Cincinnati, USA. European
Journal of Pharmacology (1996), 310(2/3), 209-216.
Novel adenosine antagonists: A₃ receptor-selective flavonoid and dihydropyridine
derivatives. Jacobson, Kenneth A.; Van Rhee, A. Michiel; Jiang, Ji-Long; Ji, Xiao-duo;
Karton, Yishai; Von Lubitz, Dag K. J. E.; Olah, Mark E.; Stiles, Gary L. Laboratory
Bioorganic Chemistry, NIDDK, NIH, Bethesda, MD, USA. Book of Abstracts, 212th
ACS National Meeting, Orlando, FL, August 25-29 (1996), MEDI-099. Publisher:
American Chemical Society, Washington, D.C.
Synthesis and biological activities of flavonoid derivatives as A₃ adenosine receptor
antagonists. Karton, Yishai; Jiang, Ji-long; Ji, Xiao-duo; Melman, Neli; Olah, Mark E.;
Stiles, Gary L.; Jacobson, Kenneth A. Laboratory of Bioorganic Chemistry, National
Institute of Diabetes Digestive and Kidney Diseases, Bethesda, MD, USA. Journal of
Medicinal Chemistry (1996), 39(12), 2293-2301.
U.S. Pat. No. 5,646,156
WO 9511681
Structure-Activity Relationships of 9-Alkyladenine and Ribose-Modified Adenosine
Derivatives at Rat A3 Adenosine Receptors. Jacobson, Kenneth A.; Siddiqi, Suhaib M.;
Olah, Mark E.; Ji, Xiao-duo; Melman, Neli; Bellamkonda, Kamala; Meshulam, Yacov;
Stiles, Gary L.; Kim, Hea O. Laboratory of Bioorganic Chemistry, National Institute of
Diabetes and Digestive and Kidney Diseases, Bethesda, MD, USA. Journal of Medicinal
Chemistry (1995), 38(10), 1720-35.
Evidence that the adenosine A₃ receptor may mediate the protection afforded by
preconditioning in the isolated rabbit heart. Liu, Guang S.; Richards, Steven C.; Olsson,
Ray A.; Mullane, Kevin; Walsh, Robert S.; Downey, James M. Department Physiology
MSB 3024, University South Alabama, Mobile, AL, USA. Cardiovascular Research
(1994), 28(7), 1057-61.
Cloned adenosine A₃ receptors: pharmacological properties, species differences and
receptor functions. Linden, Joel. Health Sci. Cent., Univ. Virginia, Charlottesville, VA,
USA. Trends in Pharmacological Sciences (1994), 15(8), 298-306.
Species differences in ligand affinity at central A₃-adenosine receptors. Ji, Xiao-Duo;
von Lubitz, Dag; Olah, Mark E.; Stiles, Gary L.; Jacobson, Kenneth A. Laboratory of
Bioorganic Chemistry, National Institute of Diabetes, Digestive and Kidney Diseases,
Bethesda, MD, USA. Drug Development Research (1994), 33(1), 51-9.
U.S. Pat. No. 5,443,836
Molecular cloning and characterization of the human A3 adenosine receptor. Salvatore,
Christopher A.; Jacobson, Marlene A.; Taylor, Heidi E.; Linden, Joel; Johnson, Robert G.
Dep. Pharmacol., Merck Res. Lab., West Point, PA, USA. Proceedings of the National
Academy of Sciences of the United States of America (1993), 90(21), 10365-9.
U.S. Pat. No. 20040186133
U.S. Pat. No. 20030087845
U.S. Pat. No. 20020094974
U.S. Pat. No. 6,387,913
U.S. Pat. No. 6,288,070
U.S. Pat. No. 5,573,772
WO 200350241 A2-A3
WO 200285308 A2-A3
WO 200283152 A1
WO 200270532 A2-A3
WO 200266020 A2-A3
WO 200257267 A1
WO 200139777 A1
WO 200051621 A1
WO 200003741 A2
WO 9962518 A1
WO 9920284 A1
WO 9421195 A1
JP 2004135657 A

The A₃ receptor antagonists of the invention are particularly useful for prevention, treatment, and/or management of cancer, for example, as a single agent therapy or in combination with other modes of therapy for cancer. In preferred embodiments, the invention encompasses methods of treatment, prevention, or management of cancers which are A₃ receptor expressing cancers including, but not limited to, human leukemia, melanoma, pancreatic carcinoma, ovarian carcinoma, breast carcinoma, prostrate carcinoma, colon carcinoma, lung carcinoma, malignant melanomas, histiocytic lymphoma, and some forms of astrocytoma cells. In other preferred embodiments, the invention encompasses methods of treatment, prevention, or management which have high level of expression of HIF-1α including, but not limited to, cervical cancer (early stage), lung cancer (non-small cell lung carcinoma), breast cancer (including lymph node positive breast cancer and lymph node negative breast cancer), oligodendroglioma, orpharyngeal squamous cell carcinoma, ovarian cancer, oesophageal cancer, endometrial cancer, head and neck cancer, gastrointestinal stromal tumor of the stomach The methods of the invention are particularly useful in cancer therapy for providing tissue selectivity, such that the biological effect is observed in tumoral hypoxic cells, where high adenosine concentrations has resulted in increased HIF-1α accumulation.

The methods and compositions of the invention comprising A₃ receptor antagonists (or A₁ receptor antagonists or a combination thereof) are particularly useful for the treatment, inhibition or regression of solid tumors. As used herein, “solid tumors” refer to a locus of tumor cells where the majority of the cells are tumor cells or tumor-associated cells, including but not limited to laryngeal tumors, brain tumors, and other tumors of the head and neck; colon, rectal and prostate tumors; breast and thoracic solid tumors; ovarian and uterine tumors; tumors of the esophagus, stomach, pancreas and liver; bladder and gall bladder tumors; skin tumors such as melanomas. Moreover, the tumors encompassed within the invention can be either primary or a secondary tumor resulting from metastasis of cancer cells elsewhere in the body to the chest.

The invention encompasses use of the A₃ or A₁ receptor antagonist compounds of the invention in inhibiting tumor growth in a mammal, including humans. The invention encompasses methods for administering an effective amount of adenosine A₁ or A₃ antagonists, preferably, A₃ antagonists sufficient to inhibit the ability of adenosine to protect tumor cells.

In other embodiments, the present invention encompasses methods for treatment, prevention and/or management of cancer comprising administering a compound of the invention including A₃ and/or A₁ receptor antagonists used in combination with other therapeutic and/or prophylactic agents, e.g., cytotoxic agents. The inventors have surprisingly found that adenosine A₃ receptor antagonists synergistically enhance cytotoxic treatment and counter some forms of multi-drug resistance. Although not intending to be bound by a particular mechanism of action, the combination therapies of the invention will attack the tumor cell directly, inhibit growth of new blood vessels around the tumor cell, and, by virtue of the adenosine A₃ antagonists, inhibit the ability of the cell to survive without the growth of new blood vessels. Inventors have discovered that high affinity adenosine A₃ receptor antagonists are useful as enhancers for many chemotherapeutic treatments of adenosine A₃ receptor expressing cancers. Surprisingly, high affinity adenosine A₃ receptor antagonists also counter P-glycoprotein (P-gp) effuse pump multi-drug resistance (MDR). Finally, high affinity adenosine A₃ receptor antagonists are helpful in reducing or ameliorating side effects of cytoxic agents especially taxane induced hypersensitivity. For example, lower doses of taxane may be used once used in combination with an adenosine A₃ receptor antagonist of the invention.

In other embodiments, the present invention encompasses the use of high affinity adenosine A₃ receptor antagonists for enhancing chemotherapeutic treatment of cancers expressing adenosine A₃ receptors and countering multi-drug resistance in cancers expressing P-glycoprotein or MRP. In preferred embodiments, adenosine A₃ receptor antagonists are administered before or during administration of a taxane family, vinca alkaloid, camptothecin or antibiotic compound. Preferred high affinity A₃ receptor antagonists include compounds of the general formulas IIA, IIB, IIC and IID described herein, vide infra.

The present invention encompasses therapies which involve administering one or more of the compounds of the invention, to an animal, preferably a mammal, and most preferably a human, for preventing, treating, or ameliorating symptoms associated with a cancer, a disease or disorder associated with hypoxia-inducible factor 1-α (HIF-1α).

The invention further provides a pharmaceutical composition comprising a therapeutically or prophylactically effective amount of a compound of the invention that specifically binds an A₁ or A₃ receptor and a pharmaceutically acceptable carrier Preferably the pharmaceutical formulation includes an adenosine A₃ receptor antagonist and one or more excipients. The formulations can also include other anti-tumor agents, including cytotoxic agents and other anti-angiogenesis agents, including adenosine A_{2A} antagonists, such as ZM241385 and SCH 5861, adenosine A_{2B} antagonists, such as MRE-20290-F20 and AS-16, and anti-VEGF antibodies, including humanized and chimeric antibodies. In a preferred embodiment, the composition includes an effective amount to inhibit tumor growth of an adenosine A₃ receptor antagonist, a cytotoxic agent, and an anti-angiogenesis agent.

4.1 Definitions

As used herein, a compound is an “antagonist of an adenosine A₃ receptor” if it is able to prevent the action due to an agonist and is able to displace [¹²⁵I]-AB-MECA in a competitive binding assay.

As used herein, a compound is selective for the A₃ receptor if its affinity at the A₃ receptor is greater than its affinity at the A₁, A_{2A} and A_{2B} receptors. Preferably, the ratio of A₁/A₃ and A₂/A₃ activity is greater than about 50, preferably between 50 and 100, and more preferably, greater than about 100. Since the pharmacology at the A₃ receptor varies between species, especially between rodent A₃ and human A₃ receptors, it is important to determine the selectivity of the A₃ compounds in human adenosine receptors. The same holds true for adenosine A₁ and A_{2A} receptors in terms of whether they are selective.

As used herein “Adenosine A₁ Antagonists” carry their common ordinary meaning. Adenosine A₁ antagonists are well known to those of skill in the art. Some of the more well known compounds include CPX, CVT 124, FK 352, and ILF 117, the structures of which are well known.

As used herein “Adenosine A₃ Antagonists” carry their common ordinary meaning. Adenosine A₃ antagonists are well known to those of skill in the art. Some of the more well known compounds include MRS 1097, MRS 1191 and MRS 1220 (commercially available from Research Biochemicals International, Natick Mass.). Other suitable antagonists include those disclosed in Jacobson et al., Neuropharmacology, 36:1157-1165 (1997); Yao et al., Biochem. Biophys. Res. Commun., 232:317-322 (1997); Kim et al., J. Med. Chem., 39(21):4142-4148 (1996); van Rhee et al., Drug Devel. Res., 37:131 (1996); van Rhee et al., J. Med. Chem., 39:2980-2989 (1996); Siquidi et al., Nucleosides, Nucleotides 15:693-718 (1996); van Rhee et al., J. Med. Chem., 39:398-406 (1996); Jacobson et al., Drugs of the Future, 20:689-699 (1995); Jacobson et al., J. Med. Chem., 38:1720-1735 (1995); Karton et al., J. Med. Chem., 39:2293-2301 (1996); Kohno et al., Blood, 88:3569-3574 (1996); Jiang et al., J. Med. Chem., 39:4667-4675 (1996); Yao et al., Biochem. Biophys. Res. Commun. 232:317-322 (1997); and Jiang et al., J. Med. Chem. 40:2596-2608 (1996), the contents of which are hereby incorporated by reference in their entireties. In addition, other suitable adenosine A₃ antagonists are disclosed in the references listed in Table 1, the disclosures of which are hereby incorporated by reference in their entireties.

As used herein, “elevation” of a measured level of HIF-1α or A₃ receptor relative to a standard level means that the amount or concentration of HIF-1α or A₃ receptor in a sample or subject is sufficiently greater in a subject or sample relative to the standard as detected by any method now known in the art or to be developed in the future for measuring HIF-1α or A₃ receptor levels. For example, elevation of the measured level relative to a standard level may be any statistically significant elevation detectable. Such an elevation in HIF-1α expression and/or activity may include, but is not limited to about a 10%, about a 20%, about a 40%, about an 80%, about a 2-fold, about a 4-fold, about an 10-fold, about a 20-fold, about a 50-fold, about a 100-fold, about a 2 to 20 fold, 2 to 50 fold, 2 to 100 fold, 20 to 50 fold, 20 to 100 fold, elevation, relative to the standard. The term “about” as used herein, refers to levels of elevation of the standard numerical value plus or minus 10% of the numerical value.

The term “standard level” or “background level” as used herein refers to a baseline amount of an HIF-1α level as determined in one or more normal subjects, i.e., a subject with no known history of past or current diseases, disorders or cancer. For example, a baseline may be obtained from at least one subject and preferably is obtained from an average of subjects (e.g., n=2 to 100 or more), wherein the subject or subjects have no prior history of diseases, disorders or cancer, especially no prior history of diseases associated with an aberrant level of expression and/or activity of HIF-1α. In the present invention, the measurement of HIF-1α level may be carried out using an HIF-1α probe or a HIF-1α activity assay (see Section 6).

As used herein, “enhancement” refers to a synergistic effect as determined from measurement of the enhancement factor, as defined below. In general, an enhancement factor of greater than one is considered synergistic. For example, if one of the compounds has little individual chemotherapeutic effect, an enhancement factor greater than one indicates a synergistic effect is occurring.

As used herein, the term “cancer” refers to a neoplasm or tumor resulting from abnormal uncontrolled growth of cells. As used herein, cancer explicitly includes, leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation. Non-cancer cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen.

As used herein, the term “A₃ expressing cancers” refers to human cancers that express the adenosine A₃ receptor or that otherwise comprise elevated concentrations of adenosine A₃ receptors. Elevated concentration is determined by comparison to normal, non-cancerous tissues of a similar cell type. Examples of A₃ expressing cancers include, without limitation, human leukemia, melanoma, pancreatic carcinoma, breast carcinoma, prostrate carcinoma, colon carcinoma, lung carcinomamalignant melanomas, histiocytic lymphoma, and some forms of astrocytoma cells.

As used herein the term HIF-1α expressing cancers refers to human cancer that express HIF-1α or that otherwise comprise elevated concentrations of HIF-1α. Elevated concentration is determined by comparison to normal, non-cancerous tissues of a similar cell type. Examples of HIF-1α expressing cancers include without limitation cervical cancer (early stage), melanoma, lung cancer (non-small cell lung carcinoma), breast cancer (including lymph node positive breast cancer and lymph node negative breast cancer), oligodendroglioma, orpharyngeal squamous cell carcinoma, ovarian cancer, oesophageal cancer, endometrial cancer, head and neck cancer, gastrointestinal stromal tumor of the stomach.

As used herein “hypoxia” or “ischemia” refers to any condition whereby the physiology of the tissue is compromised and/or blood supply in one or more tissues is compromised. These two terms may be used interchangeably. The condition also encompasses any reduction in partial pressure of O₂ in one or more tissues. The term “hypoxia” or “ischemia” encompasses any condition in which a cell, tissue or organ experiences a lack of oxygen or reduced blood flow.

As used herein, the terms “treat,” “treating” and “treatment” refer to the eradication, removal, modification, or control of primary, regional, or metastatic cancer tissue that results from the administration of one or more therapeutic agents. In certain embodiments, such terms refer to the minimizing or delaying the spread of cancer resulting from the administration of one or more therapeutic agents to a subject with such a disease. The term also refers to the eradication, reduction or amelioration of a symptom or disorder related to aberrant levels of HIF-1α level and/or activity.

As used herein, a “therapeutically effective amount” refers to that amount of the therapeutic agent sufficient to delay or minimize the spread of cancer. A therapeutically effective amount may also refer to the amount of the therapeutic agent that provides a therapeutic benefit in the treatment or management of a disease or disorder. Further, a therapeutically effective amount with respect to a therapeutic agent of the invention means that amount of therapeutic agent alone, or in combination with other therapies, that provides a therapeutic benefit in the treatment or management of a disease or disorder. Used in connection with an amount of a compound of the invention, the term can encompass an amount that improves overall therapy, reduces or avoids unwanted effects, or enhances the therapeutic efficacy of or synergizes with another therapeutic agent.

As used herein, the terms “prophylactic agent” and “prophylactic agents” refer to any agent(s) which can be used in the prevention of a disorder, or prevention of recurrence or spread of a disorder. A prophylactically effective amount may refer to the amount of prophylactic agent sufficient to prevent the recurrence or spread of hyperproliferative disease, particularly cancer, or the occurrence of such in a patient, including but not limited to those predisposed to hyperproliferative disease, for example those genetically predisposed to cancer or previously exposed to carcinogens. A prophylactically effective amount may also refer to the amount of the prophylactic agent that provides a prophylactic benefit in the prevention of disease. Further, a prophylactically effective amount with respect to a prophylactic agent of the invention means that amount of prophylactic agent alone, or in combination with other agents, that provides a prophylactic benefit in the prevention of disease. Used in connection with an amount of a compound of the invention, the term can encompass an amount that improves overall prophylaxis or enhances the prophylactic efficacy of or synergizes with another prophylactic agent, such as but not limited to a therapeutic antibody.

As used herein, the terms “manage,” “managing” and “management” refer to the beneficial effects that a subject derives from administration of a prophylactic or therapeutic agent, which does not result in a cure of the disease or diseases. In certain embodiments, a subject is administered one or more prophylactic or therapeutic agents to “manage” a disease so as to prevent the progression or worsening of the disease or diseases.

As used herein, the terms “prevent”, “preventing” and “prevention” refer to the methods to avert or avoid a disease or disorder or delay the recurrence or onset of one or more symptoms of a disorder in a subject resulting from the administration of a prophylactic agent.

As used herein, the term “in combination” refers to the use of more than one prophylactic and/or therapeutic agents. The use of the term “in combination” does not restrict the order in which prophylactic and/or therapeutic agents are administered to a subject with a disorder, e.g., hyperproliferative cell disorder, especially cancer. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject which had, has, or is susceptible to a disorder. The prophylactic or therapeutic agents are administered to a subject in a sequence and within a time interval such that the agent of the invention can act together with the other agent to provide an increased benefit than if they were administered otherwise. Any additional prophylactic or therapeutic agent can be administered in any order with the other additional prophylactic or therapeutic agents.

As used herein, the term “alkyl” refers to a monovalent straight, branched or cyclic saturated hydrocarbon group preferably having from 1 to 20 carbon atoms, more preferably 1 to 10 carbon atoms (“lower alkyl”) and most preferably 1 to 6 carbon atoms. This term is exemplified by groups such as methyl, ethyl, n-propyl, iso-propyl, -butyl, iso-butyl, n-hexyl, and the like. The terms “alkylene” and “lower alkylene” refer to divalent radicals of the corresponding alkane. Further, as used herein, other moieties having names derived from alkanes, such as alkoxyl, alkanoyl, alkenyl, cycloalkenyl, etc. when modified by “lower,” have carbon chains of ten or less carbon atoms. In those cases where the minimum number of carbons are greater than one, e.g., alkenyl (minimum of two carbons) and cycloalkyl, (minimum of three carbons), it is to be understood that “lower” means at least the minimum number of carbons.

As used herein, the term “substituted alkyl” refers to an alkyl group, preferably of from 1 to 10 carbon atoms (“substituted lower alkyl”), having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino aminoacyl, aminoacyloxy, oxyacylamino, cyano, halogen, hydroxyl, keto, thioketo, carboxyl, carboxylalkyl, thiol, thioalkoxy, substituted thioalkoxy, aryl, aryloxy, heteroaryl, heteroaryloxy, heterocyclic, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, aryl, substituted aryl, heteroaryl, substituted heteroaryl, heterocyclic, and substituted heterocyclic. As used herein, other moieties having the prefix “substituted” are intended to include one or more of the substituents listed above.

As used herein, “aralkyl” refers to an alkyl group with an aryl substituent. Binding is through the alkyl group. “Alkaryl” refers to an aryl group with an alkyl substituent, where binding is through the aryl group.

As used herein, the term “alkoxy” refers to the group “alkyl-O—”, where alkyl is as defined above. Preferred alkoxy groups include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

As used herein, the term “alkenyl” refers to an unsaturated, straight or branched hydrocarbon group preferably having from 2 to 10 carbon atoms and more preferably 2 to 6 carbon atoms and having at least 1 and preferably from 1-2 double bonds. Preferred alkenyl groups include ethenyl (—CH═CH₂), n-propenyl (—CH₂ CH═CH₂), iso-propenyl (—C(CH₃)═CH₂), and the like.

As used herein, the term “alkynyl” refers to an unsaturated, straight or branched hydrocarbon group preferably having from 2 to 10 carbon atoms and more preferably 2 to 6 carbon atoms and having at least 1 and preferably from 1-2 triple bonds.

As used herein, the term “acyl” refers to the groups alkyl-C(O)—, substituted alkyl-C(O)—, cycloalkyl-C(O)—, substituted cycloalkyl-C(O)—, aryl-C(O)—, substituted aryl-C(O)—, heteroaryl-C(O)— and heterocyclic-C(O)— where alkyl, substituted alkyl, cycloalkyl, substituted cycloalkyl, aryl, heteroaryl and heterocyclic are as defined herein.

As used herein, the term “aminoacyl” refers to the group —C(O)NR³³R³³ where each R³³ is independently hydrogen, alkyl, substituted alkyl, aryl, substituted aryl, heteroaryl, or heterocyclic wherein alkyl, substituted alkyl, aryl, heteroaryl and heterocyclic are as defined herein.

As used herein, the term “acylamino” refers to the group R³⁴—(O)—N(R³³)— where each R³⁴ is independently alkyl, substituted alkyl, cycloalkyl, substituted cycloalkyl, aryl, substituted aryl, heteroaryl, or heterocyclic wherein alkyl, substituted alkyl, cycloalkyl, substituted cycloalkyl, aryl, heteroaryl and heterocyclic are as defined herein. R³³ is independently hydrogen, alkyl, substituted alkyl, aryl, substituted aryl, heteroaryl, or heterocyclic wherein alkyl, substituted alkyl, aryl, heteroaryl and heterocyclic are as defined herein.

As used herein, the term “acyloxy” refers to the group R³⁴—C(O)— where each R³⁴ is independently alkyl, substituted alkyl, cycloalkyl, substituted cycloalkyl, aryl, substituted aryl, heteroaryl, or heterocyclic wherein alkyl, substituted alkyl, cycloalkyl, aryl, heteroaryl and heterocyclic are as defined herein.

As used herein the term “amino acid” means an alpha amino acid selected from those amino acids which naturally occur in proteins but without regard for specific stereochemical properties. The term “protected amino acid” means an amino acid of which the alpha amino group has been converted to a less reactive moiety, but a moiety which can be converted back to the amino group with relative ease. The terms “amino acid residue” and “amino acid moiety” are used synonymously herein.

As used herein, the term “aryl” refers to an unsaturated aromatic carbocyclic group of from 6 to 14 carbon atoms having a single ring (e.g., phenyl) or multiple condensed (fused) rings (e.g., naphthyl or anthryl). Preferred aryls include phenyl, naphthyl and the like. Unless otherwise constrained by the definition for the aryl substituent, such aryl groups can optionally be substituted with from 1 to 5 substituents and preferably 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, substituted amino, aminoacyl, acyloxy, acylamino, alkaryl, aralkyl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, alkylthio, —SO-alkyl, —SO-substituted alkyl, —SO-aryl, —SO-heteroaryl, —SO₂-alkyl, —SO₂ substituted alkyl, —SO₂-aryl, —SO₂-heteroaryl, trihalomethyl. Preferred substituents include alkyl, alkoxy, halo, cyano, nitro, trihalomethyl, and thioalkoxy.

As used herein, the term “cycloalkyl” refers to cyclic alkyl groups of from 3 to 12 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantyl, and the like.

As used herein, the terms “halo” or “halogen” refer to fluoro, chloro, bromo and iodo and preferably is either fluoro or chloro.

As used herein, the term “heteroaryl” refers to an aromatic carbocyclic group of from 1 to 15 carbon atoms and 1 to 4 heteroatoms selected from the group consisting of oxygen, nitrogen and sulfur within at least one ring (if there is more than one ring). 100801 Unless otherwise constrained by the definition for the heteroaryl substituent, such heteroaryl groups can be optionally substituted with from 1 to 5 substituents and preferably 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, substituted amino, aminoacyl, acyloxy, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and trihalomethyl. Preferred substituents include alkyl, alkoxy, halo, cyano, nitro, trihalomethyl, and thioalkoxy. Such heteroaryl groups can have a single ring (e.g., pyridyl or furyl) or multiple condensed rings (e.g., indolizinyl or benzothienyl).

“Heterocycle” or “heterocyclic” refers to a monovalent saturated or unsaturated carbocyclic group having a single ring or multiple condensed rings, from 1 to 15 carbon atoms and from 1 to 4 hetero atoms selected from the group consisting of nitrogen, sulfur and oxygen within the ring.

Unless otherwise constrained by the definition for the heterocyclic substituent, such heterocyclic groups can be optionally substituted with 1 to 5 substituents selected from the group consisting of alkyl, substituted alkyl, alkoxy, substituted alkoxy, aryl, aryloxy, halo, nitro, heteroaryl, thiol, thioalkoxy, substituted thioalkoxy, thioaryloxy, trihalomethyl, and the like. Such heterocyclic groups can have a single ring or multiple condensed rings.

As to any of the above groups that contain one or more substituents, it is understood, of course, that such groups do not contain any substitution or substitution patterns which are sterically impractical and/or synthetically non-feasible.

As used herein, “carboxylic acid derivatives” and “sulfonic acid derivatives” refer to —C(X)R³¹, —C(X)—N(R³¹)₂, —C(X)OR³¹, —C(X)SR³¹, —SO_{b}R³¹, —SO_{b}OR³¹, —SO_{b}SR³¹, or SO_{b}—N(R³¹)₂, where X is O, S or NR³¹, where R³¹ is hydrogen, alkyl, substituted alkyl or aryl, and activated derivatives thereof, such as anhydrides, esters, and halides such as chlorides, bromides and iodides.

“Pharmaceutically acceptable salts” refers to pharmaceutically acceptable salts of a compound of Formulas IIA, IIB, IIC and IID which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like can be used as the pharmaceutically acceptable salt.

### 5. BRIEF DESCRIPTION OF THE DRAWINGS

FIGS. 1A through 1H illustrate the saturation of [³H]-MRE 3008-F20 binding to A₃ adenosine receptors in human cancers. K_{D} and Bₘₐₓ values are reported in Table 8. Values are the means and S.E. of the mean of three separate experiments performed in triplicate. In the inset the Scatchard plot of the same data is shown.

FIG. 2 illustrates colony formation assay of A375 cells. Cells were treated with different drugs and colonies were counted after 7 days. The values represent the mean±SEM of four independent experiments. D=DMSO (control); T=paclitaxel, 0.75 ng/ml; M=MRE 3008F20, 10 μM; C=CI-IB-MECA, 10 μM; TM=paclitaxel plus MRE 3008F20; TC=paclitaxel plus CI-IB-MECA treated cells. *P<0.01 TM versus T. Analysis was by ANOVA followed by Dunnett's test.

FIG. 3A through 3D illustrate typical dose response curves of A375 cells exposed to increasing concentrations of cytotoxic agents vindesine (FIG. 3A, FIG. 3B) and Taxol™ brand paclitaxel (FIG. 3C, FIG. 3D). The curves with open symbols represent the cytotoxic agent alone. The curves with closed symbols represent the cytotoxic agent in the presence of 10 μM MRE 3008F2010. FIG. 3A and FIG. 3C illustrate G₂/M phases arrest calculated as percentage of untreated cells (control). FIG. 3B and FIG. 3D illustrate the accumulation (percentage of total living cells) of the sub-G₁ (apoptosis) population. Cells were fixed in 70% ethanol, stained with PI, and analysed by flow cytometry.

FIG. 4A through 4F illustrate induction of G₂/M phases arrest by MRE 3008F20 on exponentially growing A375 cells treated with paclitaxel or with vindesine. A375 cells are treated for 24 hours with drug-vehicle as control (FIG. 4A, FIG. 4D); with 1 nM vindesine (FIG. 4B), with 25 ng/ml paclitaxel (FIG. 4E); with 1 nM vindesine plus 10 μM MRE 3008F20 (FIG. 4C); or with 25 ng/ml paclitaxel plus 10 μM MRE 3008F20 (FIG. 4F). Cells were fixed in 70% ethanol, stained with PI, and analysed by flow cytometry. The percentage of cells at G₁, S and G₂/M phases was quantified. Apoptotic cells (Apo) were also detected.

FIG. 5A illustrates dose-response curve for G₂/M phases arrest of A375 cells exposed to increasing concentrations of A₃ adenosine receptor antagonists in the presence of 1 nM vindesine. Cells were fixed in 70% ethanol, stained with PI, and analysed by flow cytometry.

FIG. 5B illustrates comparison between enhancing activity to vindesine by adenosine receptor antagonists (SEC₅₀) and binding affinity to A₃ adenosine receptors (Kⱼ) in A375 cells (r=0.96; P<0.01).

FIG. 6A through 6D illustrate flow chromatogram for Rhodamine 123 (Rh 123) accumulation by A375 cells and HeL023 cells. FIG. 6A illustrates the accumulation by A375 cells. FIG. 6B illustrates the accumulation by A375 cells in the presence of 10 μM MRE 3008F20. FIG. 6C illustrates the accumulation by HeL023 cells. FIG. 6D illustrates the accumulation by HeL023 cells in the presence of 10 μM MRE 3008F20. In all cases, F_{MAX} represents the maximum load of Rh 123 (gray filled area). F_{RES} shows residual Rh 123 fluorescence after the P-gp mediated drug efflux was allowed for 3 hours (black filled area). Rh 123 unstained cell chromatogram is reported as unfilled area.

FIG. 7A illustrates the effect of inhibitors of MEK-(PD98059), ERK 1/2-(U0126) and p38^{MAPK-}(SB203580)-signalling on the G₂/M phases arrest induced by paclitaxel (filled bars) and by vindesine (empty bars). The residual G₂/M phases arrest is reported as the percentage of control cells (cells treated with paclitaxel plus MRE 3008F20 for TU, TS, TP or with vindesine plus MRE 3008F20, for VU, VS, VP.

FIG. 7B illustrates the effect of inhibitors of MEK-(PD98059), ERK 1/2-(U0126) and p38MAPK-(SB203580)-signalling on apoptosis induced by paclitaxel. T=paclitaxel (25 ng/ml), V=vindesine 1 μM, U=U0126 30 μM, P=PD98059 20 μM, S=SB203580 1 μM, M=MRE 3008F20 10 μM, C=cells treated with metabolic inhibitor vehicle (DMSO) (control). Each bar represents the mean±SE of four independent experiments performed on A375 cells. P<0.01 as follows: *TU versus internal control (paclitaxel plus MRE 3008F20); #VU versus internal control (vindesine plus MRE 3008F20); §P<0.05: 2 versus 1, 4 versus 3, 6 versus 5 and 8 versus 7. Analysis was by ANOVA followed by Dunnett's test.

FIG. 8. Apoptosis and cell cycle analysis of A375 cells cultured in normoxia or hypoxia for 24 hours. (A), Representative flow cytometric analysis of cell cycle using propidium iodide for DNA staining: shown is the pattern of A375 cells being in apoptosis and in G₀/G₁, S and G₂/M phase during normoxia and hypoxia. Apoptotic cells (Apo) with sub-diploid DNA content are reported. (B), The quantitative analysis of sub-diploid and of cells in G₀/G₁, S and G₂/M phase is given in the graph. Plots are mean±S.E. values (n=3). *P<0.0l compared with hypoxia.

FIG. 9. Induction of HIF-1 expression by hypoxia. A375 cells were cultured in normoxia for 4 hours (lane normoxia) or under hypoxic conditions for 2, 3, 4, 8, 16 and 24 hours (lanes 2-7). Whole cellular protein extracts were prepared and subjected to immunoblot assay using an anti-HIF-1α monoclonal antibody. The blot was then stripped and used to determine HIF-1β expression using an anti-HIF-1β monoclonal antibody. Tubulin shows equal loading protein.

FIG. 10. Iinduction of HIF-1α expression by adenosine. (A), A375 cells were cultured in normoxia for 4 hours (lane normoxia). A375 cells were treated without (lane 1) or with adenosine 10 nM (lane 2), 100 nM (lane 3), 1 μM (lane 4), 10 μM (lane 5) and 100 μM (lane 6) in hypoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody. Tubulin shows equal loading protein. (B), Typical dose response curve of A375 cells exposed to adenosine in hypoxia is shown. The HIF-1α immunoblot signals were quantified using molecular analyst/PC densitometry software (Bio-Rad). The mean densitometry data from 12 independent experiments (one of which is shown in Panel A) were normalized to the result obtained in cells in the absence of adenosine (control). Plots are mean±S.E. values (n=12). (C), Effect of A₁, A_{2A}, A_{2B} and A₃ adenosine receptor antagonists. A375 cells were treated without (lane 1, control) or with adenosine 100 μM (lanes 2-6) and exposed to the A₁ receptor antagonist DPCPX 100 nM (lane 3), or A_{2A} receptor antagonist SCH 58261 100 nM (lane 4), or A_{2B} receptor antagonist MRE 2029F20 100 nM (lane 5), or A₃ receptor antagonist MRE 3008F20 100 nM (lane 6) in hypoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody. Tubulin shows equal loading protein. (D), The immunoblot signals were quantified using molecular analyst/PC densitometry software (Bio-Rad). The mean densitometry data from 5 independent experiments (one of which is shown in Panel C) were normalized to the result obtained in cells in the absence of adenosine (control). Plots are mean±S.E. values (n=5). *P<0.01 compared with the control.

FIG. 11. Induction of HIF-1 expression by A₃ receptor stimulation: time course. (A), A375 cells were cultured in normoxia for 4 hours (lane normoxia) or under hypoxic conditions in the absence (−) or in the presence (+) of the A₃ receptor agonist Cl-IB-MECA (100 nM) for 2, 4, 8, 16 and 24 hours. Whole cellular protein extracts were prepared and subjected to immunoblot assay using an anti-HIF-1α monoclonal antibody. The blot was then stripped and used to determine HIF-1β expression using an anti-HIF-1β monoclonal antibody. Tubulin shows equal loading protein. (B), The immunoblot signals were quantified using molecular analyst/PC densitometry software (Bio-Rad). The mean densitometry data from 3 independent experiments (one of which is shown in Panel A) were normalized to the result obtained in cells in the absence of Cl-IB-MECA after 4 hours of hypoxia (control). Plots are mean±S.E. values (n=3). *P<0.01 compared with the control.

FIG. 12. Induction of HIF-1α expression by A₃ receptor stimulation: dose response. (A), A375 cells were treated without (lane 1) or with Cl-IB-MECA 0.1 nM (lane 2), 1 nM (lane 3), 10 nM (lane 4) 100 nM (lane 5) and 1 μM (lane 6) in normoxia and in hypoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody. (B), Typical dose response curve of A375 cells exposed to adenosine in hypoxia is shown. The immunoblot signals were quantified using molecular analyst/PC densitometry software (Bio-Rad). The mean densitometry data from 12 independent experiments (one of which is shown in Panel A) were normalized to the result obtained in cells in the absence of Cl-IB-MECA (control). Plots are mean±S.E. values (n=12).

FIG. 13. Effect of A₃ receptor antagonist MRE 3008F20. (A), A375 cells were treated in hypoxia for 4 hours without (−) or with (+) Cl-IB-MECA 10 nM, MRE 3008F20 10 nM (lanes 3, 4) and MRE 3008F20 100 nM (lanes 5, 6). Cellular extracts were prepared and subjected to immunoblot assay using an anti-HIF-1α monoclonal antibody. The blot was then stripped and used to determine HIF-1β expression using an anti-HIF-1β monoclonal antibody. (B), The immunoblot signals were quantified using molecular analyst/PC densitometry software (Bio-Rad). The mean densitometry data from independent experiments (one of which is shown in Panel A) were normalized to the result obtained in hypoxic cells in the absence of Cl-IB-MECA (control, lane 1). Plots are mean±S.E. values (n=3); *P<0.01 compared with the control. (C), A375 cells were treated in hypoxia for 4 hours without (lane 1) or with Cl-IB-MECA 10 nM (lanes 2-6) and MRE 3008F20 0.3 nM (lane 3), 1 nM (lane 4), 3 nM (lane 5) and 10 nM (lane 6). Cellular extracts were prepared and subjected to immunoblot assay using an anti-HIF-1α monoclonal antibody. The blot was then stripped and used to determine HIF-1β expression using an anti-HIF-1β monoclonal antibody. (D), Typical dose response curve of A375 cells exposed to MRE 3008F20 in hypoxia is shown. The immunoblot signals were quantified using molecular analyst/PC densitometry software (Bio-Rad). The mean densitometry data from 3 independent experiments (one of which is shown in Panel C) were normalized to the result obtained in cells in the absence of Cl-IB-MECA (control). Plots are mean±S.E. values (n=3).

FIG. 14. A₃ receptor expression silencing by siRNA transfection. (A), Analysis of siRNA transfection efficiency in A375 cells. Representative flow chromatograms of siRNA-FITC accumulation (gray filled area) in A375 cells transfected with siRNA-FITC. Unfilled area shows A375 cells transfected with RNAiFect™ Transfection reagent without siRNA_{A3}. Fluorescence was quantified by flow cytometry 5 hours post-transfection. (B), Relative A₃ receptor mRNA quantification, related to β-actin mRNA, by Real-Time RT-PCR. A375 cells were transfected by RNAiFect™ Transfection reagent or siRNA_{A3} and cultured for 24, 48 and 72 hours. Plots are mean±S.E. values (n=3); *P<0.01 compared with the control. (C), Western blot analysis using an anti-A₃ receptor polyclonal antibody of protein extracts from A375 cells treated by RNAiFect™ Transfection reagent (control) or siRNA_{A3} and cultured for 24, 48 and 72 hours in normoxia. Tubulin shows equal loading protein. (D), Densitometric quantification of A₃ receptor Western blot; plots are mean±S.E. values (n=5); *P<0.01 compared with the control. (E), Western blot analysis using an anti-HIF-1α monoclonal antibody of protein extracts from A375 cells treated by control-siRNA (−) or siRNA_{A3} (+) for 72 hours and cultured with (+) or without (−) Cl-IB-MECA 100 nM for 4 hours in hypoxia. Tubulin shows equal loading protein.

FIG. 15. A₃ receptor stimulation induces HIF-1α accumulation in various human cell lines under hypoxia. NCTC 2544 keratinocytes, U87MG glioblastoma, U2OS osteosarcoma human cells were treated without (−) or with (+) Cl-IB-MECA 100 nM in hypoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody.

FIG. 16. A₃ receptor stimulation induces HIF-1α accumulation through a transcription-independent pathway. (A), A375 cells were pretreated with actinomycin D (10 μg/ml) for 30 minutes and then exposed to hypoxia. HIF-1α accumulation was induced by the exposure of A375 cells to Cl-IB-MECA 100 nM (+) for 4 hours in hypoxia in the absence (lane 2) or in the presence (lane 4) of actinomycin D. Cellular extracts were prepared and subjected to immunoblot assay using an anti-HIF-1α monoclonal antibody. Tubulin shows equal loading protein. (B), The immunoblot signals were quantified using molecular analyst/PC densitometry software (Bio-Rad). The mean densitometry data from independent experiments (one of which is shown in Panel A) were normalized to the result obtained in cells in the absence of Cl-IB-MECA after 4 hours of hypoxia (control=lane 1). Plots are mean±S.E. values (n=3); *P<0.01 compared with the control.

FIG. 17. Induction of HIF-1α accumulation by A₃ receptor stimulation in normoxia. (A), A375 cells were exposed to 100 μM CoCl₂ alone (lane 1) or in the presence of Cl-IB-MECA 1 nM (lane 2), 10 nM (lane 3), 100 nM (lane 4), 1 μM (lane 5) and 10 μM (lane 6) in normoxia for 4 hours. Cellular extracts were prepared and subjected to immunoblot assay using an anti-HIF-1α monoclonal antibody. The blot was then stripped and used to determine HIF-1β expression using an anti-HIF-1β monoclonal antibody. (B), A375 cells were exposed to 100 μM CoCl₂ for 4 hours in normoxia. HIF-1α accumulation was induced by the exposure of A375 cells to Cl-IB-MECA 100 nM (+) for 4 (lanes 1 and 4) or 6 hours (lanes 5 and 6) in the absence (lanes 1 and 2) or in the presence (lanes 3-6) of cycloheximide (1 μM). Cellular extracts were prepared and subjected to immunoblot assay using an anti-HIF-1α monoclonal antibody. Tubulin shows equal loading protein.

FIG. 18. A₃ receptor activation does not affect HIF-1α degradation in normoxia. (A), A375 cells were incubated in hypoxia in the absence (lanes 1 to 4) or in the presence of Cl-IB-MECA 100 nM (lanes 5 to 8). After 4 hours, melanoma cells were exposed to normoxia and a time-course of HIF-1α disappearance was performed at 0, 5, 10 and 15 minutes. Cellular extracts were prepared and subjected to immunoblot assay using an anti-HIF-1α monoclonal antibody. Tubulin shows equal loading protein. (B), The immunoblot signals were quantified using molecular analyst/PC densitometry software (Bio-Rad). The mean densitometry data from 3 independent experiments (one of which is shown in Panel A) were normalized to the result obtained in cells at time 0 (control). The fraction of HIF-1α remaining is indicated.

FIG. 19. Role of p38, p44 and p42 MAPKs in A₃ signalling. (A), A375 cells were pretreated with or without SB202190 (1 and 10 μM) or U0126 (10 and 30 μM) and then exposed to Cl-IB-MECA 100 nM (+) or to drug vehicle (−) for 4 hours in hypoxia. Cellular extracts were prepared and subjected to immunoblot assay using an anti-HIF-1α monoclonal antibody. Tubulin shows equal loading protein. (B), A₃ stimulation via Cl-IB-MECA induces p44/p42 activation after 4 hours hypoxia in A375 cells. Cl-IB-MECA 0 (lane C), 10 (lane 1), 100 (lane 2), 500 (lane 3) and 1000 (lane 4) nM was added to A375 cells. After 4 hours cells were harvested and subjected to immunoblot assay using antibodies specific for phosphorylated (Thr183/Tyr185) or total p44/p42 MAPKs. (C), The immunoblot signals were quantified using molecular analyst/PC densitometry software (Bio-Rad). Densitometric analysis of p44 and p42 phosphorylated isoforms is reported. The mean densitometry data from 3 independent experiments (one of which is shown in Panel B) were normalized to the results obtained in cells in the absence of Cl-IB-MECA (lane C). Plots are mean±S.E. values (n=3); *P<0.01 compared with the control (lane C). (D), A₃ stimulation via Cl-IB-MECA induces p38 activation after 4 hours hypoxia in A375 cells. Cl-IB-MECA 0 (lane C), 10 (lane 1), 100 (lane 2), 500 (lane 3) and 1000 (lane 4) nM was added to A375 cells. After 4 hours cells were harvested and subjected to immunoblot assay using antibodies specific for phosphorylated (Thr180/Tyr182) or total p38 MAPKs. (E), The immunoblot signals were quantified using molecular analyst/PC densitometry software (Bio-Rad). Densitometric analysis of p38 phosphorylated isoforms is reported. The mean densitometry data from 3 independent experiments (one of which is shown in Panel D) were normalized to the result obtained in cells in the absence of Cl-IB-MECA (lane C). Plots are mean±S.E. values (n=3); *P<0.01 compared with the control (lane C).

### 6. DETAILED DESCRIPTION OF THE INVENTION

The present invention is based, in part, on the surprising discovery by the inventors that adenosine, a purine nucleoside present within hypoxic regions of solid tumors, modulates hypoxia-inducible factor 1 (HIF-1) expression. HIF-1, a heterodimeric transcription factor composed of HIF-1α and HIF-1β subunits, is involved in tumor growth and angiogenesis. The inventors have found that in the human A375 melanoma cell line adenosine up-regulates HIF-1α protein expression in response to hypoxia in a dose- and time-dependent manner. The response to adenosine was not blocked by A₁, A_{2A} or A_{2B} receptor antagonists, while it was abolished by A₃ receptor antagonists. The inventors have found that Cl-IB-MECA, an adenosine analogue binding with high affinity to A₃ receptors, mimicked adenosine effect in hypoxic cells. Furthermore, A₃ receptor antagonists prevented HIF-1α protein accumulation in response to A₃ receptor stimulation. Although not intending to be bound by a particular mechanism of action, the response to adenosine is generated at the cell surface since the inhibition of A₃ receptor expression, by using small interfering RNA, abolished the nucleoside effects. The main intracellular signaling pathways sustained by A₃ receptor stimulation in hypoxia involve p44/p42 mitogen-activated protein kinase (MAPK) and p38 MAPK activation. The inventors have thus found for the first time that adenosine plays a critical role in HIF-1α regulation in hypoxia.

The inventors have found that compounds which are antagonists of the adenosine receptors, preferably the A₃ receptor, inhibit the protective effect of adenosine on growing tumor cells when such cells are starved of oxygen (i.e., before an adequate vasculature is developed, or when anti-angiogenesis agents are administered. Although not intending to be bound by a particular mechanism of action, the antagonists of the invention can bind in the same site as adenosine, or can be allosteric antagonists (i.e., bind at a site different from where adenosine binds, but adversely affect the ability of adenosine to bind to the site or adversely affect the ability of adenosine, once bound to the adenosine receptors particularly A₃ receptors, to protect growing tumor cells).

Accordingly, the present invention relates to methods for the treatment, prevention, and/or management of diseases or disorders associated with overexpression of HIF-1α and/or increased HIF-1α activity (e.g., cancer, respiratory disorders such as asthma and obstructive pulmonary disorders) by using adenosine receptor antagonists, particularly A₃ receptor antagonists, alone or in combination with A₁ receptor antagonists. Without being bound to a particular mechanism of action, administration of antagonists for the adenosine receptors antagonizes the protective effects against hypoxia and renders those cells susceptible to destruction due to hypoxia. Since the adenosine receptors, in particular, the A₃ receptors, are responsible for sustained cellular protection against ischemia, antagonists for the adenosine receptors, particularly A₃ receptors are particularly effective in enhancing the activity of anti-tumor agents.

The methods and compositions of the invention comprising A₃ receptor antagonists are particularly useful when the levels of HIF-1α expression and/or activity are elevated above the standard or background level, as determined using methods known to those skilled in the art and dislosed herein. As used herein, “elevation” of a measured level of HIF-1α relative to a standard level means that the amount or concentration of HIF-1α in a sample or subject is sufficiently greater in a subject or sample relative to the standard as detected by any method now known in the art or to be developed in the future for measuring HIF-1α levels. For example, elevation of the measured level relative to a standard level may be any statistically significant elevation detectable. Such an elevation in HIF-1α expression and/or activity may include, but is not limited to about a 10%, about a 20%, about a 40%, about an 80%, about a 2-fold, about a 4-fold, about an 10-fold, about a 20-fold, about a 50-fold, about a 100-fold, about a 2 to 20 fold, 2 to 50 fold, 2 to 100 fold, 20 to 50 fold, 20 to 100 fold, elevation, relative to the standard. The term “about” as used herein, refers to levels of elevation of the standard numerical value plus or minus 10% of the numerical value.

The term “standard level” or “background level” as used herein refers to a baseline amount of HIF-1α level as determined in one or more normal subjects, i.e., a subject with no known history of past or current diseases, disorders or cancer. For example, a baseline may be obtained from at least one subject and preferably is obtained from an average of subjects (e.g., n=2 to 100 or more), wherein the subject or subjects have no prior history of diseases, disorders or cancer, especially no prior history of diseases associated with an aberrant level of expression and/or activity of HIF-1α.

In the present invention, the measurement of HIF-1α level may be carried out using an HIF-1α probe or a HIF-1α activity assay (see Section 5.4.4). As used herein, reference to measuring a level of HIF-1α in a method of the invention relates to any proxy for HIF-1α levels. For example, such levels may include, but are not limited to, the abundance of HIF-1α nucleic acid or amino acid sequences in a sample from a subject. A level of HIF-1α may correspond to the abundance of full-length HIF-1α protein. Alternatively, a level of HIF-1α, may correspond to abundance of a fragment, analog or derivative of HIF-1α protein. A level of HIF-1α can be determined by measuring the abundance of nucleic acids (or sequences complementary thereto) that corresponds to all or fragments of HIF-1α. In a preferred embodiment, the abundance of mRNA encoding HIF-1α is measured.

As used herein, a probe with which the amount or concentration of HIF-1α can be determined, includes but is not limited to an antibody, an antigen, a nucleic acid, a protein, or a small molecule. In a specific embodiment, the probe is the HIF-1α protein or a fragment thereof. In another embodiment, the probe is an antibody that immunospecifically binds to HIF-1α, such as e.g., a monoclonal antibody or a binding fragment thereof.

In a specific embodiment, measuring a level of HIF-1α comprises testing at least one aliquot of the sample, said step of testing comprising: (a) contacting the aliquot with an antibody or a fragment thereof that is immunospecific for HIF-1α, and (b) detecting whether and how much binding has occurred between the antibody or a fragment thereof and at least one species of HIF-1α in the aliquot. In yet another specific embodiment, measuring a level of HIF-1α comprises testing at least one aliquot, said step of testing comprising: (a) contacting the aliquot with a nucleic acid probe that is hybridizable to HIF-1α mRNA, and (b) detecting whether and how much hybridization has occurred between the nucleic acid probe and at least one species of HIF-1α mRNA in the aliquot. In both embodiments measuring a level of HIF-1α involves quantitating the amount of complex formation. For example the amount of complex formation between the antibody or a fragment thereof and at least one species of HIF-1α in the aliquot would correlate with the amount of at least one species of HIF-1α in the aliquot of the sample.

In a further specific embodiment, the antibody or other probe is labeled with a detectable marker. In yet another specific embodiment, the detectable marker is a chemiluminescent, enzymatic, fluorescent, or radioactive label.

The therapeutic methods of the invention comprising administering a therapeutically effective amount of an A₃ receptor antagonist of the invention improve the therapeutic efficacy of treatment for diseases or disorders associated with overexpression of HIF-1α and/or increased HIF-1α activity (e.g., cancer, respiratory disorders such as asthma and obstructive pulmonary disorders) relative to the traditional modes of such therapies. Preferably the methods of the invention reduce the HIF-1α level to the background level within within one day, one week, 1 month, or 2 months of the commencement of the therapeutic regime. Preferably, once the methods of the invention reduce the level of HIF-1α to a particular level, that level is maintained during the treatment regimen, such that the maintained level of HIF-1α is sufficient and effective to result in regression of the disease, e.g., cancer.

In a most preferred embodiment, the methods of the invention result in a reduction of HIF-1α level to the background level. The invention encompasses reduction of the HIF-1α level to a level which is within about 10%, about 20%, about 30%, about 40%, about 50% of the background level.

In a preferred specific embodiment, the invention encompasses a method for treatment, prevention and/or management of diseases or disorders associated with overexpression of HIF-1α and/or increased HIF-1α activity (e.g., cancer, respiratory disorders such as asthma and obstructive pulmonary disorders) comprising administering a therapeutically and/or prophylactically effective amount of an A₃ receptor antagonist compound as disclosed herein.

The invention also encompasses a method for determining the prognosis of a a disease or disorder associated with overexpression of HIF-1α and/or increased HIF-1α activity in a subject. Preferably, the subject is human. In some embodiments, the subject has been previously treated with a therapy regimen. The invention encompasses measuring at least a level of HIF-1α in a subject to determine if the subject is in need of the therapeutic and or prophylcatic methods of the inventon. The invention encompasses measuring a level of at least one HIF-1α in a sample obtained from the subject and comparing the level measured to a standard level, wherein elevation of the measured level of at least one HIF-1α relative to the standard level indicates that the subject is at an increased risk for progression of the disease or disorder, e.g., metastasis of the cancer.

The invention encompasses compounds which are adenosine receptor antagonists particularly A₃ antagonists for use in the methods of the invention. Examples of such compounds are disclosed in U.S. Pat. Nos. 6,326,390; 6,407,236; 6,448,253; 6,358,964; and U.S. Publication Nos. 2003/0144266 and 2004/0067932; all of which are incorporated herein by reference in their entireties. In addition, other suitable adenosine A₃ antagonists are disclosed in the references listed in Table 1, supra, the disclosures of which are hereby incorporated by reference in their entireties.

The A₃ receptor antagonists of the invention are particularly useful for prevention, treatment, and/or management of cancer, for example, as a single agent therapy or in combination with other modes of therapy for cancer. In preferred embodiments, the invention encompasses methods of treatment, prevention, or management of cancers which are A₃ expressing cancers including, but not limited to, human leukemia, melanoma, pancreatic carcinoma, ovarian carcinoma, breast carcinoma, prostrate carcinoma, colon carcinoma, lung carcinoma, malignant melanomas, histiocytic lymphoma, and some forms of astrocytoma cells. In other preferred embodiments, the invention encompasses methods of treatment, prevention, or management which have high level of expression of HIF-1α including, but not limited to, cervical cancer (early stage), lung cancer (non-small cell lung carcinoma), breast cancer (including lymph node positive breast cancer and lymph node negative breast cancer), oligodendroglioma, orpharyngeal squamous cell carcinoma, ovarian cancer, oesophageal cancer, endometrial cancer, head and neck cancer, gastrointestinal stromal tumor of the stomach The methods of the invention are particularly useful in cancer therapy for providing tissue selectivity, such that the biological effect is observed in tumoral hypoxic cells, where high adenosine concentrations has resulted in increased HIF-1α accumulation.

The methods and compositions of the invention comprising A₃ receptor antagonists (or A₁ receptor antagonists or a combination thereof) are particularly useful for the treatment, inhibition or regression of solid tumors. As used herein, “solid tumors” refer to a locus of tumor cells where the majority of the cells are tumor cells or tumor-associated cells, including but not limited to laryngeal tumors, brain tumors, and other tumors of the head and neck; colon, rectal and prostate tumors; breast and thoracic solid tumors; ovarian and uterine tumors; tumors of the esophagus, stomach, pancreas and liver; bladder and gall bladder tumors; testicular cancer; skin tumors such as melanomas. Moreover, the tumors encompassed within the invention can be either primary or a secondary tumor resulting from metastasis of cancer cells elsewhere in the body to the chest.

The invention encompasses use of the adenosine receptor antagonists of the invention, particulalry A₃ receptor antagonists in inhibiting tumor growth in a mammal, including humans. The invention encompasses methods for administering an effective amount of an adenosine receptor antagonists, preferably A₃ receptor antagonists to inhibit the ability of adenosine to protect tumor cells.

In other embodiments, the present invention encompasses methods for treatment, prevention and/or management of cancer comprising administering an adenosine receptor antagonist, particularly an A₃ receptor antagonist combination with other therapeutic and/or prophylactic agents, e.g., cytotoxic agents. The inventors have surprisingly found that adenosine A₃ receptor antagonists synergistically enhance cytotoxic treatment and counter some forms of multi-drug resistance. Although not intending to be bound by a particular mechanism of action, the combination therapies of the invention will attack the tumor cell directly, inhibit growth of new blood vessels around the tumor cell, and, by virtue of the adenosine A₃ antagonists, inhibit the ability of the cell to survive without the growth of new blood vessels. Inventors have discovered that high affinity adenosine A₃ receptor antagonists are useful as enhancers for many chemotherapeutic treatments of adenosine A₃ receptor expressing cancers. Surprisingly, high affinity adenosine A₃ receptor antagonists also counter P-glycoprotein (P-gp) effuse pump multi-drug resistance (MDR). Finally, high affinity adenosine A₃ receptor antagonists are helpful in reducing or ameliorating side effects of cytotoxic agents, e.g., taxane

In other embodiments, the present invention encompasses the use of high affinity adenosine A₃ receptor antagonists for enhancing chemotherapeutic treatment of cancers expressing adenosine A₃ receptors and countering multi-drug resistance in cancers expressing P-glycoprotein or MRP. In preferred embodiments, adenosine A₃ receptor antagonists are administered before or during administration of a taxane family, vinca alkaloid, camptothecin or antibiotic compound. Preferred high affinity A₃ receptor antagonists include compounds of the general formulas IIA, IIB, IIC and IID described herein, vide infra.

The present invention encompasses therapies which involve administering one or more of the compounds of the invention, to an animal, preferably a mammal, and most preferably a human, for preventing, treating, or ameliorating symptoms associated with a cancer, a disease or disorder associated with hypoxia-inducible factor 1-α (HIF-1α).

The invention further provides a pharmaceutical composition comprising a therapeutically or prophylactically effective amount of a compound of the invention that specifically binds an A₁ or A₃ receptor and a pharmaceutically acceptable carrier. Preferably the pharmaceutical formulation includes an A₃ receptor antagonist and one or more excipients. The formulations can also include other anti-tumor agents, including cytotoxic agents and other anti-angiogenesis agents, including adenosine A_{2A} antagonists, such as ZM241385 and SCH 5861, adenosine A_{2B} antagonists, such as MRE-20290-F20 and AS-16, and anti-VEGF antibodies, including humanized and chimeric antibodies. In a preferred embodiment, the composition includes an effective amount to inhibit tumor growth of an adenosine A₃ receptor antagonist, a cytotoxic agent, and an anti-angiogenesis agent.

6.1 Compounds of the Invention

6.1.1 Compounds of the General Formula IIA, IIB and IIC

In a specific embodiment, the invention provides methods for treating a disease or disorder associated with HIF-1α in a patient, comprising administering to a patient in need thereof an effective amount of a compound or a pharmaceutically acceptable salt of the compound having the following general formulas IIA and IIB:

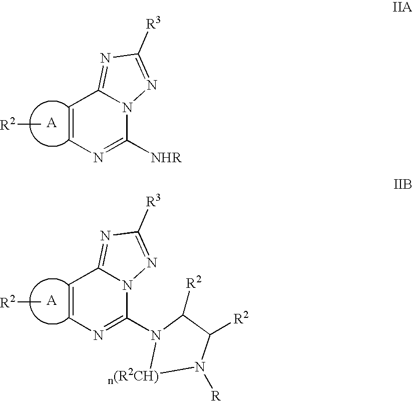

wherein:

A is imidazole, pyrazole, or triazole;

R is —C(X)R¹, —C(X)—N(R¹)₂, —C(X)OR¹, —C(X)SR¹, —SO_{b}R¹, —SO_{b}OR¹, —SO_{b}SR¹, or SO_{b}—N(R¹)₂;

R¹ is hydrogen, alkyl, substituted alkyl, alkenyl, substituted alkenyl, alkynyl, substituted alkynyl, aryl, substituted aryl, heteroaryl, substituted heteroaryl, heterocyclic, substituted heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms such as N, O, S;

R² is hydrogen, alkyl, alkenyl, alkynyl, substituted alkyl, substituted alkenyl, substituted alkynyl, aralkyl, substituted aralkyl, aryl, substituted aryl, heteroaryl, or substituted heteroaryl;

R³ is furan, pyrrole, thiophene, benzofuran, indole, benzothiophene, optionally substituted with one or more substituents as described herein for substituted heteroaryl rings;

X is O, S, or NR¹;

b is 1 or 2.

Preferably, R¹ is hydrogen; C₁ to C₈ alkyl; C₂ to C₇ alkenyl, C₂ to C₇ alkynyl; C₃ to C₇ cycloalkyl; C₁ to C₅ alkyl substituted with one or more halogen atoms, hydroxy groups, C₁ to C₄ alkoxy, C₃ to C₇ cycloalkyl or groups of formula —NR³²R³², —C(O)NR³²R³²; aryl, substituted aryl wherein the substitution is selected from the group consisting of C₁ to C₄ alkoxy, C₁ to C₄ alkyl, nitro, amino, cyano, C₁ to C₄ haloalkyl, C₁ to C₄ haloalkoxy, carboxy, carboxyamido; C₇ to C₁₀ aralkyl in which the aryl moiety can be substituted with one or more of the substituents indicated above for the aryl group; a group of formula —(CH₂)ₘ-Het, wherein Het is a 5-6 membered aromatic or non aromatic heterocyclic ring containing one or more heteroatoms selected from the group consisting of N, O, and S and m is an integer from 0 to 5, and in some embodiments from 1-5.

Preferred C₁ to C₈ alkyl groups are methyl, ethyl, propyl, butyl and isopentyl. Examples of C₃ to C₇ cycloalkyl groups include cyclopropyl, cyclopentyl, and cyclohexyl. Examples of C₁ to C₅ alkyl groups substituted with C₃ to C₇ cycloalkyl groups include cyclohexylmethyl, cyclopentylmethyl, and 2-cyclopentylethyl. Examples of substituted C₁ to C₅ alkyl groups include 2-hydroxyethyl, 2-methoxyethyl, trifluoromethyl, 2-fluoroethyl, 2-chloroethyl, 3-aminopropyl, 2-(4-methyl-1-piperazine)ethyl, 2-(4-morpholinyl)ethyl, 2-aminocarbonylethyl, 2-dimethylaminoethyl, 3-dimethylaminopropyl. Aryl is preferably phenyl, optionally substituted with Br, Cl, F, methoxy, nitro, cyano, methyl, trifluoromethyl, difluoromethoxy groups. Examples of 5 to 6 membered ring heterocyclic groups containing N, O and/or S include piperazinyl, morpholinyl, thiazolyl, pyrazolyl, pyridyl, furyl, thienyl, pyrrolyl, triazolyl, tetrazolyl. Examples of C₇ to C₁₀ aralkyl groups comprise benzyl or phenethyl optionally substituted by one or more substituents selected from Cl, F, methoxy, nitro, cyano, methyl, trifluoromethyl, and difluoromethoxy.

Preferably, R¹ is hydrogen, C₁ to C₈ alkyl, aryl or C₇ to C₁₀ aralkyl, optionally substituted, preferably with halogen atoms. Preferably, X is O, R² is C₂-C₃ alkyl or substituted alkyl and R³ is furan.

In a specific embodiment R² is alkyl.

Particularly preferred compounds are those in which R is a phenethyl group in which the phenyl ring is substituted with one or more substituents selected from the group consisting of chlorine, fluorine atoms, methoxy, nitro, cyano, methyl, trifluoromethyl, and difluoromethoxy groups.

The possible meanings of A can be represented by the following structural formulae:

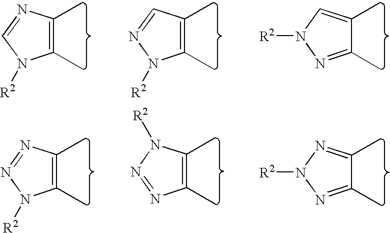

In a particular embodiment, the invention provides methods for treating a disease or disorder associated with HIF-1α in a patient, comprising administering to a patient in need thereof an effective amount of a compound or a pharmaceutically acceptable salt of the compound having the general formula IIA:

wherein:

A is imidazole, pyrazole, or triazole;

R is —C(X)R¹, —C(X)—N(R¹)₂, —C(X)OR¹, —C(X)SR¹, —SOₙR—, —SOₙSR¹, or —SOₙN(R¹)₂;

R¹ is hydrogen, alkyl, substituted alkyl, alkenyl, substituted alkenyl, alkynyl, substituted alkynyl, aryl, substituted aryl, heteroaryl, heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms;

R² is hydrogen, alkyl substituted alkyl alkenyl, aralkyl, substituted aralkyl, heteroaryl, substituted heteroaryl or aryl;

R³ is furan, pyrrole, thiophene, benzofuran, benzypyrrole, benzothiophene, optionally substituted with one or more substituents selected from the group consisting of hydroxy, acyl, alkyl, alkoxy, alkenyl, alkynyl, substituted alkyl, substituted alkoxy, substituted alkenyl, substituted alkynyl, amino, substituted amino, aminoacyl, acyloxy, acylamino, alkaryl, aryl, substituted aryl, aryloxy, azido, carboxyl, carboxylalkyl, cyano, halo, nitro, heteroaryl, heteroaryloxy, heterocyclic, heterocyclooxy, aminoacyloxy, thioalkoxy, substituted thioalkoxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and trihalomethyl;

X is O, S, or NR¹; and

n is 1 or 2.

In a specific embodiment of the invention the method is conducted using the compound of the general formula IIA, wherein R² is selected from the group consisting of hydrogen, alkyl, alkenyl and aryl.

In another specific embodiment of the invention the method is conducted using the compound of the general formula IIA, wherein A is a triazolo ring.

In another specific embodiment of the invention the method is conducted using the compound of the general formula IIA, wherein A is a pyrazolo ring.

In a specific embodiment, the method uses as high affinity adenosine A3 receptor antagonist the “phenyl-carbamoyl-amino” compounds of the general formula IIC and pharmaceutical salts thereof:

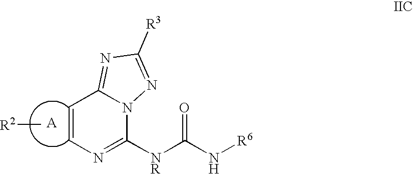

wherein:

A is imidazole, pyrazole, or triazole;

R² is hydrogen, alkyl, substituted alkyl, alkenyl, aralkyl, substituted aralkyl, heteroaryl, substituted heteroaryl or aryl;

R³ is furan; and

R⁶ is aryl, substituted aryl, heteroaryl, substituted heteroaryl, heterocycle or substituted heterocycle.

Non-limiting examples of the compounds of the general formula IIA and IIC include compounds listed below in Tables 2 and 3.

TABLE 2

| # | Compound |
|---|---|
| 1c | 5-[[(3-Chlorophenyl)amino]carbonyl]amino-8-methyl-2-(2-furyl)-pyrazolo[4,3-e]- |
| | 1,2,4-triazolo[1,5-c]pyrimidine, |
| 2c | 5-[[(4-Methoxyphenyl)amino]carbonyl]amino-8-methyl-2-(2-furyl)-pyrazolo[4,3- |
| | e]-1,2,4-triazolo[1,5-c]pyrimidine, |
| 3c | 5-[[(3-Chlorophenyl)amino]carbonyl]amino-8-ethyl-2-(2-furyl)-pyrazolo[4,3-e]- |
| | 1,2,4-triazolo[1,5-c]pyrimidine, |
| 4c | 5-[[(4-Methoxyphenyl)amino]carbonyl]amino-8-ethyl-2-(2-furyl)-pyrazolo[4,3-e]- |
| | 1,2,4-triazolo[1,5-c]pyrimidine, |
| 5c | 5-[[(3-Chlorophenyl)amino]carbonyl]amino-8-propyl-2-(2-furyl)-pyrazolo[4,3-e]- |
| | 1,2,4-triazolo[1,5-c]pyrimidine, |
| 6c | 5-[[(4-Methoxyphenyl)amino]carbonyl]amino-8-propyl-2-(2-furyl)-pyrazolo[4,3-e]- |
| | 1,2,4-triazolo[1,5-c]pyrimidine, |
| 7c | 5-[[(3-Chlorophenyl)amino]carbonyl]amino-8-butyl-2-(2-furyl)-pyrazolo[4,3-e]- |
| | 1,2,4-triazolo[1,5-c]pyrimidine, |
| 8c | 5-[[(4-Methoxyphenyl)amino]carbonyl]amino-8-butyl-2-(2-furyl)-pyrazolo[4,3-e]- |
| | 1,2,4-triazolo[1,5-c]pyrimidine, |
| 9c | 5-[[(3-Chlorophenyl)amino]carbonyl]amino-8-isopentyl-2-(2-furyl)-pyrazolo[4,3- |
| | e]-1,2,4-triazolo[1,5-c]pyrimidine, |
| 10c | 5-[[(4-Methoxyphenyl)amino]carbonyl]amino-8-isopentyl-2-(2-furyl)-pyrazolo[4,3- |
| | e]-1,2,4-triazolo[1,5-c]pyrimidine, |
| 11c | 5-[[(3-Chlorophenyl)amino]]carbonyl]amino-8-(2-isopentenyl)-2-(2- |
| | furyl)pyrazolo[4,3-e]1,2,4-triazolo[1,5-c]pyrimidine, |
| 12c | 5-[[(4-Methoxyphenyl)amino]carbonyl]amino-8-(2-isopentenyl)-2-(2-furyl)- |
| | pyrazolo[4,3-e]-1,2,4-triazolo[1,5-c]pyrimidine, |
| 13c | 5-[[(3-Chlorophenyl)amino]carbonyl]amino-8-(2-phenylethyl)-2 (2-furyl)- |
| | pyrazolo[4,3-e]-1,2,4-triazolo[1,5-c]pyrimidine, |
| 14c | 5-[[(4-Methoxyphenyl)amino]carbonylamino-8-(2-phenylethyl)-2-(2-furyl)- |
| | pyrazolo[4,3-e]-1,2,4-triazolo[1,5-c]pyrimidine, |
| 15c | 5-[[(3-Chlorophenyl)amino]carbonyl]amino-8-(3-phenylpropyl)-2-(2-furyl)- |
| | pyrazolo[4,3-e]-1,2,4-triazolo[1,5-c]pyrimidine, |
| 16c | 5-[[(4-Methoxyphenyl)amino]carbonyl]amino-8-(3-phenylpropyl)-2-(2-furyl)- |
| | pyrazolo[4,3-e]-1,2,4-triazolo[1,5-c]pyrimidine, |
| 17c | 5-[(Benzyl)carbonyl]amino-8-isopentyl-2-(2-furyl)-pyrazolo[4,3-e]-1,2,4- |
| | triazolo[1,5-c]pyrimidine, |
| 18c | 5-[(Benzyl)carbonyl]amino-8-(3-phenylpropyl)-2-(2-furyl)-pyrazolo[4,3-e]-1,2,4- |
| | triazolo[1,5-c]pyrimidine. |
| 101d | N-[4-(diethylamino)phenyl]-N′-[2-(2-furyl)-8-methyl-8H-pyrazolo[4,3- |
| | e][1,2,4]triazolo[1,5-c]pyrimidin-5-yl]urea |
| 102d | N-[8-methyl-2-(2-furyl)-8H-pyrazolo[4,3-e][1,2,4]triazolo[1,5-c]pyrimidin-5-yl]-N′- |
| | [4-(diethylamino)phenyl]urea hydrochloride |
| 103d | N-[8-methyl-2-(2-furyl)-8H-pyrazolo[4,3-e][1,2,4]triazolo[1,5-c]pyrimidin-5-yl]-N′- |
| | [4-(dimethylamino)phenyl]urea hydrochloride |
| 104d | N-(2-(2-furyl)-8-methyl-8H-pyrazolo[4,3-e][1,2,4]triazolo[1,5-c] pyrimidin-5-yl)- |
| | N′-[4-(morpholin-4-ylsulfonyl)phenyl]urea |
| 105d | N-[2-(2-furyl)-8-methyl-8H-pyrazolo[4,3-e][1,2,4]triazolo[1,5-c]pyrimidin-5-yl]- |
| | N′-{4-[(4-methylpiperazin-1-yl)sulfonyl]-phenyl}urea |
| 106d | N-[2-(2-furyl)-8-methyl-8H-pyrazolo[4,3-e][1,2,4]triazolo[1,5-c]pyrimidin-5-yl]-N′- |
| | pyridin-4-yl urea |
| 107d | N-[2-(2-furyl)-8-methyl-8H-pyrazolo[4,3-e][1,2,4]triazolo[1,5-c]pyrimidin-5-yl]-N′- |
| | pyridin-4-ylurea hydrochloride |

TABLE 3

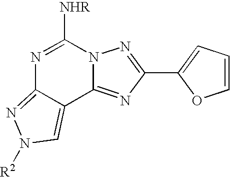

| Compound No | R² | R |
|---|---|---|
| 61d | H | 4-MeO-Ph-NHCO |
| 62d | H | 3-Cl-Ph-NHCO |
| 64d | t-C₄H₉ | 4-MeO-Ph-NHCO |
| 65d | t-C₄H₉ | 3-Cl-Ph-NHCO |
| 66d | CH₃ | Ph-NHCO |
| 67d | CH₃ | 4-SO₃H-Ph-NHCO |
| 68d | CH₃ | 3,4-Cl₂-Ph-NHCO |
| 69d | CH₃ | 3,4-(OCH₂—O)-Ph-NHCO |
| 70d | CH₃ | 4-(NO₂)-Ph-NHCO |
| 71d | CH₃ | 4-(CH₃)-Ph-NHCO |
| 72d | CH₃ | Ph-(CH₂)—CO |
| 73d | C₂H₅ | Ph-NHCO |
| 74d | C₂H₅ | 4-SO₃H-Ph-NHCO |
| 75d | C₂H₅ | 3,4-Cl₂-Ph-NHCO |
| 76d | C₂H₅ | 3,4-(OCH₂—O)-Ph-NHCO |
| 77d | C₂H₅ | 4-(NO₂)-Ph-NHCO |
| 78d | C₂H₅ | 4-(CH₃)-Ph-NHCO |
| 79d | C₂H₅ | Ph-(CH₂)—CO |
| 80d | n-C₃H₇ | Ph-NHCO |
| 81d | n-C₃H₇ | 4-SO₃H-Ph-NHCO |
| 82d | n-C₃H₇ | 3,4-Cl₂-Ph-NHCO |
| 83d | n-C₃H₇ | 3,4-(OCH₂—O)-Ph-NHCO |
| 84d | n-C₃H₇ | 4-(NO₂)-Ph-NHCO |
| 85d | n-C₃H₇ | 4-(CH₃)-Ph-NHCO |
| 86d | n-C₃H₇ | Ph-(CH₂)—CO |
| 87d | n-C₄H₉ | Ph-NHCO |
| 88d | n-C₄H₉ | 4-SO₃H-Ph-NHCO |
| 89d | n-C₄H₉ | 3,4-Cl₂-Ph-NHCO |
| 90d | n-C₄H₉ | 3,4-(OCH₂—O)-Ph-NHCO |
| 91d | n-C₄H₉ | 4-(NO₂)-Ph-NHCO |
| 92d | n-C₄H₉ | 4-(CH₃)-Ph-NHCO |
| 93d | 2-(α-napthyl)ethyl | Ph-(CH₂)—CO |
| 95d | 2-(α-napthyl)ethyl | 4-MeO-Ph-NHCO |
| 96d | 2-(α-napthyl)ethyl | 3-Cl-Ph-NHCO |
| 98d | 2-(2,4,5-tribromo- | 4-MeO-Ph-NHCO |
| | phenyl)ethyl | |
| 99d | 2-(2,4,5-tribromo- | 3-Cl-Ph-NHCO |
| | phenyl)ethyl | |
| 100d | 2-propen-1-yl | 4-MeO-Ph-NHCO |
| 108d | n-C₃H₇ | 4-MeO-Ph-NHCO |
| 109d | C₂H₅ | 4-MeO-Ph-NHCO |

6.1.2 Compounds of the General Formula IID

In another specific embodiment, the invention provides methods for treating a disease or disorder associated with HIF-1α in a patient, comprising administering to a patient in need thereof an effective amount of a compound or a pharmaceutically acceptable salt of the compound having the following general formula IID:

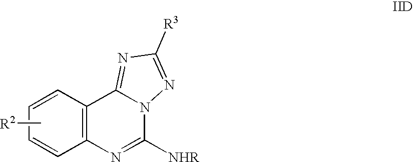

wherein:

R is —C(X)R¹, —C(X)—N(R¹)₂, —C(X)OR¹, —C(X)SR¹, —SO_{b}R¹, —SO_{b}OR¹, —SO_{b}SR¹, or —SO_{b}N(R¹)₂;

R¹ is hydrogen, alkyl, substituted alkyl, alkenyl, substituted alkenyl, alkynyl, substituted alkynyl, aryl, heteroaryl, heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms such as N, O, S;

R² is hydrogen, halogen, preferably chloro, alkyl, alkenyl, alkynyl, substituted alkyl, substituted alkenyl, substituted alkynyl, aralkyl, substituted aralkyl, aryl, substituted aryl, heteroaryl or substituted heteroaryl;

R³ is furan, pyrrole, thiophene, benzofuran, indole, benzothiophene, optionally substituted with one or more substituents as described herein for substituted heteroaryl rings;

X is O, S, or NR¹; and

b is 1 or 2.

In a specific embodiment the compound is of the formula IID as described above, with the proviso that R² is not halogen when R is —C(X)R¹. A preferred R group is —C(X)—N(R¹)₂, where X is O.

For compounds of the general formula IID, preferably, R¹ is hydrogen; C₁₋₈ alkyl; C₂₋₇ alkenyl, C₂₋₇ alkynyl; C₃₋₇ cycloalkyl; C₁₋₅ alkyl substituted with one or more halogen atoms, hydroxy groups, C₁₋₄ alkoxy, C₃₋₇ cycloalkyl or groups of formula —NR³², —C(O)NR³²; aryl, substituted aryl wherein the substitution is selected from the group consisting of C₁₋₄ alkoxy, C₁₋₄ alkyl, nitro, amino, cyano, C₁₋₄ haloalkyl, C₁₋₄ haloalkoxy, carboxy, carboxyamido; C₇₋₁₀ aralkyl in which the aryl moiety can be substituted with one or more of the substituents indicated above for the aryl group; a group of formula —(CH₂)ₘ-Het, wherein Het is a 5-6 membered aromatic or non aromatic heterocyclic ring containing one or more heteroatoms selected from the group consisting of N, O, and S and m is an integer from 1 to 5;

For compounds of the general formula IID, preferred C₁₋₈ alkyl groups are methyl, ethyl, propyl, butyl and isopentyl. Examples of C₃₋₇ cycloalkyl groups include cyclopropyl, cyclopentyl, and cyclohexyl. Examples of C₁₋₅ alkyl groups substituted with C₃₋₇ cycloalkyl groups include cyclohexylmethyl, cyclopentylmethyl, and 2-cyclopentylethyl. Examples of substituted C₁₋₅ alkyl groups include 2-hydroxyethyl, 2-methoxyethyl, trifluoromethyl, 2-fluoroethyl, 2-chloroethyl, 3-aminopropyl, 2-(4methyl-1-piperazine)ethyl, 2-(4-morpholinyl)ethyl, 2-aminocarbonylethyl, 2-dimethylaminoethyl, 3-dimethylaminopropyl. Aryl is preferably phenyl, optionally substituted with Cl, F, methoxy, nitro, cyano, methyl, trifluoromethyl, difluoromethoxy groups. Examples of 5 to 6 membered ring heterocyclic groups containing N, O and/or S include piperazinyl, morpholinyl, thiazolyl, pyrazolyl, pyridyl, furyl, thienyl, pyrrolyl, triazolyl, tetrazolyl. Examples of C₇₋₁₀ aralkyl groups comprise benzyl or phenethyl optionally substituted by one or more substituents selected from Cl, F, methoxy, nitro, cyano, methyl, trifluoromethyl, and difluoromethoxy. Preferably, R¹ is hydrogen, C₁₋₈ alkyl, aryl or C₇₋₁₀ aralkyl, optionally substituted, preferably with halogen atoms. Preferably, X is O, R² is chloro, C₂₋₃ alkyl or substituted alkyl and R³ is furan.

Particularly preferred compounds are those in which R is a phenethyl group in which the phenyl ring is substituted with one or more substituents selected from the group consisting of chlorine, fluorine atoms, methoxy, nitro, cyano, methyl, trifluoromethyl, and difluoromethoxy groups.

Non-limiting examples of compounds of the formula IID include compounds listed below in Table.

TABLE 4

| # | Compound |
|---|---|
| 1b | 5-[[4-methoxyphenyl)amino]carbonyl]amino-9-chloro-2-(2-furyl)- |
| | 1,2,4-triazolo[1,5-bc]quinazoline |
| 2b | 5-[[3-chlorophenyl)amino]carbonyl]amino-9-chloro-2-(2-furyl)- |
| | 1,2,4-triazolo[1,5-c]quinazoline |

6.2 Methods of Preparing the Compounds of the Invention

Compounds of the general formulas IIA, IIB and IIC can be prepared, for example, as described in U.S. Pat. Nos. 6,407,236; 6,448,253 and U.S. patent application Ser. No. 10/134,219, filed Apr. 26, 2002; the disclosures of which are hereby incorporated by reference in their entireties. Compounds of the general formula IID can be prepared, for example, as described in U.S. Pat. No. 6,358,964; the disclosure of which is hereby incorporated by reference in its entirety.

Where necessary, certain synthetic intermediates used to prepare compounds used in the invention may contain reactive moeities. Those skilled in the art of organic chemistry will appreciate that reactive and fragile functional groups often must be protected prior to a particular reaction, or sequence of reactions, and then restored to their original forms after the last reaction is completed. Usually groups are protected by converting them to a relatively stable derivative. For example, a hydroxyl group may be converted to an ether group and an amine group converted to an amide or carbamate. Methods of protecting and de-protecting, also known as “blocking” and “de-blocking,” are well known and widely practiced in the art, e.g., see T. Green, Protective Groups in Organic Synthesis, John Wiley, New York (1981) or Protective Groups in Organic Chemistry, Ed. J. F. W. McOmie, Plenum Press, London (1973).

6.3 Prophylactic and Therapeutic Methods

The present invention relates to methods for the treatment, prevention, and/or management of diseases or disorders associated with overexpression of HIF-1α and/or increased HIF-1α activity (e.g., cancer, respiratory disorders such as asthma and obstructive pulmonary disorders) by using A₃ receptor antagonists alone or in combination with A₁ receptor antagonists.

The methods and compositions of the invention comprising A₃ receptor antagonists are particularly useful when the levels of HIF-1α expression and/or activity are elevated above the standard or background level, as determined using methods known to those skilled in the art and dislosed herein. For example, elevation of the measured level relative to a standard level may be any statistically significant detectable elevation. Such an elevation of HIF-1α expression and/or activity may include, but is not limited to about a 10%, about a 20%, about a 40%, about an 80%, about a 2-fold, about a 4-fold, about a 10-fold, about a 20-fold, about a 50-fold, about a 100-fold, about a 2 to 20 fold, 2 to 50 fold, 2 to 100 fold, 20 to 50 fold, 20 to 100 fold, elevation, relative to the standard. The term “about” as used herein, refers to levels of elevation of the standard numerical value plus or minus 10% of the numerical value.

The therapeutic methods of the invention comprising administering a therapeutically effective amount of an A₃ receptor antagonist of the invention improve the therapeutic efficacy of treatment for diseases or disorders associated with overexpression of HIF-1α and/or increased HIF-1α activity (e.g., cancer, respiratory disorders such as asthma and obstructive pulmonary disorders) relative to the traditional modes of such therapies. Preferably the methods of the invention reduce the HIF-1α level to the background level within 1 day, 1 week, 1 month, at least 2 months of the commencement of the therapeutic regime. In a most preferred embodiment, the methods of the invention result in a reduction of an HIF-1α level to the background level. The invention encompasses reduction of the HIF-1α level to a level which is within about 10%, about 20%, about 30%, about 40%, about 50% of the background level.

In a preferred specific embodiment, the invention encompasses a method for treatment, prevention and/or management of diseases or disorders associated with overexpression of HIF-1α and/or increased HIF-1α activity (e.g., cancer, respiratory disorders such as asthma and obstructive pulmonary disorders) comprising administering a therapeutically and/or prophylactically effective amount of an A₃ receptor antagonist compound as disclosed herein.

Compounds of the present invention that function as prophylactic and/or therapeutic agents of a disease or disorder can be administered to an animal, preferably a mammal, and most preferably a human, to treat, prevent or ameliorate one or more symptoms associated with the disease or disorder. The subject is preferably a mammal such as non-primate (e.g., cows, pigs, horses, cats, dogs, rats, etc.) and a primate (e.g., monkey, such as a cynomolgous monkey and a human). In a preferred embodiment, the subject is a human. Compounds of the invention can be administered in combination with one or more other prophylactic and/or therapeutic agents useful in the treatment, prevention or management of a HIF-1α-mediated disorders, which are improved or ameliorated by modulation of HIF-1 expression or activities.

The amount of the compounds of the invention (including adenosine A₁ and/or A₃ antagonists) required to be effective will, of course, vary with the individual mammal being treated and is ultimately at the discretion of the medical or veterinary practitioner. The factors to be considered include the condition being treated, the route of administration, the nature of the formulation, the mammal's body weight, surface area, age and general condition, and the particular compound to be administered. However, a suitable effective dose is in the range of about 0.1 μg/kg to about 100 mg/kg, about 0.1 μg/kg to about 500 mg/kg, about 0.1 μg/kg to about 1 g/kg, about 100 ug/kg to about 500 mg/kg, about 100 ug/kg to about 1 g/kg, about 1 mg/kg to about 100 mg/kg, about 1 mg/kg to about 500 mg/kg, about 1 mg/kg to about 1 g/kg of the patient's body weight.

The total daily dose may be given as a single dose, multiple doses, e.g., two to six times per day, or by intravenous infusion for a selected duration. Dosages above or below the range cited above are within the scope of the present invention and may be administered to the individual patient if desired and necessary.

The methods and compositions of the invention comprise the administration of one or more compounds of the invention to subjects/patients suffering from or expected to suffer from a disease or disorder.

6.3.1 Combination Therapy

The invention encompasses combination therapies by administration of one or more compounds of the invention in combination with administration of one or more other therapies that are traditionally used for the treatment and/or prevention of the particular disease or disorder being treated or prevented. For example in the case of cancer, the A₃ and/or A₁ receptor antagonists of the invention may be administered in combination with one or more cancer therapies such as, but not limited to, chemotherapies, radiation therapies, hormonal therapies, and/or biological therapies/immunotherapies.

Prophylactic and therapeutic compounds that may be used in the methods and compositions of the invention include, but are not limited to, proteinaceous molecules, including, but not limited to, peptides, polypeptides, proteins, including post-translationally modified proteins, antibodies, etc.; small molecules (less than 1000 daltons), inorganic or organic compounds; nucleic acid molecules including, but not limited to, double-stranded or single-stranded DNA, double-stranded or single-stranded RNA, as well as triple helix nucleic acid molecules. Prophylactic and therapeutic compounds can be derived from any known organism (including, but not limited to, animals, plants, bacteria, fungi, and protista, or viruses) or from a library of synthetic molecules. In certain embodiments, one or more compounds of the invention are administered to a mammal, preferably a human, concurrently with one or more other therapeutic agents useful for the treatment of cancer or a disorder. The term “concurrently” is not limited to the administration of prophylactic or therapeutic agents at exactly the same time, but rather it is meant that compounds of the invention and the other agent are administered to a subject in a sequence and within a time interval such that the compounds of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician's Desk Reference (58th ed., 2004).

6.3.2 Antagonist-Based Therapy/Prophylaxis

6.3.2.1 Cancers

Compounds of the invention, particularly the A₁ and A₃ receptor antagonists, can be used alone or in combination with other anti-cancer agents, e.g., therapeutic antibodies known in the art to prevent, inhibit or reduce the growth of primary tumors or metastasis of cancerous cells. In a specific embodiment, a compound of the invention, when administered alone or in combination with an anti-cancer agent, inhibits or reduces the growth of primary tumor or metastasis of cancerous cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth of primary tumor or metastasis in absence of said compound of the invention. In a preferred embodiment, compounds of the invention particularly the A₁ and A₃ receptor antagonists in combination with an anti-cancer agent inhibit or reduce the growth of primary tumor or metastasis of cancer by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth or metastasis in absence of said compounds. The methods of the invention are particularly useful for the treatment of solid tumors, hypoxic tumors, A₃ expressing tumors and HIF-1α expressing tumors.

The compounds of the invention particularly the A₁ and A₃ receptor antagonists can be used to treat solid tumors. Solid tumors which can be treated in accordance with the methods of the invention include without limitation fibrodysplasia ossificans progressiva, prostate cancer, benign prostatic hyperplasia, recessive dystrophic epidermolysis bullosa (RDEB), Lewis lung carcinoma, breast cancer, and brain tumors.

As used herein, the term “A₃ expressing cancers” refers to human cancers that express the adenosine A₃ receptor or that otherwise comprise elevated concentrations of adenosine A₃ receptors. Elevated concentration is determined by comparison to normal, non-cancerous tissues of a similar cell type. Examples of A₃ expressing cancers include, without limitation, human leukemia, melanoma, pancreatic carcinoma, breast carcinoma, prostrate carcinoma, colon carcinoma, lung carcinomamalignant melanomas, histiocytic lymphoma, and some forms of astrocytoma cells.

As used herein the term “HIF-1α expressing cancers” refers to human cancer that express HIF-1α or that otherwise comprise elevated concentrations of HIF-1α. Elevated concentration is determined by comparison to normal, non-cancerous tissues of a similar cell type. Examples of HIF-1α expressing cancers include without limitation cervical cancer (early stage), lung cancer (non-small cell lung carcinoma), breast cancer (including lymph node positive breast cancer and lymph node negative breast cancer), oligodendroglioma, orpharyngeal squamous cell carcinoma, ovarian cancer, oesophageal cancer, endometrial cancer, head and neck cancer, gastrointestinal stromal tumor of the stomach.

In particular embodiments, the methods and compositions of the invention comprise the administration of one or more A₃ receptor antagonist compounds of the invention (alone or in combination with other anti-cancer agents) to subjects/patients suffering from or expected to suffer from cancer, e.g., have a genetic predisposition for a particular type of cancer, have been exposed to a carcinogen, or are in remission from a particular cancer. The methods and compositions of the invention may be used as a first line or second line cancer treatment. Included in the invention is also the treatment of patients undergoing other cancer therapies and the methods and compositions of the invention can be used before any adverse effects or intolerance of these other cancer therapies occurs. The invention also encompasses methods for administering one or more A₃ receptor antagonist compounds of the invention to treat or ameliorate symptoms in refractory patients. In a certain embodiment, that a cancer is refractory to a therapy means that at least some significant portion of the cancer cells are not killed or their cell division arrested. The determination of whether the cancer cells are refractory can be made either in vivo or in vitro by any method known in the art for assaying the effectiveness of treatment on cancer cells, using the art-accepted meanings of “refractory” in such a context. In various embodiments, a cancer is refractory where the number of cancer cells has not been significantly reduced, or has increased. The invention also encompasses methods for administering one or more A₃ receptor antagonist compounds of the invention to prevent the onset or recurrence of cancer in patients predisposed to having cancer.

In particular embodiments, the A₃ receptor antagonists of the invention are administered to reverse resistance or reduced sensitivity of cancer cells to certain hormonal, radiation and chemotherapeutic agents thereby resensitizing the cancer cells to one or more of these agents, which can then be administered (or continue to be administered) to treat or manage cancer, including to prevent metastasis.

In alternate embodiments, the invention provides methods for treating cancer in a subject by administering one or more A₃ receptor antagonists of the invention in combination with any other treatment or to patients who have proven refractory to other treatments but are no longer on these treatments. In certain embodiments, the patients being treated by the methods of the invention are patients already being treated with chemotherapy, radiation therapy, hormonal therapy, or biological therapy/immunotherapy. Among these patients are refractory patients and those with cancer despite treatment with existing cancer therapies. In other embodiments, the patients have been treated and have no disease activity and one or more A₃ receptor antagonist compounds of the invention are administered to prevent the recurrence of cancer.

Additionally, the invention also provides methods of treatment of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods of the invention may, optionally, be treated with other cancer treatments such as surgery, chemotherapy, radiation therapy, hormonal therapy or biological therapy, depending on which treatment was found to be unacceptable or unbearable.

In other embodiments, the invention provides administration of one or more A₃ receptor antagonist compounds of the invention without any other cancer therapies for the treatment of cancer, but who have proved refractory to such treatments. In specific embodiments, patients refractory to other cancer therapies are administered one or more A₃ receptor antagonist compounds in the absence of cancer therapies.

In a preferred embodiment when treating A₃ expressing cancers, a high affinity adenosine A₃ receptor antagonist and a chemotherapeutic cancer agent are administered to the patient. The combination therapy enhances the effect of the chemotherapeutic cancer agent and prevents multi-drug resistance from developing. Although not intending to be bound to a particular mechanism of action, the chemotherapeutic cancer agents showing desirable response to the present invention are typical of agents noted for developing P-gp or MRP class multi-drug resistance, including, without limitation, taxane compounds, vinca alkaloids, camptothecins and antibiotics useful as chemotherapeutic agents.

The invention encompasses combination therapies for treating cancers that have already developed multi-drug resistance. In this case, the high affinity adenosine A₃ receptor antagonist counters the existing MDR while further enhancing the effect of the chemotherapeutic cancer agent. As exemplified in the working examples, the present invention is not effective for all forms of MDR cancers. The MDR cancers showing desirable response to the present invention are in the P-gp and MRP classes.

When the chemotherapeutic cancer agents is a compound of the taxane family, the high affinity adenosine A₃ receptor antagonist is preferably administered either before or during administration of the taxane compound. Although not intending to be bound to a particular theory, this is done to reduce the incidence of hypersensitivity to the taxane agent. Commercially available compounds of the taxane family are paclitaxel (commercially available under the tradename TAXOL from Bristol-Myers Squibb Company, Princeton, N.Y. and as a generic drug from IVAX Corp, Miami, Fla.) and docetaxel (commercially available under the tradename TAXOTERE from Aventis Pharmaceuticals, Collegeville, Pa.), which are encompassed in the instant invention. It is noted that additional taxane family chemotherapeutic cancer agents are presently in development and testing and encompassed within the invention.

In another preferred embodiment for treating existing tumors, the composition comprises an effective amount of an adenosine A₃ receptor antagonist and a chemotherapeutic agent that is a taxane family compound, for example paclitaxel or docetaxel to inhibit tumor growth. Paclitaxel is commercially available under the tradename TAXOL from Bristol-Myers Squibb Company, Princeton, N.Y. and as a generic drug from IVAX Corp, Miami, Fla. Docetaxel is commercially available under the tradename TAXOTERE from Aventis Pharmaceuticals, Collegeville, Pa. In this embodiment the A₃ antagonist may reduce the growth rate of cancerous cells, interfere with adenosine protective effects against hypoxia, enhance the chemotherapeutic effect of the taxane, reduce hypersensitivity reactions in the patient and counter development of multi-drug resistance.

Cancers and related disorders that can be treated or prevented by methods and compositions comprising A₁ and/or A₃ receptor antagonists of the present invention include, but are not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma, gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, superficial spreading melanoma, nodular melanoma, lentigo malignant melanoma, acral lentiginous melanoma; kidney cancers including but not limited to, renal cell cancer, adenocarcinoma, hypernephroma, fibrosarcoma, transitional cell cancer (renal pelvis and/or uterer); Wilms' tumor; bladder cancers including but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other specific embodiments, sarcoma, melanoma, or leukemia is treated or prevented by the methods and compositions of the invention.

Cancers associated with the cancer antigens may be treated or prevented by administration of the A₃ receptor antagonists of the invention in combination with an antibody that binds the cancer antigen and is cytotoxic. In one particular embodiment, the A₃ receptor antagonists of the invention enhance the antibody mediated cytotoxic effect of the antibody directed at the particular cancer antigen. For example, but not by way of limitation, cancers associated with the following cancer antigen may be treated or prevented by the methods and compositions of the invention. KS 1/4 pan-carcinoma antigen (Perez and Walker, 1990, J. Immunol. 142:32-37; Bumal, 1988, Hybridoma 7(4):407-415), ovarian carcinoma antigen (CA125) (Yu et al., 1991, Cancer Res. 51(2):48-475), prostatic acid phosphate (Tailor et al., 1990, Nucl. Acids Res. 18(1):4928), prostate specific antigen (Henttu and Vihko, 1989, Biochem. Biophys. Res. Comm. 10(2):903-910; Israeli et al., 1993, Cancer Res. 53:227-230), melanoma-associated antigen p97 (Estin et al., 1989, J. Natl. Cancer Instit. 81(6):445-44), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-3; Mittelman et al., 1990, J. Clin. Invest. 86:2136-2144)), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, Colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reffet al., 1994, Blood 83:435-445), CD33 (Sgouros et al., 1993, J. Nucl. Med. 34:422-430), melanoma specific antigens such as ganglioside GD2 (Saleh et al., 1993, J.Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erthrocytes and primary endoderm, I(Ma) found in gastric adencarcinomas, M18 and M39 found in breast epithelium, SSEA-1 found in myeloid cells, VEP8, VEP9, My1, VIM-D5,and D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma, CO-514 (blood group Le^{a}) found in adenocarcinoma, NS-10 found in adenocarcinomas, CO-43 (blood group Le^{b}), G49, EGF receptor, (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, M1:22:25:8 found in embryonal carcinoma cells and SSEA-3, SSEA-4 found in 4-8-cell stage embryos. In another embodiment, the antigen is a T cell receptor derived peptide from a cutaneous T cell lymphoma (see Edelson, 1998, The Cancer Journal 4:62).

6.3.2.1.1 Other Agents

The high affinity adenosine A₃ receptor antagonists of the invention can be administered alone or in combination with other therapeutic and/or prophylactic agents.

The methods of the invention encompass the administration of one or more chemotherapeutic cancer agents. Chemotherapeutic cancer agents are defined as agents that attack and kill cancer cells. Chemotherapeutic agents that may be used in the methods of the invention include taxane compounds and derivatives thereof, paclitaxel and its derivatives or docetaxel and its derivatives. Additional taxane derivatives and methods of synthesis are disclosed in U.S. Pat. No. 6,191,287 to Holton et al., U.S. Pat. No. 5,705,508 to Ojima et al., U.S. Pat. Nos. 5,688,977 and 5,750,737 to Sisti et al., U.S. Pat. No. 5,248,796 to Chen et al., U.S. Pat. No. 6,020,507 to Gibson et al., U.S. Pat. No. 5,908,835 to Bissery, all of which are incorporated herein by reference in their entireties.

Other chemotherapeutic cancer agents that may be used in the methods of the invention include mitotic inhibitors (vinca alkaloids). These include vincristine, vinblastine, vindesine and Navelbine™ (vinorelbine,5′-noranhydroblastine). In yet other embodiments, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that may be used in the methods and compositions of the invention are podophyllotoxin derivatives, such as etoposide, teniposide and mitopodozide. The invention further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The invention encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions of the invention include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The invention further encompasses other chemotherapeutic cancer agents including without limitation anti-tumor antibodies, dacarbazine, azacytidine, amsacrine, melphalan, ifosfamide and mitoxantrone.

The A₃ antagonists can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

In one embodiment, the anti-angiogenesis compound is an adenosine A₂ₐ antagonist. A₂ₐ antagonists are well known to those of skill in the art. Examples of these compounds include SCH 58261 from Schering Plough, ZM241385 (Palmer et al., Mol. Pharmacol., 48:970-974 (1995), the tricyclic non-xanthine antagonists and the triazoloquinazolines, including CP 66,713, disclosed in Nikodijevic et al., J. Pharmacol. Exp. Ther., 259:286-294 (1991), as well as the compounds disclosed in von Lubitz et al., Eur. J. Pharmacol., 287:295-302 (1995). The contents of each of the references above are hereby incorporated by reference.

Anti-cancer agents that can be used in combination with A₃ receptor antagonists of the invention in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Examples of therapeutic antibodies that can be used in methods of the invention include but are not limited to HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Other examples of therapeutic antibodies that can be used in combination with the A₃ receptor antagonists of the invention are presented in Table 5 below:

TABLE 5
MONOCLONAL ANTIBODIES FOR CANCER THERAPY THAT CAN BE USED IN
COMBINATION WITH THE A₃ RECEPTOR ANTAGONISTS OF THE INVENTION.

| Company | Product | Disease | Target |
|---|---|---|---|
| Abgenix | ABX-EGF | Cancer | EGF receptor |
| AltaRex | OvaRex | ovarian cancer | tumor antigen CA125 |
| | BravaRex | metastatic cancers | tumor antigen MUC1 |
| Antisoma | Theragyn (pemtumomabytrrium-90) | ovarian cancer | PEM antigen |
| | Therex | breast cancer | PEM antigen |
| Boehringer Ingelheim | blvatuzumab | head & neck cancer | CD44 |
| Centocor/J&J | Panorex | Colorectal cancer | 17-1A |
| | ReoPro | PTCA | gp IIIb/IIIa |
| | ReoPro | Acute MI | gp IIIb/IIIa |
| | ReoPro | Ischemic stroke | gp IIIb/IIIa |
| Corixa | Bexocar | NHL | CD20 |
| CRC Technology | MAb, idiotypic 105AD7 | colorectal cancer vaccine | gp72 |
| Crucell | Anti-EpCAM | cancer | Ep-CAM |
| Cytoclonal | MAb, lung cancer | non-small cell lung | NA |
| | | cancer | |
| Genentech | Herceptin | metastatic breast cancer | HER-2 |
| | Herceptin | early stage breast cancer | HER-2 |
| | Rituxan | Relapsed/refractory low- | CD20 |
| | | grade or follicular NHL | |
| | Rituxan | intermediate & high- | CD20 |
| | | grade NHL | |
| | MAb-VEGF | NSCLC, metastatic | VEGF |
| | MAb-VEGF | Colorectal cancer, | VEGF |
| | | metastatic | |
| | AMD Fab | age-related macular | CD18 |
| | | degeneration | |
| | E-26 (2^{nd} gen. IgE) | allergic asthma & rhinitis | IgE |
| IDEC | Zevalin (Rituxan + yttrium-90) | low grade of follicular, | CD20 |
| | | relapsed or refractory, | |
| | | CD20-positive, B-cell | |
| | | NHL and Rituximab- | |
| | | refractory NHL | |
| ImClone | Cetuximab + innotecan | refractory colorectal | EGF receptor |
| | | carcinoma | |
| | Cetuximab + cisplatin & radiation | newly diagnosed or | EGF receptor |
| | | recurrent head & neck | |
| | | cancer | |
| | Cetuximab + gemcitabine | newly diagnosed | EGF receptor |
| | | metastatic pancreatic | |
| | | carcinoma | |
| | Cetuximab + cisplatin + 5FU or | recurrent or metastatic | EGF receptor |
| | Taxol | head & neck cancer | |
| | Cetuximab + carboplatin + paclitaxel | newly diagnosed non- | EGF receptor |
| | | small cell lung carcinoma | |
| | Cetuximab + cisplatin | head & neck cancer | EGF receptor |
| | | (extensive incurable | |
| | | local-regional disease & | |
| | | distant metasteses) | |
| | Cetuximab + radiation | locally advanced head & | EGF receptor |
| | | neck carcinoma | |
| | BEC2 + Bacillus Calmette Guerin | small cell lung carcinoma | mimics ganglioside GD3 |
| | BEC2 + Bacillus Calmette Guerin | melanoma | mimics ganglioside GD3 |
| | IMC-1C11 | colorectal cancer with | VEGF-receptor |
| | | liver metasteses | |
| ImmonoGen | nuC242-DM1 | Colorectal, gastric, and | nuC242 |
| | | pancreatic cancer | |
| ImmunoMedics | LymphoCide | Non-Hodgkins | CD22 |
| | | lymphoma | |
| | LymphoCide Y-90 | Non-Hodgkins | CD22 |
| | | lymphoma | |
| | CEA-Cide | metastatic solid tumors | CEA |
| | CEA-Cide Y-90 | metastatic solid tumors | CEA |
| | CEA-Scan (Tc-99m-labeled | colorectal cancer | CEA |
| | arcitumomab) | (radioimaging) | |
| | CEA-Scan (Tc-99m-labeled | Breast cancer | CEA |
| | arcitumomab) | (radioimaging) | |
| | CEA-Scan (Tc-99m-labeled | lung cancer | CEA |
| | arcitumomab) | (radioimaging) | |
| | CEA-Scan (Tc-99m-labeled | intraoperative tumors | CEA |
| | arcitumomab) | (radio imaging) | |
| | LeukoScan (Tc-99m-labeled | soft tissue infection | CEA |
| | sulesomab) | (radioimaging) | |
| | LymphoScan (Tc-99m-labeled) | lymphomas | CD22 |
| | | (radioimaging) | |
| | AFP-Scan (Tc-99m-labeled) | liver 7 gem-cell cancers | AFP |
| | | (radioimaging) | |
| Intracel | HumaRAD-HN (+yttrium-90) | head & neck cancer | NA |
| | HumaSPECT | colorectal imaging | NA |
| Medarex | MDX-101 (CTLA-4) | Prostate and other | CTLA-4 |
| | | cancers | |
| | MDX-210 (her-2 overexpression) | Prostate cancer | HER-2 |
| | MDX-210/MAK | Cancer | HER-2 |
| MedImmune | Vitaxin | Cancer | ανβ3 |
| Merck KGaA | MAb 425 | Various cancers | EGF receptor |
| | IS-IL-2 | Various cancers | Ep-CAM |
| Millennium | Campath (alemtuzumab) | chronic lymphocytic | CD52 |
| | | leukemia | |
| NeoRx | CD20-streptavidin (+biotin-yttrium | Non-Hodgkins | CD20 |
| | 90) | lymphoma | |
| | Avidicin (albumin + NRLU13) | metastatic cancer | NA |
| Peregrine | Oncolym (+iodine-131) | Non-Hodgkins | HLA-DR 10 beta |
| | | lymphoma | |
| | Cotara (+iodine-131) | unresectable malignant | DNA-associated proteins |
| | | glioma | |
| Pharmacia Corporation | C215 (+staphylococcal enterotoxin) | pancreatic cancer | NA |
| | MAb, lung/kidney cancer | lung & kidney cancer | NA |
| | nacolomab tafenatox (C242 + staphylococcal | colon & pancreatic | NA |
| | enterotoxin) | cancer | |
| Protein Design Labs | Nuvion | T cell malignancies | CD3 |
| | SMART M195 | AML | CD33 |
| | SMART 1D10 | NHL | HLA-DR antigen |
| Titan | CEAVac | colorectal cancer, | CEA |
| | | advanced | |
| | TriGem | metastatic melanoma & | GD2-ganglioside |
| | | small cell lung cancer | |
| | TriAb | metastatic breast cancer | MUC-1 |
| Trilex | CEAVac | colorectal cancer, | CEA |
| | | advanced | |
| | TriGem | metastatic melanoma & | GD2-ganglioside |
| | | small cell lung cancer | |
| | TriAb | metastatic breast cancer | MUC-1 |
| Viventia Biotech | NovoMAb-G2 radiolabeled | Non-Hodgkins | NA |
| | | lymphoma | |
| | Monopharm C | colorectal & pancreatic | SK-1 antigen |
| | | carcinoma | |
| | GlioMAb-H (+gelonin toxin) | gliorna, melanoma & | NA |
| | | neuroblastoma | |
| Xoma | Rituxan | Relapsed/refractory low- | CD20 |
| | | grade or follicular NHL | |
| | Rituxan | intermediate & high- | CD20 |
| | | grade NHL | |
| | ING-1 | adenomcarcinoma | Ep-CAM |

The A₃ receptor antagonists of the invention may be used in combination with one or more ATP depleting agents for enhancing the therapeutic efficacy of treating a disease or disorder as disclosed herein, i.e., cancer. Alternatively, the A₃ receptor antagonists of the invention may be used with verapamil for enhancing the therapeutic efficacy of conventional modes of treating a disease or disorder as discussed herein, i.e., cancer.

6.3.2.2 Respiratory Disorders

Compounds of the invention, particularly the A₁ and A₃ receptor antagonists, can be used alone or in combination with other therapeutic and/or prophylactic agents to prevent, treat or manage respiratory disorders such as asthma and obstructive pulmonary disorders (COPD).

The methods and compositions comprising A₁ and/or A₃ receptor antagonists of the invention are effective for treatment, prevention, and/or management of asthma. The therapeutic and prophylactic methods of the invention for asthma may be used in combination with other methods known in the art for the treatment, prevention and/or management of asthma including but not limited to inhaled beta 2 agonists, inhaled corticosteroids, retinoic acid, anti-IgE antibodies, phosphodiesterase inhibitors, leukotriene antagonists, anti IL-9 antibody, and/or anti-mucin therapies (e.g., anti hCLCA1 therapy such as Lomucin™). β-adrenergic drugs (e.g. epinephrine and isoproterenol), theophylline, anticholinergic drugs (e.g., atropine and ipratorpium bromide), and corticosteroids, adrenergic stimulants (e.g., catecholamines (e.g., epinephrine, isoproterenol, and isoetharine), resorcinols (e.g., metaproterenol, terbutaline, and fenoterol), and saligenins (e.g., salbutamol), other steroids, immunosuppressant agents (e.g., methotrexate and gold salts), mast cell modulators (e.g., cromolyn sodium (INTAL™) and nedocromil sodium (TILADE™)), and mucolytic agents (e.g., acetylcysteine)).

The methods and compositions comprising A₁ and/or A₃ receptor antagonists of the invention are effective for treatment, prevention, and/or management of COPD including fixed airways disorders, chronic bronchitis and emphysema.

The therapeutic and prophylactic methods of the invention may be used in combination with other therapeutic methods known in the art for the treatment, prevention and/or management of COPD including but not limited tiotropium and/or ipratropium

Other respiratory disorders that may be treated, prevented or managed using the methods and compositions of the invention include without limitation lung fibrosis, bronchial hyper responsiveness.

6.4 Compositions and Methods of Administering

The invention provides methods and pharmaceutical compositions comprising compounds of the invention. The invention also provides methods of treatment, prophylaxis, and amelioration of one or more symptoms associated with a disease, disorder or cancer by administering to a subject an effective amount of compound of the invention. In a specific embodiment, the subject is an animal, preferably a mammal such as non-primate (e.g., cows, pigs, horses, cats, dogs, rats etc.) and a primate (e.g., monkey such as, a cynomolgous monkey and a human). In a preferred embodiment, the subject is a human.

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of compounds of the invention and a pharmaceutically acceptable carrier.

In one particular embodiment, the pharmaceutical composition comprises a therapeutically or prophylactically effective amount of an A₃ or A₁ receptor antagonist and a pharmaceutically acceptable carrier. In another embodiment, said pharmaceutical composition further comprises one or more anti-cancer agents.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with captions such as those derived from sodium, potassium, ammonium, calcium, ferric hydroxides, isopropylamine, triethylamine, 2-ethylamino ethanol, histidine, procaine, etc.

The compounds described above are preferably administered in formulations including an active compound, i.e., an adenosine A₁ and/or A₃ receptor antagonist, together with an acceptable carrier for the mode of administration. Suitable pharmaceutically acceptable carriers are known to those of skill in the art. The compositions can optionally include other therapeutically active agents, including anti-angiogenic and anti-cancer agents. Other optional ingredients include antivirals, antibacterials, anti-inflammatories, analgesics, and immunosuppresants. The carrier must be pharmaceutically acceptable in the sense of being compatible with the other ingredients of the formulation and not deleterious to the recipient thereof.

The compounds of the invention, e.g., high affinity adenosine A₃ receptor antagonists, can be administered in a physiologically acceptable diluent in a pharmaceutical carrier, such as a sterile liquid or mixture of liquids, including water, saline, aqueous dextrose and related sugar solutions, an alcohol (e.g., ethanol, isopropanol, or hexadecyl alcohol), glycols (e.g., propylene glycol or polyethylene glycol), glycerol ketals, (e.g., 2,2-dimethyl1,3-dioxolane-4-methanol), ethers, (e.g., poly(ethyleneglycol) 400), an oil, a fatty acid, a fatty acid ester or glyceride, or an acetylated fatty acid glyceride with or without the addition of a pharmaceutically acceptable surfactant (e.g., a soap or a detergent), a suspending agent, such as pectin, carbomers, methylcellulose, hydroxypropylmethylcellulose, or carboxymethylcellulose, or emulsifying agents and other pharmaceutical excipients and adjuvants.

The formulations can include carriers suitable for oral, rectal, topical or parenteral (including subcutaneous, intramuscular and intravenous) administration. Preferred carriers are those suitable for oral or parenteral administration.

Formulations suitable for parenteral administration conveniently include sterile aqueous preparation of the active compound which is preferably isotonic with the blood of the recipient. Thus, such formulations may conveniently contain distilled water, 5% dextrose in distilled water or saline. Useful formulations also include concentrated solutions or solids containing the compounds which upon dilution with an appropriate solvent give a solution suitable for parental administration above.

Formulations suitable for parenteral administration include but are not limited to aqueous and non-aqueous solutions, isotonic sterile injection solutions, which may comprise anti-oxidants, buffers, bacteriostats, and solutes that render the formulation isotonic with the blood of the intended recipient, and aqueous and non-aqueous sterile suspensions that may comprise suspending agents, solubilizers, thickening agents, stabilizers, and preservatives.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and-tablets of the kind previously described.

The compounds of the invention, e.g., high affinity adenosine A₃ receptor antagonists may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986); which are incorporated herein by reference in their entireties.

For enteral administration, the compound can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch.

Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

A tablet may be made by compression or molding, optionally with one or more accessory ingredients. Compressed tablets may be prepared by compressing in a suitable in a machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

The compounds can also be administered locally by topical application of a solution, ointment, cream, gel, lotion or polymeric material (for example, a Pluronic™, BASF), which may be prepared by conventional methods known in the art of pharmacy. In addition to the solution, ointment, cream, gel, lotion or polymeric base and the active ingredient, such topical formulations may also contain preservatives, perfumes, and additional active pharmaceutical agents. Topical formulations for high affinity adenosine A₃ receptor antagonists include ointments, creams, gels and lotions that may be prepared by conventional methods known in the art of pharmacy. Such topical formulation may also furhter comprise preservatives, perfumes, and additional active pharmaceutical agents. Preferred additional pharmaceutical agents include the chemotherapeutic agents for cancer treatments noted to be enhanced or benefited by the A₃ receptor antagonist (for example by preventing MDR). An example of a preferred topical formulation includes a high affinity adenosine A₃ receptor antagonist and a taxane family compound.

Oils, which can be used in parenteral formulations include petroleum, animal, vegetable, and synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

Additionally, the compounds of the invention, e.g., high affinity adenosine A₃ receptor antagonist may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Formulations for rectal administration may be presented as a suppository with a conventional carrier, e.g., cocoa butter or Witepsol S55 (trademark of Dynamite Nobel Chemical, Germany), for a suppository base.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

In addition to the aforementioned ingredients, the formulations of this invention may further include one or more cytotoxic agent as well as one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

Various delivery systems are known and can be used to administer a composition comprising compounds of the invention, e.g., encapsulation in liposomes, microparticles, microcapsules.

In some embodiments, the compounds of the invention are formulated in liposomes for targeted delivery of the compounds of the invention. Liposomes are vesicles comprised of concentrically ordered phopsholipid bilayers which encapsulate an aqueous phase. Liposomes typically comprise various types of lipids, phospholipids, and/or surfactants. The components of liposomes are arranged in a bilayer configuration, similar to the lipid arrangement of biological membranes. Liposomes are particularly preferred delivery vehicles due, in part, to their biocompatibility, low immunogenicity, and low toxicity. Methods for preparation of liposomes are known in the art and are encompassed within the invention, see, e.g., Epstein et al., 1985, Proc. Natl. Acad. Sci. USA, 82: 3688; Hwang et al., 1980 Proc. Natl. Acad. Sci. USA, 77: 4030-4; U.S. Pat. Nos. 4,485,045 and 4,544,545; all of which are incorporated herein by reference in their entirety. The invention also encompasses methods of preparing liposomes with a prolonged serum half-life, i.e., enhanced circulation time, such as those disclosed in U.S. Pat. No. 5,013,556. Preferred liposomes used in the methods of the invention are not rapidly cleared from circulation, i.e., are not taken up into the mononuclear phagocyte system (MPS). The invention encompasses sterically stabilized liposomes which are prepared using common methods known to one skilled in the art. Although not intending to be bound by a particular mechanism of action, sterically stabilized liposomes contain lipid components with bulky and highly flexible hydrophilic moieties, which reduces the unwanted reaction of liposomes with serum proteins, reduces oposonization with serum components and reduces recognition by MPS. Sterically stabilized liposomes are preferably prepared using polyethylene glycol. For preparation of liposomes and sterically stabilized liposome see, e.g., Bendas et al., 2001 BioDrugs, 15(4): 215-224; Allen et al., 1987 FEBS Lett. 223: 42-6; Klibanov et al., 1990 FEBS Lett., 268: 235-7; Blum et al., 1990, Biochim. Biophys. Acta., 1029: 91-7; Torchilin et al., 1996, J. Liposome Res. 6: 99-116; Litzinger et al., 1994, Biochim. Biophys. Acta, 1190: 99-107; Maruyama et al., 1991, Chem. Pharm. Bull., 39: 1620-2; Klibanov et al., 1991, Biochim Biophys Acta, 1062; 142-8; Allen et al., 1994, Adv. Drug Deliv. Rev, 13: 285-309; all of which are incorporated herein by reference in their entirety. The invention also encompasses liposomes that are adapted for specific organ targeting, see, e.g., U.S. Pat. No. 4,544,545. Particularly useful liposomes for use in the compositions and methods of the invention can be generated by reverse phase evaporation method with a lipid composition comprising phosphatidylcholine, cholesterol, and PEG derivatized phosphatidylethanolamine (PEG-PE). Liposomes are extruded through filters of defined pore size to yield liposomes with the desired diameter. In some embodiments, a fragment of an antibody of the invention, e.g., F(ab′), may be conjugated to the liposomes using previously described methods, see, e.g., Martin et al., 1982, J. Biol. Chem. 257: 286-288, which is incorporated herein by reference in its entirety.

Methods for preparing liposomes and microspheres for administration to a patient are well known to those of skill in the art. U.S. Pat. No. 4,789,734, the contents of which are hereby incorporated by reference, describes methods for encapsulating biological materials in liposomes. essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 287-341 (Academic Press, 1979), which is incorporated herein by reference in its entirety.

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, the contents of which are hereby incorporated by reference.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In another embodiment, the compositions can be delivered in a vesicle, in particular a liposome (See Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 3 17-327; see generally ibid.).

In yet another embodiment, the compositions can be delivered in a controlled release or sustained release system. Any technique known to one of skill in the art can be used to produce sustained release formulations comprising one or more A3 receptor antagonists of the invention. See, e.g., U.S. Pat. No. 4,526,938; PCT publication WO 91/05548; PCT publication WO 96/20698; Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397; Cleek et al., 1997, “Biodegradable Polymeric Carriers for a bFGF Antibody for Cardiovascular Application,” Pro. Int'l. Symp. Control. Rel. Bioact. Mater. 24:853-854; and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in its entirety. In one embodiment, a pump may be used in a controlled release system (See Langer, supra; Sefton, 1987, CRC Crit. Ref. Biomed. Eng. 14:20; Buchwald et al., 1980, Surgery 88:507; and Saudek et al., 1989, N. Engl. J. Med. 321:574). In another embodiment, polymeric materials can be used to achieve controlled release of A3 receptor antagonists (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; See also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In yet another embodiment, a controlled release system can be placed in proximity of the therapeutic target (e.g., the lungs), thus requiring only a fraction of the systemic dose (see, e.g., Goodson, in Medical Applications of Controlled Release, supra, vol. 2, pp. 115-138 (1984)). In another embodiment, polymeric compositions useful as controlled release implants are used according to Dunn et al. (See U.S. Pat. No. 5,945,155). This particular method is based upon the therapeutic effect of the in situ controlled release of the bioactive material from the polymer system. The implantation can generally occur anywhere within the body of the patient in need of therapeutic treatment. In another embodiment, a non-polymeric sustained delivery system is used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (See U.S. Pat. No. 5,888,533). Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

Methods of administering a compound of the invention include, but are not limited to, parenteral administration (e.g., intradermal, intramuscular, intraperitoneal, intravenous and subcutaneous), epidural, and mucosal (e.g., intranasal and oral routes). In a specific embodiment, the compounds of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,20; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

In one embodiment, the compounds are administered intravenously in a liposome or microparticle with a size such that the particle can be delivered intraveneously, but gets trapped in a capillary bed around a growing tumor. Suitable particle sizes for this embodiment are those currently used, for example, in liposomes sold under the name DaunoXome.TM., which are believed to be between about 200 and 500 um. The compounds are then released locally, over time, at the location of the tumor. This can be effective in limiting systemic effects of the administration of cytotoxic agents, and also targets the adenosine antagonist and any other anti-angiogenic agents directly to the site at which blood vessel growth is to be inhibited.

In another embodiment, the compounds are administered in a tissue coating, preferably a polymeric tissue coating, more preferably, a biodegradable tissue coating, which is applied to the site at which a tumor is surgically removed. Suitable polymeric materials are disclosed, for example, in U.S. Pat. No. 5,410,016 to Hubbell et al., the contents of which are hereby incorporated by reference.

The polymeric barrier, in combination with the adenosine A3 antagonists, and optionally in combination with other anti-angiogenic agents and/or cytotoxic agents, provides a physical barrier as well as a chemical barrier to further tumor growth and vasculation around the tumor.

The two embodiments described above can be particularly useful when cytotoxic agents with pronounced systemic side effects are used. By targeting the administration of these compounds to a specific site, and locally delivering the compounds, systemic effects can be minimized.

In yet another embodiment, anti-VEGF antibodies can be covalently coupled to microparticles or liposomes including the compounds. The antibodies can be used to not only target the delivery of the compounds, but also to assist in inhibiting tumor growth by inhibiting angiogenesis.

The amount of the composition of the invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

Treatment of a subject with a therapeutically or prophylactically effective amount of compounds of the invention can include a single treatment or, preferably, can include a series of treatments. In a preferred example, a subject is treated with compounds of the invention in the range of between about 0.1 μg/kg to about 100 mg/kg, about 0.1 μg/kg to about 500 mg/kg, about 0.1 μg/kg to about 1 g/kg, about 100 ug/kg to about 500 mg/kg, about 100 ug/kg to about 1 g/kg, about 1 mg/kg to about 100 mg/kg, about 1 mg/kg to about 500 mg/kg, about 1 mg/kg to about 1 g/kg of the patient's body weight. one time per week for between about 1 to 10 weeks, preferably between 2 to 8 weeks, more preferably between about 3 to 7 weeks, and even more preferably for about 4, 5, or 6 weeks. In other embodiments, the pharmaceutical compositions of the invention are administered once a day, twice a day, or three times a day. In other embodiments, the pharmaceutical compositions are administered once a week, twice a week, once every two weeks, once a month, once every six weeks, once every two months, twice a year or once per year. It will also be appreciated that the effective dosage of the compounds used for treatment may increase or decrease over the course of a particular treatment. The amount of a compound required to be effective as an antagonist of adenosine A₃ receptors will, of course, vary with the active moiety selected, the individual mammal being treated and is ultimately at the discretion of the medical or veterinary practitioner. The factors to be considered include the binding affinity of the active, the route of administration, the nature of the formulation, the mammal's body weight, surface area, age and general condition, and the particular compound to be administered.

The total daily dose may be given as a single dose, multiple doses, e.g., two to six times per day, or by intravenous infusion for a selected duration. Dosages above or below the range cited above are within the scope of the present invention and may be administered to the individual patient if desired and necessary. For example, for a 75 kg mammal, a dose range would be about 75 ug to about 50 mg per day, and a typical dose would be about 50 mg per day. If discrete multiple doses are indicated, treatment might typically be 50 mg of a compound of the present invention given 3 times per day.

The invention also provides that the compounds of the invention are packaged in a hermetically sealed container such as an ampoule or sachette indicating the quantity of antibody. In one embodiment, the compounds of the invention are supplied as a dry sterilized lyophilized powder or water free concentrate in a hermetically sealed container and can be reconstituted, e.g., with water or saline to the appropriate concentration for administration to a subject. Preferably, the compounds of the invention are supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, at least 100 mg, at least 200 mg, at least 500 mg or at least 1 gram. The lyophilized compounds of the invention should be stored at between 2 and 8° C. in their original container and the compounds should be administered within 12 hours, preferably within 6 hours, within 5 hours, within 3 hours, or within 1 hour after being reconstituted. In an alternative embodiment, compounds of the invention are supplied in liquid form in a hermetically sealed container indicating the quantity and concentration of the compound. Preferably, the liquid form of the compounds are supplied in a hermetically sealed container at least 1 mg/ml, more preferably at least 2.5 mg/ml, at least 5 mg/ml, at least 8 mg/ml, at least 10 mg/ml, at least 15 mg/kg, at least 25 mg/ml, at least 50 mg/ml, at least 100 mg/ml, at least 150 mg/ml, at least 200 mg/ml of the compounds.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

In addition to the aforementioned ingredients, the formulations may further include one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

The formulations include, but are not limited to, those suitable for oral, rectal, topical or parenteral (including subcutaneous, intramuscular and intravenous) administration. Preferred are those suitable for oral or parenteral administration.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there are a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

The compounds of the invention, e.g., high affinity adenosine A₃ receptor antagonist, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

6.5 Characterization and Demonstration of Therapeutic/Prophylactic Utility

Several aspects of the pharmaceutical compositions, prophylactic or therapeutic agents of the invention are preferably tested in vitro, e.g., in a cell culture system, and then in vivo, e.g., in an animal model organism, such as a rodent animal model system, for the desired therapeutic activity prior to use in humans. For example, assays which can be used to determine whether administration of a specific pharmaceutical composition is indicated, include cell culture assays in which a patient tissue sample is grown in culture, and exposed to or otherwise contacted with a pharmaceutical composition, and the effect of such composition upon the tissue sample is observed, e.g., inhibition of or decrease in growth and/or colony formation in soft agar or tubular network formation in three-dimensional basement membrane or extracellular matrix preparation. The tissue sample can be obtained by biopsy from the patient. This test allows the identification of the therapeutically most effective prophylactic or therapeutic molecule(s) for each individual patient. Alternatively, instead of culturing cells from a patient, therapeutic agents and methods may be screened using cells of a tumor or malignant cell line. In various specific embodiments, in vitro assays can be carried out with representative cells of cell types involved in an autoimmune or inflammatory disorder (e.g., T cells), to determine if a pharmaceutical composition of the invention has a desired effect upon such cell types. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, decreased growth and/or colony formation in soft agar or tubular network formation in three-dimensional basement membrane or extracellular matrix preparation.

Combinations of prophylactic and/or therapeutic agents can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, combinations of prophylactic and/or therapeutic agents are tested in a mouse model system. Such model systems are widely used and well-known to the skilled artisan. Prophylactic and/or therapeutic agents can be administered repeatedly. Several aspects of the procedure may vary such as the temporal regime of administering the prophylactic and/or therapeutic agents, and whether such agents are administered separately or as an admixture.

Once the prophylactic and/or therapeutic agents of the invention have been tested in an animal model they can be tested in clinical trials to establish their efficacy. Establishing clinical trials will be done in accordance with common methodologies known to one skilled in the art, and the optimal dosages and routes of administration as well as toxicity profiles of the compositions of the invention can be established using routine experimentation.

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-cancer activity of the therapies used in accordance with the present invention also can be determined by using various experimental animal models for the study of cancer such as the SCID mouse model or transgenic mice or nude mice with human xenografts, animal models, such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

The protocols and compositions of the invention are preferably tested in vitro, and then in vivo, for the desired therapeutic or prophylactic activity, prior to use in humans. Therapeutic agents and methods may be screened using cells of a tumor or malignant cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, decreased growth and/or colony formation in soft agar or tubular network formation in three-dimensional basement membrane or extracellular matrix preparation, etc. Compounds for use in therapy can be tested in suitable animal model systems prior to testing in humans, including but not limited to in rats, mice, chicken, cows, monkeys, rabbits, hamsters, etc., for example, the animal models described above. The compounds can then be used in the appropriate clinical trials.

6.5.1 Anti-Proliferative Assays

The anti-proliferative activity of compounds of the invention can be readily determined using no more than routine experimentation using cell growth inhibitory assays. Selection of cell lines for such assays is based upon desired future pharmaceutical use. Numerous cell lines are available for study from American Type Culture Collection, Manassas, Va. Cell lines suitable for assays include, but are not limited to, HL-60 human leukemia, A375 human melanoma, SKMES human lung carcinoma, HT29 human colon carcinoma and Panc-1 human pancreatic carcinoma. Cell lines can be maintained in RPMI-1640 medium supplemented with 100 units/ml penicillin G sodium, 100 μg/ml streptomycin sulfate, 0.25 μg/ml amphotericin B (fuhgizone), 2 mM glutamine, 10 mM HEPES, 25 μg/ml gentamycin, and 10% heat-inactivated fetal bovine serum. Such maintained cells can be cultured in a T25 Falcon Tissue Culture flask in a humidified incubator at 37° C. with 5% CO₂-95% air. For example, such maintained cells can be sub-cultured twice a week with approximate doubling times of 22 hours for A375 human melanoma, 46 hours for SKMES human lung carcinoma, 48 hours for HT29 human colon carcinoma and 60 hours for Panc-1 human pancreatic carcinoma.

One of the growth inhibitory assays that can be used in accordance with the methods of the invention is the MTT assay. An exemplary assay may comprise the following steps: Cells (1000-1500 cells/well) are seeded in a 96-well micro culture plate in a total volume of 100 μl/well. After overnight incubation in a humidified incubator at 37° C. with 5% CO₂-95% air, chemotherapeutic drug solutions diluted with culture medium at various concentrations are added in the amount of 100 μl to each well. The plates are placed in a humidified incubator at 37° C. with 5% CO₂-95% air for 7-10 days. The plates are then centrifuged briefly and 100 μl of the growth medium is removed. Cell cultures are incubated with 50 μl of MTT [3-(4,5-dimethylthiazol-2-yl)-2,5-diphenyl-tetrazolium bromide] reagent (1 mg/ml in Dulbecco's phosphate-buffered saline) for 4 hr at 37° C. The resultant purple formazan precipitate is solubilized with 200 μl of 0.04 N HCl in isopropanol. Absorbance is measured at a wavelength of 595 nm and at a reference wavelength of 655 nm using a Bio-Rad Model 3550 Microplate Reader. Preferably, all tests are run in duplicate for each dose level. Bio-Rad brands Microplate Readers, when properly equipped, transmit measured test results to a personal computer for interpretation via computer programs such as the EZED50 program. The EZED50 computer program estimates the concentration of agent that inhibits cell growth by 50% as compared to the control cells. This is termed the IC₅₀ and is determined by curve fitting test data using the following four logistic equation.

$$Y = \frac{A max - A min}{1 + {( X / {IC}_{50} )}^{n}} + A min$$

where Aₘₐₓ is the absorbance of the control cells, Aₘᵢₙ is the absorbance of the cells in the presence of the highest agent concentration, Y is the observed absorbance, X is the agent concentration, IC₅₀ is the concentration of agent that inhibits the cell growth by 50% compared to the control cells, and n is the slope of the curve.

If testing concentrations are properly selected (i.e., no or little growth inhibition at low concentrations and complete inhibition at high concentrations), EZED50 program fits the data extremely well and estimates the IC₅₀ value accurately. If the testing agent was too potent and inhibits cell growth by more than 50% at all of the concentrations tested, EZED50 cannot estimate the IC₅₀ value accurately (as indicated by an erroneously fitted Amax value). In these instances, the data could be re-analyzed by fixing the Amax value.

On the other hand, if the compound being tested produces incomplete inhibition at the highest agent concentration, EZED50 will overestimate the potency of the testing agent (the fitted IC₅₀value is lower than the “actual” IC₅₀ value). In this case, the IC₅₀ could be estimated more accurately if both the Aₘₐₓ and Aₘᵢₙ values are fixed. Although it may be feasible to estimate IC₅₀ value by fixing Amax and/or Aₘₐₓ and Aₘᵢₙ without repeating the experiment, the best way to determine the IC₅₀ accurately is to decrease or increase the concentrations of the test agent and repeat the test.

Inhibitory assay testing is also used to determine enhanced therapeutic effects from combining A₃ receptor antagonists with other tumor inhibiting agents. The inhibitory cell growth assays are run with and without selected concentrations of A₃ receptor antagonists. An enhanced therapeutic effect is present when the IC₅₀ of the tumor inhibiting agent is lower with the A₃ receptor antagonist.

Quantitatively, the enhancement factor (EF) is calculated by dividing the IC₅₀ value of the tumor inhibiting agent for a tumor cell line by the IC₅₀ value of the agent with A₃ receptor antagonist for the same cell line:

$$Enhanced Factor ( EF ) = \frac{{IC}_{50} of anti - tumor agent}{\begin{matrix}{IC}_{50} of anti - tumor \\ agent with {A}_{3} receptor antagonist\end{matrix}}$$

Interpretation of the EF is dependent upon the concentrations of A₃ receptor antagonist tested. For example, when using very low concentrations of A₃ receptor antagonist, any EF above 1.0 is synergistic if the A₃ receptor antagonist concentration is below the threshold of cell growth inhibition.

6.5.2 Adenosine Receptor Based Assays

The activity and selectivity of the compounds of the invention as adenosine A₃ antagonists can be readily determined using no more than routine experimentation using any of the assays disclosed herein or known to one skilled in the art. Since the A₁ and A₂ₐ receptors express similar pharmacology between humans and rodents, endogenous receptors from the rat can be used for the A₁ and A₂ₐ binding assays.

An exemplary rat A₁ and A_{2A} Adenosine receptor binding assay may comprise the following steps. Membrane preparations: Male Wistar rats (200-250 g) can be decapitated and the whole brain (minug brainstem, striatum and cerebellum) dissected on ice. The brain tissues can be disrupted in a Polytron (setting 5) in 20 vols of 50 mM Tris HCl, pH 7.4. The homogenate can then be centrifuged at 48,000 g for 10 min and the pellet resuspended in Tris-HCL containing 2 IU/ml adenosine deaminase, type VI (Sigma Chemical Company, St. Louis, Mo., USA). After 30 min incubation at 37° C., the membranes can be centrifuged and the pellets stored at −70° C. Striatal tissues can be homogenized with a Polytron in 25 vol of 50 mM Tris HCl buffer containing 10 mM MgCl₂ pH 7.4. The homogenate can then be centrifuged at 48,000 g for 10 min at 4° C. and resuspended in Tris HCl buffer containing 2 IU/ml adenosine deaminase. After 30 min incubation at 37° C., membranes can be centrifuged and the pellet stored at −70° C. The radioligand binding assays may comprise the following: Binding of [³H]-DPCPX (1,3-dipropyl-8-cyclopentylxanthine) to rat brain membranes can be performed essentially according to the method previously described by Bruns et al., 1980, Proc. Natl. Acad. Sci. 77, 5547-5551, which is incorporated herein by reference in its entirety. Displacement experiments can be performed in 0.25 ml of buffer containing 1 nM [³H]-DPCPX, 100 ul of diluted membranes of rat brain (100 μg of protein/assay) and at least 6-8 different concentrations of examined compounds. Non specific binding can be determined in the presence of 10 uM of CHA (N6 cyclohexyladenosine) and this is always ≦10% of the total binding. Incubation times are typically 120 min at 25° C.

Radioligand binding assays may comprise the following: Binding of [³H]-SCH 58261 (5-amino-7-(2-phenylethyl)-2-(2-furyl)-pyrazolo[4,3-e]-1,2,4-triazolo[1,5-c]pyrimidine) to rat striatal membranes (100 ug of protein/assay) can be performed according to methods described in Zocchi et al., 1996, J. Pharm. and Exper. Ther. 276:398-404, which is incorporated herein by reference in its entirety. In competition studies, at least 6-8 different concentrations of examined compounds should be used. Non specific binding can be determined in the presence of 50 uM of NECA (5′-(N-ethylcarboxamido)adenosine). Incubation time is typically 60 min at 25° C. Bound and free radioactivity can be separated by filtering the assay mixture through Whatman GF/B glass-fiber filters using a Brandel cell harvester (Gaithersburg, Md., USA). The incubation mixture can be diluted with 3 ml of ice-cold incubation buffer, rapidly vacuum filtered and the filter can be washed three times with 3 ml of incubation buffer. The filter bound radioactivity can be measured, for example, by liquid scintillation spectrometry. The protein concentration can be determined, for example, according to a Bio-Rad method (Bradford, 1976, Anal. Biochem. 72:248, which is incorporated herein by reference in its entirety) with bovine albumin as reference standard.

An exemplary assay for human cloned A₃ Adenosine Receptor Binding Assay may comprise the following: Binding assays can be carried out according to methods described in Salvatore et al., 1993, Proc. Natl. Acad. Sci. 90:10365-10369; which is incorporated herein by reference in its entirety. In saturation studies, an aliquot of membranes (8 mg protein/ml) from HEK-293 cells transfected with the human recombinant A₃ adenosine receptor (Research Biochemical International, Natick, Mass., USA) can be incubated with 10-12 different concentrations of [¹²⁵I]AB-MECA ranging from 0.1 to 5 nM. Competition experiments can be carried out in duplicate in a final volume of 100 ul in test tubes containing 0.3 nM [¹²⁵I]AB-MECA, 50 mM Tris HCl buffer, 10 mM MgCl₂, pH 7.4 and 20 ul of diluted membranes (12.4 mg protein/ml) and at least 6-8 different concentrations of examined ligands. Incubation time was 60 min at 37° C., according to the results of previous time-course experiments. Bound and free radioactivity were separated by filtering the assay mixture through Whatman GF/B glass-fiber filters using a Brandel cell harvester. Non-specific binding was defined as binding in the presence of 50 uM R-PIA and was about 30% of total binding. The incubation mixture was diluted with 3 ml of ice-cold incubation buffer, rapidly vacuum filtered and the filter was washed three times with 3 ml of incubation buffer. The filter bound radioactivity was counted in a Beckman gamma 5500B gamma counter. The protein concentration can be determined according to a Bio-Rad method with bovine albumin as reference standard.

Data Analysis may be carried out as follows: Inhibitory binding constant, Ki, values can be calculated from those of IC₅₀ according to the Cheng & Prusoff equation (Cheng and Prusoff, 1973, Biochem. Pharmacol. 22:3099-3108), Ki=IC₅₀/(1+[C*]/K_{D}*), where [C*] is the concentration of the radioligand and K_{D}* its dissociation constant. A weighted non linear least-squares curve fitting program LIGAND (Munson and Rodbard, 1990, Anal. Biochem. 107:220-239) can be used for computer analysis of saturation and inhibition experiments. Data are typically expressed as geometric mean, with 95% or 99% confidence limits in parentheses.

In some embodiments, the efficacy of the compounds can be tested in animal models. For example, the efficacy of the adenosine A₃ antagonists alone or in combination with conventional anti-tumor agents such as cytotoxic/anti-neoplastic agents and anti-angiogenic agents can be compared to the conventional agents alone. Typically, a tumor of a given size is present in a rat or mouse. The mouse is treated with the agent and the size of the tumor is measured over time. The mean survival time of the animals can also be measured. The adenosine A₃ antagonists which are used must actively bind the adenosine A₃ receptor in the animal which is to be tested. Xenografts can be implanted into the animal to test the ability of species specific cytotoxic compounds. However, the effect of the anti-angiogenic therapy depends on how well the anti-angiogenetic compounds work on inhibiting the specific animal's vasculation.

Assay methods that have demonstrated the efficacy of endostatin and angiostatin in suppressing tumor growth can be applied to A₁, A₂ₐ, and A₃ antagonists. For example, Lewis lung carcinoma, T241 fibrosarcoma or B16F10 melanoma can be grafted to mice (Boehm et al., 1977, Nature, 390:404-407; which is incorporated herein by reference in its entirety). Other tumors, such as PC-3 human prostate carcinoma, the CCL188 human colon carcinoma and the UBC urinary bladder carcinoma have been shown to release angiogenic agents (Chen et al., 1996, Cancer Res., 55:4230-4322; which is incorporated herein by reference in its entirety). these tumors can also be grafted in mice for the evaluation of anti-tumor activities of adenosine antagonists. Adenosine antagonists can be administered to mice by the methods disclosed herein. This therapy can be used alone or in conjunction with anti-angiogenic therapy or conventional chemotherapy.

6.5.3 Methods for Determining and Measuring HIF-1 Alpha Levels

The invention encompasses methods to assess quantitative and qualitative aspects of HIF-1α expression. In one example, the increased expression of an HIF-1α gene or gene product indicates a predisposition for the development of cancer or a disease or disorder associated with enhanced HIF-1α expression. Alternatively, enhanced expression levels of an HIF-1α gene or gene product can indicate the presence of cancer in a subject or the risk of metastasis of said cancer in said subject. Techniques well known in the art, e.g., quantitative or semi-quantitative RT PCR or Northern blot, can be used to measure expression levels of HIF-1α. Methods that describe both qualitative and quantitative aspects of HIF-1α gene or gene product expression are described in detail in the examples infra. The measurement of HIF-1α gene expression levels can include measuring naturally occurring HIF-1α transcripts and variants thereof as well as non-naturally occurring variants thereof, however for the diagnosis and/or prognosis of diseases or disorders in a subject the HIF-1α gene product is preferably a naturally occurring HIF-1α gene product or variant thereof. Thus, the invention relates to methods of measuring the expression of the HIF-1α gene or gene product in a subject.

Any method known in the art for detecting and/or quantitating an HIF-1α level may be used in the methods and kits of the invention, a number of which are exemplified herein. Particularly preferred are methods known in the art for detecting and/or quantitating an HIF-1α activity or an HIF-1α related activity, e.g., phosphorylation of downstream effector molecules in the HIF-1α pathway. In some embodiments, the invention encompasses measuring an HIF-1α activity or an HIF-1α related activity including but not limited to, measuring an activity of one or more downstream effectors of an HIF-1α signaling cascade. Measuring an HIF-1α activity or an HIF-1α related activity can be done using any of the methods disclosed herein or any standard method known to one skilled in the art.

In other embodiments, the invention encompasses quantitation of a nucleic acid encoding HIF-1α in a sample obtained from a subject using methods disclosed herein or any standard method known in the art.

In yet other embodiments, the invention encompasses quantitation of HIF-1α protein in a sample obtained from a subject with a disease or disorder, e.g., cancer. Any method known in the art for the detection and quantitation of a HIF-1α protein is encompassed within the present invention.

6.5.3.1 Detection of Nucleic Acid Molecules

The methods and kits of the invention encompass detection and/or quantitation of a nucleic acid sequence encoding HIF-1α in a sample obtained from a subject. In certain embodiments, the invention provides methods for amplifying a specific HIF-1α nucleic acid sequence in a sample obtained from a subject with cancer, and detecting and/or quantitating the same. Nucleic acids encoding HIF-1α are well known in the art. See, for example, Wang et al., 1995, Proc. Natl. Acad. Sci. USA, 92: 5510-4; and WO 96/39426 each of which is incorporated herein by reference in their entireties.

The methods and kits of the invention may use any nucleic acid amplification or detection method known to one skilled in the art, such as those described in U.S. Pat. Nos. 5,525,462; 6,528,632; 6,344,317; 6,114,117; 6,127,120; 6,448,001; all of which are incorporated herein by reference in their entirety.

In some embodiments, the nucleic acid encoding an HIF-1α is amplified by PCR amplification using methodologies known to one skilled in the art. One of skill in the art will recognize, however, that amplification of target sequences (i.e., nucleic acid sequences encoding HIF-1α) in a sample obtained from a subject with cancer can be accomplished by any known method, such as ligase chain reaction (LCR), QP-replicase amplification, transcription amplification, and self-sustained sequence replication, each of which provides sufficient amplification. The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; which is incorporated herein by reference in its entirety). Also see U.S. Pat. No. 4,683,202; which is incorporated herein by reference in its entirety. PCR reagents and protocols are also available from commercial vendors, such as Roche Molecular Systems.

The invention encompasses methods to determine quantitative and/or qualitative levels of expression of HIF-1α. Any technique known in the art for measuring the expression of an HIF-1α is within the scope of the invention, including but not limited, to quantitative and/or semi-quantitative RT PCR and Northern blot analysis.

In some embodiments, the invention encompasses detecting and/or quantitating an HIF-1α nucleic acid using fluorescence in situ hybridization (FISH) in a sample, preferably a tissue sample, obtained from a subject with cancer in accordance with the methods of the invention. FISH is a common methodology used in the art, especially in the detection of specific chromosomal aberrations in tumor cells, for example, to aid in diagnosis and tumor staging. As applied in the methods of the invention, it can also be used as a method for detection and/or quantitation of an HIF-1α nucleic acid. For a review of FISH methodology, see, e.g., Weier et al., 2002, Expert Rev. Mol. Diagn. 2(2): 109-119; Trask et al., 1991, Trends Genet. 7(5): 149-154; and Tkachuk et al., 1991, Genet. Anal. Tech. Appl. 8: 676-74; all of which are incorporated herein by reference in their entirety.

The invention encompasses measuring naturally occurring HIF-1α transcripts and variants thereof as well as non-naturally occurring variants thereof. For the prognosis of cancer in a subject using the methods of the invention, the HIF-1α transcript is preferably a naturally occurring HIF-1α transcript.

In some embodiments, the invention relates to methods of prognosis of a cancer in a subject by measuring the expression of an HIF-1α transcript in a subject. For example, the increased level of mRNA encoding an HIF-1α, as compared to a standard, e.g., a non-cancerous sample, would indicate the increased risk of developing cancer in said subject. In another embodiment, the increased level of mRNA encoding an HIF-1α as compared to a standard would indicate the risk of metastasis of the cancer in said subject or the likelihood of a poor prognosis in said subject.

In one embodiment, the invention encompasses isolating RNA from a sample obtained from a subject with cancer, and testing the RNA utilizing hybridization or PCR techniques as described above for determining the level of an HIF-1α. In another embodiment, the invention encompasses synthesizing cDNA from the isolated RNA by reverse transcription. All or part of the resulting cDNA is then used as the template for a nucleic acid amplification reaction, such as a PCR or the like. The nucleic acid reagents used as synthesis initiation reagents (e.g., primers) in the reverse transcription and nucleic acid amplification steps of this method are chosen from among the HIF-1α nucleic acid reagents described below. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

In alternative embodiments, standard Northern analysis techniques known to one skilled in the art can be performed on a sample obtained from a subject with cancer. The preferred length of a probe used in Northern analysis is 9-50 nucleotides. Utilizing such techniques, quantitative as well as size related differences among HIF-1α transcripts can also be detected.

In alternative embodiments, the invention encompasses gene expression assays in situ, i.e., directly upon tissue sections (fixed and/or frozen) of patient tissue obtained from biopsies or resections, such that no nucleic acid purification is necessary. Nucleic acid reagents such as those described below may be used as probes and/or primers for such in situ procedures (see, e.g., Nuovo, G. J., 1992, PCR In Situ Hybridization: Protocols And Applications, Raven Press, NY, which is incorporated herein by reference in its entirety).

The target HIF-1α nucleic acids of the invention can also be detected using other standard techniques well known to those of skill in the art. Although the detection step is typically preceded by an amplification step, amplification is not necessarily required in the methods of the invention. For instance, the HIF-1α nucleic acids can be identified by size fractionation (e.g., gel electrophoresis). The presence of different or additional bands in the sample as compared to the control is an indication of the presence of target nucleic acids of the invention. Alternatively, the target HIF-1α nucleic acids can be identified by sequencing according to well known techniques. In alternative embodiments, oligonucleotide probes specific to the target HIF-1α nucleic acids can be used to detect the presence of specific fragments.

Sequence-specific probe hybridization is a well known method of detecting desired nucleic acids in a sample comprising a biological fluid or tissue sample and is within the scope of the present invention. Briefly, under sufficiently stringent hybridization conditions, the probes hybridize specifically only to substantially complementary sequences. The stringency of the hybridization conditions can be relaxed to tolerate varying amounts of sequence mismatch. If the target is first amplified, detection of the amplified product utilizes this sequence-specific hybridization to insure detection of only the correct amplified target, thereby decreasing the chance of a false positive caused by the presence of homologous sequences from related organisms or other contaminating sequences.

A number of hybridization formats well known in the art, including but not limited to solution phase, solid phase, mixed phase, or in situ hybridization assays are encompassed within the nucleic acid detection methods of the invention. In solution (or liquid) phase hybridizations, both the target nucleic acid and the probe or primer are free to interact in the reaction mixture. In solid phase hybridization assays, either the target or probes are linked to a solid support where they are available for hybridization with complementary nucleic acids in solution. Exemplary solid phase formats include Southern hybridizations, dot blots, and the like. The following articles provide an overview of the various hybridization assay formats, all of which are incorporated herein by reference in their entirety: Singer et al., 1986 Biotechniques 4: 230; Haase et al., 1984, Methods in Virology, Vol. VII, pp. 189-226; Wilkinson, In Situ Hybridization, D. G. Wilkinson ed., IRL Press, Oxford University Press, Oxford; and Nucleic Acid Hybridization: A Practical Approach, Hames, B. D. and Higgins, S. J., eds., IRL Press (1987).

The invention encompasses homogenous based hybridization assays as well as heterogeneous based assays for detection and/or quantitation of HIF-1α nucleic acid sequences in accordance with the methods of the invention. Heterogeneous based assays depend on the ability to separate hybridized from non-hybridized nucleic acids. One such assay involves immobilization of either the target or probe nucleic acid on a solid support so that non-hybridized nucleic acids which remain in the liquid phase can be easily separated after completion of the hybridization reaction (see, e.g., Southern, 1975, J. Mol. Biol. 98: 503-517; which is incorporated herein by reference in its entirety). In comparison, homogeneous assays depend on other means for distinguishing between hybridized and non-hybridized nucleic acids. Because homogeneous assays do not require a separation step, they are generally considered to be more desirable. One such homogeneous assay relies on the use of a label attached to a probe nucleic acid that is only capable of generating a signal when the target is hybridized to the probe (see, e.g., Nelson, et al., 1992, Nonisotopic DNA Probe Techniques, Academic Press, New York, N.Y., pages 274-310; which is incorporated herein by reference in its entirety).

The invention encompasses any method known in the art for enhancing the sensitivity of the detectable signal in such assays, including but not limited to the use of cyclic probe technology (Bakkaoui et al., 1996, BioTechniques 20: 240-8, which is incorporated herein by reference in its entirety); and the use of branched probes (Urdea et al., 1993, Clin. Chem. 39: 725-6; which is incorporated herein by reference in its entirety).

The hybridization complexes are detected according to well known techniques in the art. Nucleic acid probes capable of specifically hybridizing to a target can be labeled by any one of several methods typically used to detect the presence of hybridized nucleic acids. One common method of detection is the use of autoradiography, using probes labeled with ³H, ¹²⁵I, ³⁵S, ¹⁴C, or ³²P, or the like. The choice of radioactive isotope depends on research preferences due to ease of synthesis, stability, and half lives of the selected isotopes. Other labels include compounds (e.g., biotin and digoxigenin), that bind to anti-ligands or antibodies labeled with fluorophores, chemiluminescent agents, or enzymes. Alternatively, probes can be conjugated directly to labels such as fluorophores, chemiluminescent agents or enzymes. The choice of label depends on sensitivity required, ease of conjugation with the probe, stability requirements, and available instrumentation.

The probes and primers of the invention can be synthesized and labeled using techniques known to one skilled in the art. Oligonucleotides for use as probes and primers may be chemically synthesized according to the solid phase phosphoramidite triester method described by Beaucage, S. L. and Caruthers, M. H., 1981, Tetrahedron Lett. 22(20): 1859-1862, using an automated synthesizer, as described in Needham-VanDevanter, D. R., et al. 1984, Nucleic Acids Res. 12: 6159-6168. Purification of oligonucleotides can be by either native acrylamide gel electrophoresis or by anion-exchange HPLC, as described in Pearson, J. D. and Regnier, F. E., 1983, J. Chrom. 255:137-149. All of the references cited supra are incorporated herein by reference in their entirety.

6.5.4 Detection of Proteins

The methods and kits of the invention encompass detection and/or quantitation of HIF-1α in a sample obtained from a subject. Any method known to one skilled in the art for the detection and quantitation of an HIF-1α protein is encompassed within the present invention. HIF-1α protein sequences useful in the methods and kits of the invention are well known in the art. See, for example, Wang et al., 1995, Proc. Natl. Acad. Sci. USA, 92: 5510-4; Merighi et al., HIF-1α Expression in Cancer Cells in up-regulated by adeerrozine A₃ receptors, paper submitted to Nature, 2004; and WO 96/39426 each of which is incorporated herein by reference in its entireties.

HIF-1α proteins and anti-HIF-1α antibodies and immunospecific fragments thereof are suitable in the assays of the invention. Detection and quantitation of an HIF-1α gene product encompasses the detection of proteins exemplified herein. Detection of elevated levels of an HIF-1α gene product in a sample obtained from a subject in accordance with the methods of the invention is generally compared to a standard sample.

In some embodiments, antibodies directed against naturally occurring HIF-1α proteins may be used in the methods of the invention. The invention encompasses the use of any standard immunoassay method known to one skilled in the art, including but not limited to Western blot, ELISA, and FACS.

In one embodiment, the invention encompasses use of an immunoassay comprising contacting a sample from a subject with an anti-HIF-1α antibody or an immunospecific fragment thereof under conditions such that immunospecific binding to the HIF-1α receptor in the sample can occur, thereby forming an immune complex, and detecting and/or measuring the amount of complex formed. In a specific embodiment, an antibody to an HIF-1α is used to assay a sample for the presence of the HIF-1α, wherein an increased level of the HIF-1α is detected relative to a standard sample.

In some embodiments, the biological sample may be brought in contact with and immobilized onto a solid phase support or a carrier such as nitrocellulose or other solid support capable of immobilizing cells, cell particles or soluble proteins. The support can be washed with suitable buffers followed by treatment with the antibody that selectively or specifically binds to an HIF-1α protein. The solid phase support can then be washed with buffer to remove unbound antibody. The amount of antibody bound to the solid support can then be detected by conventional means.

“Solid phase support or carrier” as used herein refers to any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

In some embodiments, the anti-HIF-1α antibody or an immunospecific fragment thereof can be detectably labeled by linking the same to an enzyme and using the labeled antibody in an enzyme immunoassay (EIA) (Voller, A., “The Enzyme Linked Immunosorbent Assay (ELISA)”, 1978, Diagnostic Horizons 2:1, Microbiological Associates Quarterly Publication, Walkersville, Md.; Voller, A. et al., 1978, J. Clin. Pathol. 31:507-520; Butler, J. E., 1981, Meth. Enzymol. 73:482; Maggio, E. ed., 1980, Enzyme Immunoassay, CRC Press, Boca Raton, Fla.; Ishikawa, E. et al., eds., 1981, Enzyme Immunoassay, Kgaku Shoin, Tokyo, all of which are incorporated herein by reference in their entirety). The enzyme bound to the antibody will react with an appropriate substrate, preferably a chromogenic substrate, in such a manner as to produce a chemical moiety which can be detected, for example, by spectrophotometric, fluorimetric or visual means. Enzymes that can be used to detectably label the antibody include but are not limited to malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase, among others. The detection can be accomplished by colorimetric methods that employ a chromogenic substrate for the enzyme. Detection can also be accomplished by visual comparison of the extent of enzymatic reaction of a substrate in comparison with similarly prepared standards.

Detection can also be accomplished using any other method known to one skilled in the art. For example, by radioactively labeling the antibodies or antibody fragments, it is possible to detect HIF-1α protein through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter, or by autoradiography.

In other embodiments, the invention encompasses labeling the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. In yet other embodiments, the antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The invention further encompasses detectably labeling the antibody by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds for purposes of labeling include, e.g., luciferin, luciferase and aequorin.

The invention also encompasses methods for indirect detection of HIF-1α. In a specific embodiment, the invention encompasses use of an immunoassay comprising contacting a sample derived from a subject with cancer with an anti-HIF-1α antibody (primary antibody) or an immunospecific fragment thereof under conditions such that immunospecific binding to the HIF-1α protein in the sample can occur, thereby forming an immune complex, adding a secondary antibody that is labeled under conditions such that immunospecific binding to the primary antibody occurs and detecting and/or quantitating the amount of complex formed indirectly.

Anti-HIF-1α antibodies or immunospecific fragments thereof may be used quantitatively or qualitatively to detect an HIF-1α in a sample. In some embodiments, when the sample is a tissue, the anti-HIF-1α antibodies or immunospecific fragments thereof may be used histologically, e.g., immunofluorescence or microscopic studies, using common techniques known to one skilled in the art, for in situ detection of an HIF-1α receptor. In situ detection may be accomplished by preparing a histological specimen from a subject, such as a paraffin embedded section of tissue, e.g., breast tissues, and applying thereto a labeled antibody of the present invention. The antibody (or fragment) is preferably applied by overlaying the labeled antibody (or fragment) onto the biological sample. Through the use of such a procedure, it is possible to determine not only the presence of an HIF-1α protein but also its distribution in the examined tissue. Using the methods of the present invention, those of ordinary skill will readily perceive that any of a wide variety of histological methods (such as staining procedures) can be modified in order to achieve such in situ detection.

6.6 Nucleic Acids Encoding HIF-1 Alpha

The methods of the invention may use any nucleic acid encoding HIF-1α, an analog, fragment or derivative thereof, as a proxy for determining a HIF-1α level. A nucleic acid is intended to include DNA molecules (e.g., cDNA, genomic DNA), RNA molecules (e.g., hnRNA, pre-mRNA, mRNA) and DNA or RNA analogs (e.g., peptide nucleic acids) generated using techniques known to one skilled in the art. The nucleic acid measured as a proxy for a HIF-1α level can be single-stranded or double stranded.

For example, but not by way of limitation, nucleotide sequences for use in the methods and kits of the invention may include all or a portion of any of the following: nucleotide sequences disclosed in U.S. Pat. No. 6,455,674 (issued to Einat et al); U.S. Pat. No. 6,652,799 (issued to Semenza); U.S. Pat. No. 6,222,018(issued to Semenza); Wang et al., 1995 PNAS USA, 92: 5510-4; and WO 96/39426. The invention encompasses all or a portion of the nucleotide sequence of human HIF-1α with GENBANK Accession Nos. NM_{—}001530 and NM_{—}181054. All nucleotide sequences of the references cited supra are incorporated herein by reference in their entirety.

Generally, any HIF-1α nucleic acid known in the art may be useful in the methods and kits of the invention. Such nucleic acids generally encode at least a portion of HIF-1α, or have a sequence that hybridizes to a HIF-1α, -encoding nucleic acid under hybridizing conditions, as described herein.

In one embodiment, the methods of the invention may use a coding sequence or a 5′ or 3′ untranslated region of a nucleic acid encoding HIF-1α or a fragment thereof as a probe, including naturally occurring and non-naturally occurring variants. A non-naturally occurring variant is one that is engineered by man (e.g., a peptide nucleic acid probe). In the methods of the invention wherein HIF-1α, or an mRNA encoding HIF-1α, in a sample from a subject is detected or measured, naturally occurring gene products are detected, including but not limited to wild-type gene products as well as mutants, allelic variants, splice variants, polymorphic variants, etc. In general, variants will be highly homologous to the wild-type gene product encoding HIF-1α, e.g., having at least 90%, 95%, 98% or 99% amino acid sequence identity (as determined by standard algorithms known in the art, see, e.g., Altschul, 1990 Proc. Natl. Acad. Sci. U.S.A. 87: 2264-2268; Altschul, 1993, Proc. Natl. Acad. Sci. U.S.A. 90: 5873-5877; Altschul etal., 1990 J. Mol. Biol. 215: 403-410).

HIF-1α variants to be used as probes may be encoded by a nucleic acid which is hybridizable under stringent conditions to a nucleic acid encoding HIF-1α. Nucleic acid hybridization methods are well known in the art (see, e.g., Sambrook et al., 2001 Molecular Cloning, A Laboratory Manual, 3^{rd} ed., Cold Spring Harbor Laboratory Press, Cold Spring Harbor, N.Y.; Ausubel et al., eds., 1994-1997, in the Current Protocols in Molecular Biology: Series of laboratory technique manuals, John Wiley and Sons, Inc.; Shilo and Weinberg, 1981, Proc. Natl. Acad. Sci. U.S.A. 78, 6789-92; Dyson, 1991 Essential Molecular Biology: A Practical Approach, vol. 2, T. A. Brown, ed., 111-156, Press at Oxford University Press, Oxford, UK). The term “stringent conditions” refers to the ability of a first polynucleotide molecule to hybridize, and remain bound to a second filter-bound polynucleotide molecule in 0.5 M NaHPO₄, 7% sodium dodecyl sulfate (SDS), 1 mM EDTA at 65° C., followed by washing in 0.2×SSC/0.1% SDS at 42° C. (see Ausubel et al. (eds.), 1989, Current Protocols in Molecular Biology, Vol. 1, Green Publishing Associates, Inc., and John Wiley & Sons, Inc., New York, at p. 2.10.3). In specific embodiments, the variants being detected or measured comprise (or, if nucleic acids, encode) not more than 1, 2, 3, 4, 5, 10, 15 or 20 point mutations (substitutions) relative the wild-type sequence.

An isolated nucleic acid probe encoding HIF-1α or a portion thereof, can be obtained by any method known in the art, e.g., from a deposited plasmid, by PCR amplification using synthetic primers hybridizable to the 3′ and 5′ ends of the sequence, and/or by cloning from a cDNA or genomic library using standard screening techniques, or by polynucleotide synthesis. Use of such probes for detection and quantitation of specific sequences is well known in the art. See e.g., Erlich, ed., 1989, PCR Technology Principles and Applications for DNA Amplification, Macmillan Publishers Ltd., England; Sambrook et al, Molecular Cloning: A Laboratory Manual, 3^{rd} ed., Cold Spring Harbor Laboratory Press, Cold Spring Harbor, N.Y., 2001.

In some embodiments, the methods of the invention may use a gene coding sequence, e.g., cDNA, of HIF-1α, which preferably hybridizes under stringent conditions as described above to at least about 6, preferably about 12, most preferably about 18 or more consecutive nucleotides of the gene coding sequence of HIF-1α protein, useful for the detection of an HIF-1α protein

Using all or a portion of a nucleic acid sequence encoding HIF-1α protein, such as those exemplified herein as a hybridization probe, full length nucleic acid molecules encoding HIF-1α protein can be quantitated using standard hybridization techniques (see, e.g., Sambrook et al., Molecular Cloning: A Laboratory Manual, 3^{rd} ed., Cold Spring Harbor Laboratory, Cold Spring Harbor Laboratory Press, Cold Spring Harbor, N.Y., 2001) for use in the methods of the invention, i.e., as a proxy for HIF-1α level.

The HIF-1α sequences used in the methods of the invention are preferably human sequences. However, homologs of the human HIF-1α isolated from other animals can also be used in the methods of the invention as a proxy for HIF-1α level, particularly where the subject is a non-human animal. Thus, the invention also includes the use of HIF-1α homologs identified from non-human animals such as non-human primates, rats, mice, farm animals including but not limited to cattle, horses, goats, sheep, pigs, etc., household pets including but not limited to cats, dogs, etc., in the methods of the invention.

The methods of the invention may use fragments of any of the nucleic acids disclosed herein in any of the methods of the invention. A fragment preferably comprises at least 10, 20, 50, 100, or 200 contiguous nucleotides of a sequence described herein.

Standard recombinant DNA techniques known in the art may be used to provide a HIF-1α protein or a nucleic acid encoding an HIF-1α protein, or a fragment thereof, for use in the methods and kits of the invention. In some embodiments, in order to provide a HIF-1α or nucleic acid as a standard, the corresponding nucleotide sequence encoding HIF-1α protein of interest can be cloned. For a review of PCR technology and cloning strategies which may be used in accordance with the invention, see, e.g., PCR Primer, 1995, Dieffenbach et al., ed., Cold Spring Harbor Laboratory Press; Sambrook et al., 2001, supra which are incorporated herein by reference in their entireties.

6.7 HIF-1 Alpha Proteins

The present invention provides for the use of HIF-1α proteins, or fragments thereof, for the generation of antibodies for methods of the invention. HIF-1α polypeptides and fragments can also be used as protein abundance or activity standards in the methods of the invention.

For example, but not by way of limitation, the invention encompasses amino acid sequences of HIF-1α as dislcosed in U.S. Pat. Nos. 6,455,674 (issued to Einat et al); U.S. Pat. No. 6,652,799 (issued to Semenza); U.S. Pat. No. 6,222,018(issued to Semenza); Wang et al., 1995 PNAS USA, 92: 5510-4; 6,436,654 (issued to Berkenstam); WO 96/39426. The amino acid sequences cited in the above-identified references are incorporated herein by reference in their entirety. The invention encompasses the amino acid sequences of human HIF-1α with GENBANK Accession No.s NP_{—}001521 and NP_{—}851397 each of which is incorporated herein by reference in its entirety.

In some embodiments, the HIF-1α protein comprises an amino acid sequence that exhibits at least about 65%, 70%, 75%, 80%, 85%, 90%, 95%, or 99% sequence similarity to the amino acid sequence of any of HIF-1α proteins known in the art. Algorithms for determining percent identity between two protein sequences are well known in the art, see, e.g., Altschul, 1990 Proc. Natl. Acad. Sci. U.S.A. 87: 2264-2268; Altschul, 1993, Proc. Natl. Acad. Sci. U.S.A. 90: 5873-5877; Altschul et al., 1990 J. Mol. Biol. 215: 403-410; each of which is incorporated herein by reference.

In a specific embodiment, proteins are provided consisting of or comprising a fragment of HIF-1α protein consisting of at least ten contiguous amino acids. In another embodiment, the fragment consists of or comprises at least 20, 30, 40, or 50 contiguous amino acids from a HIF-1α protein for use, for example, in raising antibodies. Such fragments can also be useful, for example, as standards or controls in the methods and kits of the invention.

A variety of host-expression vector systems may be utilized to express HIF-1α proteins or fragments for use in the methods of the invention. Such host-expression systems are well known and provide the necessary means by which a protein of interest may be produced and subsequently purified. Examples of host-expression vector systems that may be used in accordance with the invention are: bacterial cells (e.g., E. coli, B. subtilis) transformed with recombinant bacteriophage DNA, plasmid DNA or cosmid DNA expression vectors containing a HIF-1α nucleic acid coding sequence, yeast cells (e.g., Saccharomyces, Pichia) transformed with a recombinant yeast expression vector containing the HIF-1α coding sequence; insect cells infected with a recombinant virus expression vector (e.g., baculovirus) containing the HIF-1α coding sequence; plant cells infected with a recombinant virus expression vector (e.g., cauliflower mosaic virus, CaMV; tobacco mosaic virus, TMV) or transformed with a recombinant plasmid expression vector (e.g., Ti plasmid) containing the HIF-1α coding sequence; or mammalian cells (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the HIF-1α being expressed. For example, when a large quantity of such a protein is to be produced for raising antibodies, vectors that direct the expression of high levels of protein products that are readily purified may be desirable. Such vectors include but are not limited to the E. coli expression vector pUR278 (Ruther et al., 1983, EMBO J. 2:1791), in which the HIF-1α coding sequence can be ligated into the vector in-frame with the lac Z coding region so that a fusion protein is produced; pIN vectors (Inouye & Inouye, 1985, Nucleic Acids Res. 13:3101; Van Heeke & Schuster, 1989, J. Biol. Chem. 264:5503); and the like. pGEX vectors can also be used to express foreign polypeptides as fusion proteins with glutathione S-transferase (GST). In general, such fusion proteins are soluble and can easily be purified from lysed cells by adsorption and binding to a column comprising of glutathione-agarose beads followed by elution in the presence of free glutathione. The pGEX vectors are designed to include, e.g., thrombin or factor Xa protease cleavage sites so that the cloned target gene product can be released from the GST moiety.

In an insect system, Autographa californica nuclear polyhedrosis virus (AcNPV) can be used as a vector to express foreign genes. The virus grows in Spodoptera frugiperda cells. The HIF-1α coding sequence can be cloned individually into non-essential regions (for example the polyhedrin gene) of the virus and placed under control of an AcNPV promoter (for example the polyhedrin promoter). Successful insertion of a HIF-1α coding sequence will result in inactivation of the polyhedrin gene and production of non-occluded recombinant virus (i.e., virus lacking the proteinaceous coat coded for by the polyhedrin gene). These recombinant viruses can be used to infect Spodoptera frugiperda cells in which the inserted gene is expressed (e.g., see Smith et al., 1983, J. Virol. 46:584; Smith, U.S. Pat. No. 4,215,051).

In mammalian host cells, a number of viral-based expression systems can be utilized. In cases where an adenovirus is used as an expression vector, the HIF-1α coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing HIF-1α in infected hosts (see, e.g., Logan & Shenk, 1984, Proc. Natl. Acad. Sci. USA 81:3655). Specific initiation signals may also be required for efficient translation of inserted HIF-1α coding sequences. These signals include the ATG initiation codon and adjacent sequences. In cases where an entire HIF-1α gene, including its own initiation codon and adjacent sequences, is inserted into the appropriate expression vector, no additional translational control signals may be needed. However, in cases where only a portion of the HIF-1α coding sequence is inserted, exogenous translational control signals, including, if necessary, the ATG initiation codon, must be provided. These exogenous translational control signals and initiation codons can be of a variety of origins, both natural and synthetic. Furthermore, the initiation codon must be in phase with the reading frame of the desired coding sequence to ensure correct translation of the entire insert. The efficiency of expression may be enhanced by the inclusion of appropriate transcription enhancer elements, transcription terminators, etc. (see Bittner et al., 1987, Methods in Enzymol. 153: 516).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include but are not limited to CHO, VERO, BHK, HeLa, COS, MDCK, 293, 3T3, WI38, and in particular, breast cancer cell lines such as, for example, BT483, Hs578T, HTB26, BT20 and T47D, and normal mammary gland cell lines such as, for example, CRL7030 and Hs578Bst.

For long-term, high-yield production of recombinant proteins, stable expression is preferred. For example, cell lines that stably express the HIF-1α gene product can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker.

Following introduction of the foreign DNA, engineered cells can be allowed to grow for 1-2 days in an enriched media, and then can be switched to a selective media. A selectable marker in a recombinant construct, such as a plasmid, can confer resistance to the selective media, allow cells to stably integrate the plasmid into their chromosomes, and grow to form foci which, in turn, can be cloned and expanded into cell lines. This method can advantageously be used to engineer cell lines that stably express the HIF-1α gene product. Such engineered cell lines can be particularly useful in screening and evaluating compounds that affect the endogenous activity of the HIF-1α gene product.

A number of selection systems including but not limited to the herpes simplex virus thymidine kinase (Wigler et al., 1977, Cell 11: 223), hypoxanthine-guanine phosphoribosyltransferase (Szybalska & Szybalski, 1962, Proc. Natl. Acad. Sci. USA 48: 2026), and adenine phosphoribosyltransferase (Lowy et al., 1980, Cell 22: 817) genes can be employed in tk⁻, hgprt⁻ or aprt⁻ cells, respectively. Also, anti-metabolite resistance can be used as the basis of selection for the following genes: dhfr, which confers resistance to methotrexate (Wigler et al., 1980, Proc Natl. Acad. Sci. USA 77: 3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30: 147).

6.8 Antibodies to HIF-1 Alpha

The methods and kits of the invention encompass use of anti-HIF-1α antibodies or fragments thereof that specifically recognize one or more epitopes of a HIF-1α protein. Accordingly, any HIF-1α protein, derivative, or fragment can be used as an immunogen to generate antibodies that immunospecifally bind a HIF-1α protein. Such antibodies and fragments can be used in the detection and quantitation of a HIF-1α in a sample to carry out any of the methods of the invention as disclosed herein.

Such antibodies can include but are not limited to polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above. In a specific embodiment, antibodies to human HIF-1α protein are used.

Described herein are general methods for the production of antibodies or immunospecific fragments thereof. Any of such antibodies or fragments can be produced by standard immunological methods or by recombinant expression of nucleic acid molecules encoding the antibody or an immunospecific fragment thereof in an appropriate host organism.

For the production of antibodies against HIF-1α, any of various host animals can be immunized by injection with a HIF-1α gene product, or a portion thereof. Such host animals can include but are not limited to rabbits, mice, and rats. Various adjuvants can be used to increase the immunological response depending on the host species, including but not limited to Freund's (complete or incomplete), mineral gels such as aluminum hydroxide, surface active substances such as lysolecithin, pluronic polyols, polyanions, peptides, oil emulsions, keyhole limpet hemocyanin, dinitrophenol or potentially useful human adjuvants such as BCG (bacille Calmette-Guerin) and Corynebacterium parvum.

Anti-HIF-1α monoclonal antibodies are preferred for use in the methods and kits of the invention. Monoclonal antibodies can be obtained by any technique that provides for the production of antibody molecules by continuous cell lines in culture. These include but are not limited to the hybridoma technique of Kohler and Milstein, (1975, Nature 256: 495; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026), and the EBV-hybridoma technique (Cole et al., 1985, Monoclonal Antibodies And Cancer Therapy, Alan R. Liss, Inc., pp. 77). Such antibodies can be of any immunoglobulin class, including IgG, IgM, IgE, IgA, IgD, and any subclass thereof. The hybridoma producing the mAb of this invention can be cultivated in vitro or in vivo.

Techniques developed for the production of “chimeric antibodies” (Morrison et al., 1984, Proc. Natl. Acad. Sci. 81, 6851-6855; Neuberger et al., 1984, Nature 312, 604-608; Takeda et al., 1985, Nature 314, 452-454) by splicing the genes from a mouse antibody molecule of appropriate antigen specificity together with genes from a human antibody molecule of appropriate biological activity can be used in preparing antibodies useful in the present invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397). The invention thus contemplates chimeric antibodies that are specific or selective for a HIF-1α protein. While often designed to be therapeutic, such chimeric antibodies can be useful to quantitate a HIF-1α level according to the methods of the invention.

Further, humanized antibodies can be used in the methods and kits of the invention. Briefly, humanized antibodies are antibody molecules from non-human species having one or more hypervariable regions or complementarity determining regions (CDRs) from the non-human species and framework regions from a human immunoglobulin molecule. The extent of the framework region and Cars have been precisely defined (see, “Sequences of Proteins of Immunological Interest”, Kabat, E. et al., U.S. Department of Health and Human Services (1983)). Examples of techniques that have been developed for the production of humanized antibodies are known in the art and useful within the scope of the present invention. (See, e.g., Queen, U.S. Pat. No. 5,585,089 and Winter, U.S. Pat. No. 5,225,539). Humanized antibodies are typically developed as therapeutic agents. However, such antibodies can also be used in the methods and kits of the present invention, as they can be used to quantitate a HIF-1α level in accordance with the instant invention.

Phage display technology can be used to increase the affinity of an antibody to a HIF-1α gene product. This technique can be useful in obtaining higher affinity antibodies to an HIF-1α gene product, which could be used for the diagnosis and prognosis of a subject with cancer according to the present invention. The technology, referred to as affinity maturation, employs mutagenesis or CDR walking and re-selection using the HIF-1α gene product antigen to identify antibodies that bind with higher affinity to the antigen when compared with the initial or parental antibody (see, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones, each of which differs by a single amino acid alteration in a single CDR, and contain variants representing each possible amino acid substitution for each CDR residue. Mutants with increased binding affinity for the antigen can be screened by contacting the immobilized mutants with labeled antigen. Any screening method known in the art can be used to identify mutant antibodies having increased avidity to the antigen (e.g., ELISA) (see Wu et al., 1998, Proc Natl. Acad Sci. USA 95:6037; Yelton et al., 1995, J. Immunology 155:1994). CDR walking that randomizes the light chain may also be useful (see Schier et al., 1996, J. Mol. Bio. 263:551).

Alternatively, techniques described for the production of single chain antibodies (U.S. Pat. No. 4,946,778; Bird, 1988, Science 242:423; Huston et al., 1988, Proc. Natl. Acad. Sci. USA 85:5879; and Ward et al., 1989, Nature 334: 544) can be adapted to produce single chain antibodies against HIF-1α gene products. Single chain antibodies are formed by linking the heavy and light chain fragments of the Fv region via an amino acid bridge, resulting in a single chain polypeptide. Techniques for the assembly of functional Fv fragments in E. coli can also be used (Skerra et al., 1988, Science 242:1038).

Antibody fragments that recognize specific epitopes can be generated by known techniques. Such fragments can be used for quantitating a HIF-1α gene product according to any available method known in the art. For example, such fragments include but are not limited to: F(ab′)₂ fragments, which can be produced by pepsin digestion of the antibody molecule; and Fab fragments, which can be generated by reducing the disulfide bridges of the F(ab′)₂ fragments; Fab fragments, which can be generated by treating the antibody molecule with papain and a reducing agent; and Fv fragments. Alternatively, Fab expression libraries can be constructed (Huse et al., 1989, Science 246:1275-1281) to allow rapid and easy identification of monoclonal Fab fragments having the desired specificity.

A molecular clone of an antibody to an antigen of interest can be prepared by techniques known to one skilled in the art. Recombinant DNA methodology (see e.g., Maniatis et al., 1982, Molecular Cloning, A Laboratory Manual, Cold Spring Harbor Laboratory, Cold Spring Harbor, N.Y.) can be used to construct nucleic acid sequences that encode a monoclonal antibody molecule, or an immunospecific fragment thereof.

Antibody molecules can be purified by well-known techniques, e.g., immunoabsorption or immunoaffinity chromatography, chromatographic methods such as HPLC (high performance liquid chromatography), or a combination thereof.

In the production of antibodies, screening for the desired antibody can be accomplished by techniques known in the art, e.g., ELISA (enzyme-linked immunosorbent assay). For example, to select antibodies that recognize a specific domain of an HIF-1α, generated hybridomas can be assayed for a product that binds to a HIF-1α fragment containing such domain.

The foregoing antibodies can be used to quantify a HIF-1α protein, e.g., to measure levels thereof in appropriate samples, in the methods and kits of the invention.

The methods of antibody production employed herein include those described in Harlow and Lane (Harlow, E. and Lane, D., 1988, and later editions, Antibodies: A Laboratory Manual, Cold Spring Harbor Laboratory Press, Cold Spring Harbor, N.Y.), which is incorporated herein by reference in its entirety.

Any antibody directed to one or more epitopes of an HIF-1α can be used in the methods and kits of the invention. Commercially available HIF-1α antibodies can be used in accordance with the instant invention, for example those available from Novus Biologicals, Inc. (Littleton, Colo.); Affinity BioReagents, (Golden, Colo.).

6.9 Kits

The invention provides a pharmaceutical pack or kit comprising one or more containers filled with compounds of the invention. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. The present invention provides kits that can be used in the above methods. In one embodiment, a kit comprises one or more compounds of the invention. In another embodiment, a kit further comprises one or more other prophylactic or therapeutic agents useful for the treatment of cancer, in one or more containers. In another embodiment, a kit further comprises one or more anti-cancer agents that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

### 7. EXAMPLES

The following examples illustrate aspects of this invention but should not be construed as limitations. The symbols and conventions used in these examples are intended to be consistent with those used in the contemporary, international, chemical literature, for example, the Journal of the American Chemical Society (J.Am.Chem.Soc.) and Tetrahedron.

Materials and Methods—Chemicals and Reagents

Human A375, SKMES, HT29, Panc-1, Jurkat, HeL023 and NCTC2544 cells were obtained from American Tissue Culture Collection (ATCC). Tissue culture media and growth supplements were obtained from BioWhittaker. Unless otherwise noted, all other chemicals were purchased from Sigma. MRE 3046F20, 5-N-(4-methylphenyl-carbamoyl)amino-8-methyl-2(2-furyl)-pyrazolo-[4,3e]-1,2,4-triazolo[1,5-c]pyrimidine; IL-10 salt, 5-N-(4-diethylamino-phenyl-carbamoyl)amino-8-methyl-(2(2furyl)-pyrazolo-[4,3e]1,2,4-triazolo[1,5-c]pyrimidine); MRE 3008F20, 5-N-(4-methoxyphenyl-carbamoyl)amino-8-propyl-2(2furyl)-pyrazolo-[4,3e]1,2,4-triazolo[1,5-c]pyrimidine, MRE 3055F20, 5-N-(4-phenylcarbamoyl)amino-8-propyl-2(2furyl)-pyrazolo[4,3e]-1,2,4-triazolo-[1,5c]pyrimidine and MRE 3062F20, 5-N-(4-phenyl-carbumoyl)amino-8-butyl-2(2furyl)-pyrazolo-[4,3e]-1,2,4-triazolo[1,5-c]pyrimidine were synthesized by Prof. P. G. Baraldi, University of Ferrara, Italy. CGS15943, 5-amino-9-chloro-2-(furyl)1,2,4-triazolo[1,5-c]quinazoline and ZM 241385, 4-(2-[7-amino-2-(2-furyl)-[1,2,4]triazolo[2,3-a][1,3,5]triazin-5-ylamino]ethyl)phenol were obtained from RBI [Zeneca Pharmaceuticals, Cheshire, UK]. PD 98059, 2-Amino-3-methoxyflavone, (a selective inhibitor of MAP kinase (MEK)) and rhodamine 123 were obtained from Calbiochem. U0126 (an inhibitor of MEK-1 and MEK-2) and SB 203580 (an inhibitor of p38 MAP kinase) were from Promega. RNAse was purchased from Boehringer.

Cell Culture: For chemotherapeutic enhancement testing, the human melanoma A375, human lung carcinoma SKMES, colon carcinoma HT29, and pancreatic cancer Panc-1 cell lines were originally obtained from the ATCC (American Type Culture Collection) and have been maintained at PRC. Cell lines are maintained in RPMI-1640 medium supplemented with 100 units/ml penicillin G sodium, 100 μg/ml streptomycin sulfate, 0.25 μg/ml amphotericin B (fungizone), 2 mM glutamine, 10 mM HEPES, 25 μg/ml gentamycin, and 10% heat-inactivated fetal bovine serum. The cells are cultured in a T25 Falcon Tissue Culture flask in a humidified incubator at 37° C. with 5% CO₂-95% air. The cells are sub-cultured twice a week. The doubling times of A375, SKMES, HT29, and Panc-1 cultures are approximately 22, 45, 48, and 60 hr, respectively.

For multi-drug resistance testing, A375 and NCTC2544 cells are grown adherently and maintained in DMEM and EMEM medium, respectively, containing 10% fetal calf serum, penicillin (100 U/ml), streptomycin (100 μg/ml), L-glutamine (2 mM) at 37° C. in 5% CO₂/95% air. HeL023 and Jurkat were grown in RPMI-1640 medium, containing 10% fetal calf serum, penicillin (100 U/ml), streptomycin (100 μg/ml), L-glutamine (2 mM) at 37° C. in 5% CO₂/95% air. Cells are passaged two or three times weekly at a ratio between 1:5 and 1:10. Lymphocytes were isolated from buffy coats kindly provided by the Blood Bank of the University Hospital of Ferrara. Blood donated by healthy volunteers, after informed consent for research was obtained. Lymphocytes were isolated by density gradient centrifugation (Ficoll/Histopaque 1.077 g/ml). The cells are stimulated with purified phytohemoagglutinin (1 μg/ml) and expanded in RPMI medium added of interleukin 2 (20 Units/ml) and 10% fetal calf serum.

Colony Formation Assay: Exponentially growing A375 cells were seeded at 300 cells per well in six-well plates with 2 ml of fresh medium and treated with paclitaxel, vindesine and adenosine receptor agonists and antagonists dissolved in DMSO solution. Control plates received the same volume of DMSO alone. After 7 days of growth at 37° C. in humidified atmosphere containing 5% CO₂, the cells were fixed with absolute methanol for 5′ and stained with 1/10 Giemsa/phosphate-buffered saline (PBS) staining solution for 10 minutes. Staining solution was removed and colonies of greater than 30 cells were scored as survivors. For each treatment, six individual wells were scored.

Flow Cytometry Analysis: A375 and NCTC2544 adherent cells were trypsinized, mixed with floating cells, washed with PBS and permeabilised in 70% (vol/vol) ethanol/PBS solution at 4° C. for at least 24 hours. Jurkat, HeL023 and PBMC cells were centrifuged for 10 minutes at 1000×g. The cell pellet was then resuspended and permeabllised in 70% (vol/vol) ethanol/PBS solution at 4° C. for at least 24 hours. The cells were washed with PBS and the DNA was stained with a PBS solution, containing 20 μg/ml of propidium iodide and 100 μg/ml of RNAse, at room temperature for 30 minutes. Cells were analysed by FACScan (Becton-Dickinson) and the content of DNA was evaluated by the Cell-LISYS program (Becton-Dickinson). Cell distribution among cell cycle phases and apoptotic cells was evaluated as previously described (Secchiero et al., 2001). Briefly, the cell cycle distribution is shown as the percentage of cells containing 2n (G₁ phase), 4n (G₂ and M phases), 4n>x>2n. DNA amount (S phase) judged by propidium iodide staining. The apoptotic population is the percentage of cells with DNA content lower than 2n.

Rhodamine 123 Efflux Assay: 5×10⁵ cells from each subline were loaded with 50 ng/ml of rhodamine 123 for 30 minutes at 37 C in medium. The cells were washed and resuspended in dye-staining agent free medium for 3 hours at 37 C. to allow rhodamine 123 efflux. The cells were then washed twice and the fluorescence of intracellular rhodamine 123 were analysed with Flow cytometer (residual fluorescence FRES). The fluorescence was compared with loaded cells maintained at 4 C. to prevent drug export (maximal fluorescence FMAX). The cells were treated with adenosine receptor antagonists to evaluate the ability of adenosine receptors to interfere with P-gp drug efflux activity. Cells from each subline that were not being exposed to rhodamine 123 were used as negative controls.

Metabolic Inhibitors: Cells were treated for 30 minutes with metabolic inhibitors or with drug vehicle (DMSO) prior to being challenged with adenosine receptor antagonists and paclitaxel or vindesine. After 24 hours, cells were collected for flow cytometry analysis. PD 98059 was used at 20 uM as an inhibitor of MEK to prevent MEK-1 activation. U01 26 was used at 10 uM as inhibitor of MEK-1 and MEK-2 to prevent extracellular signal-regulated kinase ERK-1 and ERK-2 activation. SB 203580 was used at 1 uM as an inhibitor of p38MAP kinase (p38MAPK).

Statistical Analysis: All values in the figures and text are expressed as mean±.standard deviation (SD) of n observation (with n>3). Data sets were examined by analysis of variance (ANOVA) and Dunnett's test (when required). A P value less than 0.05 was considered statistically significant. Representative images obtained by FACscan are reported, with similar results having been obtained in at least three different experiments.

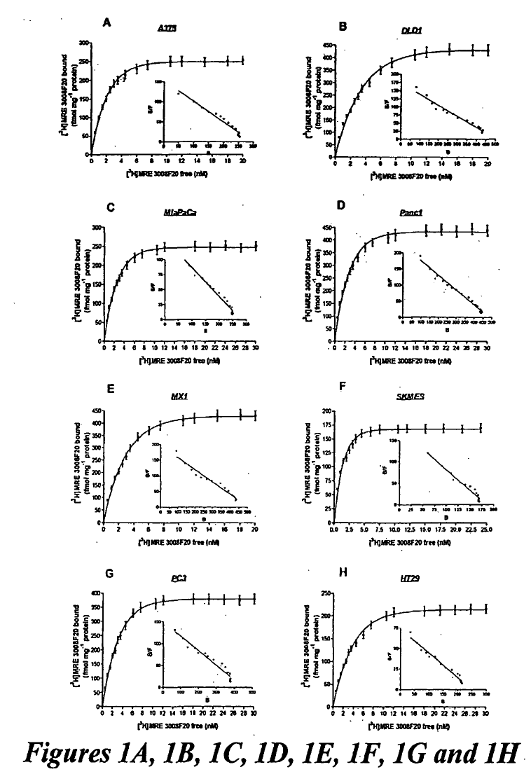

Inventors have verified the concentration of adenosine A₃ receptors in human cancer cells that is elevated in the cancerous tumors compared to non-cancerous tumors or normal tissue. Table 8 and FIG. 1 indicate elevated levels of adenosine A₃ receptors found by the inventors in human A375 (human melanoma), Panc-1 (human pancreatic carcinoma), MX-1 (human breast carcinoma), PC-1 (human prostate carcinoma), HT29 (human colon carcinoma), and SKMES (human lung carcinoma) and A2780AD10 (human ovarian carcinoma).

Three A₃ receptor antagonists have been tested for their Degree of Growth Inhibitory activity and enhancement of inhibitory growth function of known anti-neoplastic agents. Table 6 indicates receptor binding assay results for three A₃ receptor antagonist compounds. Structures of the three compounds are as follow:

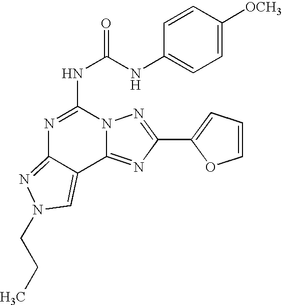

MRE3008F20: 5-[[(4-Methoxyphenyl)amino]carbonyl]amino-8-propyl-2-(2-furyl)-pyrazolo[4,3-e]1,2,4-triazolo[1,5-c]pyrimidine

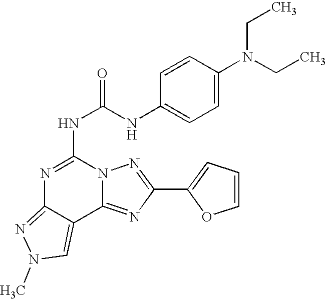

IL-10: N-1-(4-diethylamino-phenyl)-N′—S-[8-methyl-2-(2-furyl)-pyrazolo[4,3-e]1,2,4-triazolo[1,5-c]pyrimidine]-urea

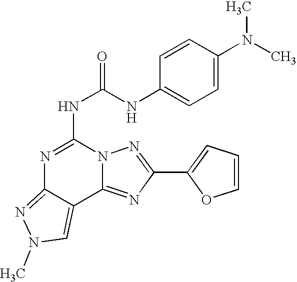

IL-11: N-1-(4-dimethylamino-phenyl)-N′-5-[8-methyl-2-(2-furyl)-pyrazolo[4,3-e]1,2,4-triazolo[1,5-c]pyrimidine]-urea

TABLE 6
Binding Affinity at rA₁, rA₂ and hA₃ Adenosine Receptors

| | rA₁ (Kᵢ, nM) | rA_{2A} (Kᵢ, nM) | hA₃ (Kᵢ, nM) | rA₁/hA₃ | rA_{2A}/hA₃ |
|---|---|---|---|---|---|
| MRE3008F20 | >10,000 | 1,993 | 0.29 | >34,482 | 6,872 |
| | | (1,658-2,397) | (0.27-0.32) | | |
| IL-10 | 580 | 523 | 27 | 21.5 | 19.4 |
| IL-11 | >10,000 | >10,000 | 15 | >6700 | >6700 |

Table 6 shows MRE3008F20, IL-10 and IL-11 to be potent, selective antagonists for the human adenosine A₃ receptor.

In addition to binding affinity studies of these three compounds, growth inhibitory studies were also performed. Results of growth studies of the compounds, uncombined with other compounds, are shown in Table 7. Table 7 also indicates the growth inhibitory activity of common anti-neoplastic agents.

TABLE 7
Growth Inhibitory Activity of A₃ Antagonists and Anti-Neoplastic Agents
Used Separately

| | IC₅₀ Values (μg/ml) ± Mean | | | |
|---|---|---|---|---|
| | Human | | Human | Human Lung |
| | Melanoma | Human Colon | Pancreatic Cancer | Carcinoma |
| Agent | A375 | Carcinoma HT29 | Panc-1 | SKMES |
| MRE3008F20 | 91.3 ± 1.5 | 39.4 ± 7.7 | 15.8 ± 13.5 | 19 ± 1.2 |
| IL-10 | 11.9 ± 0.4 | 10 ± 0.7 | 3.9 ± 3.3 | 5.9 ± 0.3 |
| IL-11 | 49.3 ± 40.7 | 56.5 ± 8.3 | 8 ± 5.7 | 36.3 ± 31.5 |
| irinotecan HCL | 0.88 ± 0.19 | 1.05 ± 0.07 | 1.12 ± 0.29 | 1.23 ± 0.36 |
| paclitaxel | 0.004 ± 0.0002 | 0.00112 ± 0.0003 | 0.0027 ± 0.0003 | 0.0026 ± 0.0007 |
| docetaxel | 0.0002 ± 0.00007 | 0.00024 ± 0.00005 | 0.00037 ± 0.00031 | 0.00084 ± 0.00037 |
| vinblastine | 0.0016 | 0.0019 ± 0.0004 | 0.0018 ± 0.0008 | 0.0019 ± 0.0009 |

Compounds MRE3008F20, IL-10 and IL-11 were obtained from King Pharmaceutical, Inc. Irinotecan HCl (Camptosar®; 20 mg/ml) was obtained from Pharmacia & Upjohn Co. Paclitaxel (Taxol®, 6 mg/ml) was obtained from Bristol-Myers Squibb, Co. Docetaxel (Taxotere®) was obtained from Rhone-Poulenc Rorer. Vinblastine sulfate salt was obtained from Sigma Chemical Co., (V1377). Irinotecan was diluted with culture medium. All other agents were dissolved in 100% DMSO at appropriate concentrations. The DMSO stock solutions were diluted 100-fold with growth medium so that the final DMSO concentration was 1%. We have previously shown that DMSO has no effect on the growth of culture cells at concentrations up to 1%. MTT (3-[4,5-Dimethylthiazol-2-yl]2,5-diphenyltetrazolium bromide) was obtained from Sigma Chemical Co. RPMI-1640 medium, antibiotic antimycotic 100× consisting of 10,000 units/ml penicillin G sodium, 10,000 μg/ml streptomycin sulfate, 25 μg/ml Amphotericin B (fungizone), glutamine (200 mM), HEPES buffer (1 M), gentamicin (50 mg/ml), sodium bicarbonate (7.5%), and fetal bovine serum were obtained from GibcoBRL. The complement in fetal bovine serum was inactivated at 56° C. for 30 min.

The human melanoma A375, human lung carcinoma SKMES, colon carcinoma HT29, and pancreatic cancer Panc-1 cell lines were originally obtained from the ATCC (American Type Culture Collection) and have been maintained in RPMI-1640 medium supplemented with 100 units/ml penicillin G sodium, 100 μg/ml streptomycin sulfate, 0.25 μg/ml amphotericin B (fungizone), 2 mM glutamine, 10 mM HEPES, 25 μg/ml gentamycin, and 10% heat-inactivated fetal bovine serum.

Most of the agents showed a broad spectrum and equally potent growth inhibitory activity against these four histologically distinct human tumor cell lines (A375 melanoma, HT29 colon carcinoma, Panc-1 pancreatic carcinoma, and SKMES lung carcinoma). The IC₅₀ values were approximately the same against all four cell lines. Interestingly, HT29 is approximately 30-fold more refractory than the A375 cell line to doxorubicin (IC₅₀ value of 0.211 vs. 0.0064 μg/ml) and mitoxantrone (IC₅₀ value of 0.1 vs. 0.0032 μg/ml), respectively. It is possible that the HT29 cell line has an altered form of topoisomerase II, and is therefore more refractory to doxorubicin and mitoxantrone, which mediate their growth inhibitory activity by trapping topoisomerase II, DNA, and drug in ternary complexes.

7.1 Abundance of A₃ Receptors in Human Solid Tumors and Melanoma.

Inventors verified the concentration of adenosine A₃ receptors in human cancer cells that is elevated in the cancerous tumors compared to non-cancerous tumors or normal tissue. Table 8 and FIG. 1 indicate elevated levels of adenosine A₃ receptors found by the inventors in human A375 (human melanoma), Panc-1 (human pancreatic carcinoma), MX-1 (human breast carcinoma), PC-1 (human prostate carcinoma), HT29 (human colon carcinoma), and SKMES (human lung carcinoma).

TABLE 8
Abundance of A₃ receptors in human solid tumors and
melanoma. Data shown are equilibrium binding parameters at 4° C.
expressed as dissociation constant, K_{D} (nM), and B_{MAX}
(fmol/mg protein) for [³H]MRE 3008F20 derived from saturation
experiments to human A₃ adenosine receptors
expressed in tumour tissues.

| Tumor types | K_{D} | B_{MAX} | FIG. |
|---|---|---|---|
| Human malignant | 1.9 ± 0.2 | 256 ± 31 | 1A |
| melanoma A375 | | | |
| Human colon DLD1 | 2.1 ± 0.3 | 434 ± 40 | 1B |
| Human pancreatic | 2.6 ± 0.3 | 249 ± 34 | 1C |
| MiaPaCa | | | |
| Human pancreatic | 2.7 ± 0.1 | 441 ± 46 | 1D |
| Panc1 | | | |
| Human breast tumor | 1.9 ± 0.2 | 435 ± 50 | 1E |
| MX1 | | | |
| Human lung squamour | 2.4 ± 0.6 | 169 ± 20 | 1F |
| carcinoma SKMES | | | |
| Human pancreatic PC3 | 3.1 ± 0.1 | 379 ± 40 | 1G |
| Human colon HT29 | 3.3 ± 0.3 | 213 ± 23 | 1H |

7.2 Human Melanoma A375

The growth inhibitory activity of paclitaxel against the human melanoma A375 cell line was determined in the absence or in the presence of 10 μg/ml of MRE3008F20 or 5 μg/ml each of IL-10 and IL-11 (Table 9). At these sub-cytotoxic concentrations, MRE3008F20, IL-10, and IL-11 (approximately 30-45% growth inhibition in the presence of A₃ antagonists alone), enhanced the growth inhibitory activity of paclitaxel by 8-12-fold; IC₅₀ values decreased from 0.0046 μg/ml (paclitaxel alone) to 0.0004-0.00054 μg/ml (paclitaxel plus MRE3008F20, IL-10, and IL-11).

TABLE 9
Growth Inhibitory Activity of A₃ Antagonists and Anti-Neoplastic
Agents Used Jointly With A375 Cells

| Anti-Neoplastic Agent | Antagonist | IC₅₀ | Enhancement |
|---|---|---|---|
| (Concentration Range) | (Concentration) | (μg/ml) | Factor |
| paclitaxel (0.0002-0.1 μg/ml) | None | 0.0046 | |
| paclitaxel (0.0002-0.1 μg/ml) | MRE3008F20 | 0.00054 | 8.5 |
| | (10 μg/ml) | | |
| paclitaxel (0.0002-0.1 μg/ml) | IL-10 (5 μg/ml) | 0.0005 | 9.2 |
| paclitaxel (0.0002-0.1 μg/ml) | IL-11 (5 μg/ml) | 0.0004 | 11.5 |

It was also determined that IL-10 and IL-11 enhance the growth inhibitory activity of paclitaxel in a concentration-dependent manner. Results of further testing of the enhancement effects with taxane family compounds is given in Table 10. Both of the common taxane compounds paclitaxel (taxol™) and docetaxel (taxotere™) are included.

Table 10 illustrates concentration-dependencies for concentrations of 1, 3, and 10 μg/ml for MRE3008F20 and for concentrations of 0.5, 1.5, and 5 μg/ml for compounds IL-10 and IL-11. MRE3008F20 enhanced the growth inhibitory activity of paclitaxel in a concentration-independent manner (enhancement factor=6.4-7.1 at all three concentrations). On the other hand, IL-10 and IL-11 enhanced the growth inhibitory activity of paclitaxel in a concentration-dependent manner. This testing also indicated that compounds MRE3008F20, IL-10 and IL-11 have enhancement factors with docetaxel that are indicative of a synergistic effect.

TABLE 10
Growth Inhibitory Activity of Antagonists and Taxane Compounds used
Jointly with A375 Cells

| Anti-Neoplastic Agent | A₃ Antagonist | | Enhancement |
|---|---|---|---|
| (Concentration Range) | (Concentration) | IC₅₀ (μg/ml) | Factor |
| paclitaxel (0.0001-0.05 μg/ml) | None | 0.0042 | |
| paclitaxel (0.0001-0.05 μg/ml) | MRE3008F20 (1 μg/ml) | 0.00059 | 7.1 |
| paclitaxel (0.0001-0.05 μg/ml) | MRE3008F20 (3 μg/ml) | 0.00066 | 6.4 |
| paclitaxel (0.0001-0.05 μg/ml) | MRE3008F20 (10 μg/ml) | 0.00059 | 7.1 |
| paclitaxel (0.0001-0.05 μg/ml) | none | 0.0044 | |
| paclitaxel (0.0001-0.05 μg/ml) | IL-10 (0.5 μg/ml) | 0.0012 | 3.7 |
| paclitaxel (0.0001-0.05 μg/ml) | IL-10 (1.5 μg/ml) | 0.00061 | 7.2 |
| paclitaxel (0.0001-0.05 μg/ml) | IL-10 (5 μg/ml) | 0.0005 | 8.8 |
| paclitaxel (0.0001-0.05 μg/ml) | none | 0.0044 | |
| paclitaxel (0.0001-0.05 μg/ml) | IL-11 (0.5 μg/ml) | 0.0014 | 3.1 |
| paclitaxel (0.0001-0.05 μg/ml) | IL-11 (1.5 μg/ml) | 0.00067 | 6.6 |
| paclitaxel (0.0001-0.05 μg/ml) | IL-11 (5 μg/ml) | 0.00046 | 9.6 |
| docetaxel (0.00001-0.005 μg/ml) | none | 0.000038 | |
| docetaxel (0.00001-0.005 μg/ml) | MRE3008F20 (1 μg/ml) | 0.0000006 | 63.3 |
| docetaxel (0.00001-0.005 μg/ml) | MRE3008F20 (3 μg/ml) | 0.0000055 | 6.9 |
| docetaxel (0.00001-0.005 μg/ml) | MRE3008F20 (10 μg/ml) | 0.0000072 | 5.3 |
| docetaxel (0.00001-0.005 μg/ml) | none | 0.000021 | |
| docetaxel (0.00001-0.005 μg/ml) | IL-10 (0.5 μg/ml) | 0.0000025 | 8.4 |
| docetaxel (0.00001-0.005 μg/ml) | IL-10 (1.5 μg/ml) | 0.0000023 | 9.1 |
| docetaxel (0.00001-0.005 μg/ml) | IL-10 (5 μg/ml) | 0.0000072 | 2.9 |
| docetaxel (0.00001-0.005 μg/ml) | none | 0.000046 | |
| docetaxel (0.00001-0.005 μg/ml) | IL-11 (0.5 μg/ml) | 0.0000087 | 5.3 |
| docetaxel (0.00001-0.005 μg/ml) | IL-11 (1.5 μg/ml) | 0.000005 | 9.2 |
| docetaxel (0.00001-0.005 μg/ml) | IL-11 (5 μg/ml) | 0.0000071 | 6.5 |

7.3 Human Lung Carcinoma SKMES

In human lung carcinoma SKMES, all three A₃ antagonists enhance the growth inhibitory activity of paclitaxel, docetaxel, irinotecan and vindesine (Table 11).

TABLE 11
Growth Inhibitory Activity of A₃ Antagonists and Anti-Neoplastic Agents
used Jointly with SKMES Cells

| Anti-Neoplastic Agent | A₃ Antagonist | | Enhancement |
|---|---|---|---|
| (Concentration Range) | (Concentration) | IC₅₀ (μg/ml) | Factor |
| paclitaxel (0.0001-0.05 μg/ml) | None | 0.0033 | |
| paclitaxel (0.0001-0.05 μg/ml) | MRE3008F20 (1 μg/ml) | 0.0008 | 4.1 |
| paclitaxel (0.0001-0.05 μg/ml) | IL-10 (0.5 μg/ml) | 0.0012 | 2.8 |
| paclitaxel (0.0001-0.05 μg/ml) | IL-11 (0.5 μg/ml) | 0.00078 | 4.2 |
| docetaxel (0.00001-0.005 μg/ml) | none | 0.00047 | |
| docetaxel (0.00001-0.005 μg/ml) | MRE3008F20 (1 μg/ml) | 0.00013 | 3.6 |
| docetaxel (0.00001-0.005 μg/ml) | IL-10 (0.5 μg/ml) | 0.00017 | 2.8 |
| docetaxel (0.00001-0.005 μg/ml) | IL-11 (0.5 μg/ml) | 0.00025 | 1.9 |
| docetaxel (0.00001-0.005 μg/ml) | none | 1.17 | |
| irinotecan HCL (0.04-20 μg/ml) | MRE3008F20 (1 μg/ml) | 0.57 | 2.1 |
| irinotecan HCL (0.04-20 μg/ml) | IL-10 (0.5 μg/ml) | 0.67 | 1.7 |
| irinotecan HCL (0.04-20 μg/ml) | IL-11 (0.5 μg/ml) | .061 | 1.9 |
| vinblastine (0.00001-0.005 μg/ml) | none | 0.0015 | |
| vinblastine (0.00001-0.005 μg/ml) | MRE3008F20 (1 μg/ml) | 0.0013 | 1.2 |
| vinblastine (0.00001-0.005 μg/ml) | IL-10 (0.5 μg/ml) | 0.0011 | 1.4 |
| vinblastine (0.00001-0.005 μg/ml) | IL-11 (0.5 μg/ml) | 0.00094 | 1.6 |

In comparing Table 11 with the results obtained with A375 human melanoma, the magnitude of enhancement for the taxane compounds in SKMES cells is less than that observed in A375 melanoma. These differences in absolute magnitude of synergism may be related to the activity of P-gp in the different tumors under the test conditions employed. However, the enhancement between A₃ antagonists and taxane compounds is consistently observed. Enhancement in SKMES cells is also consistent with a mechanism of inhibiting P-gp, in the case of vinblastine, and inhibiting MRP, as in the case of irinotecan HCL.

7.4 Human Colon Carcinoma HT29

The growth inhibitory activity of paclitaxel and docetaxel against the human colon carcinoma HT29 cell line was determined in the absence or in the presence of 1 μg/ml of MRE3008F20 or 0.5 μg/ml each of IL-10 and IL-11 (Table 12). It was found that A₃ antagonists enhance the growth inhibitory activity of paclitaxel, docetaxel, doxorubicin and irinotecan.

TABLE 12
Growth Inhibitory Activity of A₃ Antagonists and Taxane Compounds Used
Jointly With HT29 Cells

| Anti-Neoplastic (Concentration | A₃ Antagonist | | Enhancement |
|---|---|---|---|
| Range) Agent | (Concentration) | IC₅₀ (μg/ml) | Factor |
| paclitaxel (0.001-0.05 μg/ml) | none | 0.0025 | |
| paclitaxel (0.001-0.05 μg/ml) | MRE3008F20 (1 μg/ml) | 0.00085 | 2.9 |
| paclitaxel (0.001-0.05 μg/ml) | IL-10 (0.5 μg/ml) | 0.00100 | 2.5 |
| paclitaxel (0.001-0.05 μg/ml) | IL-11 (0.5 μg/ml) | 0.00099 | 2.5 |
| docetaxel (0.0001-0.005 μg/ml) | none | 0.000018 | |
| docetaxel (0.0001-0.005 μg/ml) | MRE3008F20 (1 μg/ml) | 0.0000012 | 15.0 |
| docetaxel (0.0001-0.005 μg/ml) | IL-10 (0.5 μg/ml) | 0.0000033 | 5.5 |
| docetaxel (0.0001-0.05 μg/ml) | IL-11 (0.05 μg/ml) | 0.0000028 | 6.4 |
| doxorubicin (0.0002-0.1 μg/ml) | none | 0.46 | |
| doxorubicin (0.0002-0.1 μg/ml) | MRE3008F20 (1 μg/ml) | 0.28 | 1.6 |
| doxorubicin (0.0002-0.1 μg/ml) | IL-10 (0.5 μg/ml) | 0.31 | 1.5 |
| doxorubicin (0.0021-0.1 μg/ml) | IL-11 (0.5 μg/ml) | 0.38 | 1.2 |
| irinotecan HC1 (0.04-20 μg/ml) | none | 0.96 | |
| irinotecan HC1 (0.04-20 μg/ml) | MRE3008F20 (1 μg/ml) | 0.53 | 1.8 |
| irinotecan HC1 (0.04-20 μg/ml) | IL-10 (0.5 μg/ml)) | 0.77 | 1.2 |
| irinotecan HC1 (0.04-20 μg/ml) | IL-11 (0.5 μg/ml) | 0.85 | 1.1 |

7.5 Human Pancreatic Cancer PANC-1

In human pancreatic cancer Panc-1, A₃ antagonists potentiated the growth inhibitory activity of taxane family compounds paclitaxel and docetaxel (Table 13). However, the potentiation observed is of a smaller magnitude compared to that observed in A375 melanoma.

TABLE 13
Growth Inhibitory Activity of A₃ Antagonists and Taxane Compounds Used
Jointly With Panc-1 Cells

| Anti-Neoplastic (Concentration | A₃ Antagonist | | Enhancement |
|---|---|---|---|
| Range) Agent | (Concentration) | IC₅₀ (μg/ml) | Factor |
| paclitaxel (0.0001-0.005 μg/ml) | None | 0.0029 | |
| paclitaxel (0.0001-0.05 μg/ml) | MRE3008F20 (1 μg/ml) | 0.0015 | 1.9 |
| paclitaxel (0.0001-0.005 μg/ml) | IL-10 (0.5 μg/ml) | 0.0017 | 1.7 |
| paclitaxel (0.0001-0.05 μg/ml) | IL-11 (0.5 μg/ml) | 0.0016 | 1.8 |
| docetaxel (0.00001-0.005 μg/ml) | none | 0.00068 | |
| docetaxel (0.00001-0.005 μg/ml) | MRE3008F20 (1 μg/ml) | 0.00038 | 1.8 |
| docetaxel (0.00001-0.005 μg/ml) | IL-10 (0.5 μg/ml) | 0.00047 | 1.4 |
| docetaxel (0.00001-0.005 μg/ml) | IL-11 (0.5 μg/ml) | 0.00050 | 1.4 |

It is seen from the above data and tables that the use of high affinity adenosine A₃ receptor antagonists in conjunction with chemotherapeutic cancer agents result in a notable enhancement effect for many of the agents.

7.6 MDR Cancer Cell Treatments

Having noted that chemotherapeutic cancer agents can be selected for A₃ antagonist enhancement from the form of MDR they are associated with, the inventors have further established that high affinity A₃ adenosine receptor antagonists can be used to counter P-gp and MRP associated multi-drug resistance.

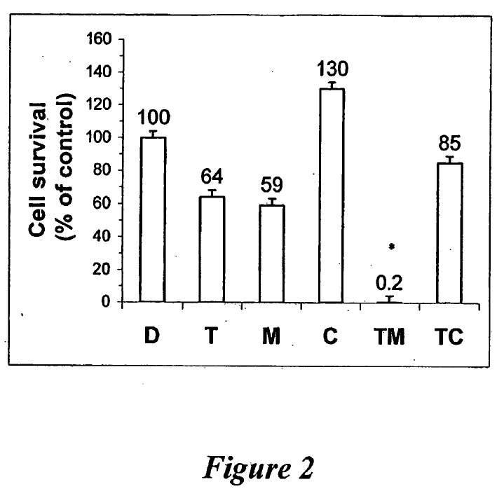

At first, inventors evaluated whether A₃ adenosine receptor could protect the cell by the toxic effect of conventional chemotherapeutic drug. Colony formation assay experiments were performed on A375 melanoma cells treated with increasing concentration of paclitaxel (0.25-75 μg/ml). A₃ stimulation is achieved with the selective agonist CI-IB-MECA while A₃ blockade is obtained with the selective antagonist MRE 3008F20. Colony formation of A375 cells is abolished when both paclitaxel (0.75 μg/ml) and MRE 3008F20 (10 μM) are applied whereas colony formation is only partly decreased when paclitaxel alone or MRE 3008F20 alone are applied. (FIG. 2)

Colony formation of A375 cells is increased of about 30% when the adenosine A₃ agonist CI-IB-MECA (10 μM) is applied. This is seen in the “C” bar of FIG. 2 being 130% of the DMSO control bar “D” FIG. 2 further shows that increased colony formation occurs when CI-IB-MECA is combined with the taxane family compound paclitaxel (0.75 μg/ml). When paclitaxel alone is added (“T” bar of FIG. 2), colony formation is 64% of the control. Colony formation is increased 32% to 85% of control when the CI-IB-MECA is combined (“TC” bar of FIG. 2).

Using the adenosine A₃ antagonist MRE 3008F20 (10 μM) alone decreases colony formation to 59% of control (“M” bar of FIG. 2). Surprisingly, when the A₃ antagonist is combined with the taxane compound, virtually all colony formation ceases (“TM” bar of FIG. 2). This clearly identifies the synergistic nature of combining A₃ antagonists with chemotherapeutic agents. In comparison to the surprising result of 0.2% colony formation, a geometrical combination predicts a result of 38%.

One explanation for these results is that A₃ receptors trigger a pro-survival signal, not able to restore colony formation ability of A375 cells treated with the taxane paclitaxel, while the blockage of As increased paclitaxel mediated deleterious effects (p<0.01, “TM” bar versus “T” bar of FIG. 2).

Subsequent to colony formation experiments, the inventors also analysed the ability of A₃ adenosine receptor antagonists to enhance chemotherapeutic effects by performing an acute treatment of A375 cells with the taxane family compound paclitaxel and the vinca alkaloid vindesine. Cell proliferation and apoptosis are quantified by flow cytometer analysis after propidium-iodide (PI) DNA staining. At 24 hours post-exposure, paclitaxel and vindesine induced a dose dependent cell accumulation into G₂/M cell cycle phases and a parallel decrease of the G₁ population.

To quantify the effect of paclitaxel and vindesine to alter cell proliferation the inventors determined the concentration exerting the 50% of the G₂/M accumulation (EC₅₀). When exposed to increasing concentrations of chemotherapeutic drugs, EC₅₀ are 16.60±2.00 ng/ml and 1.90±0.20 nM for paclitaxel and vindesine, respectively (means of four experiments). Analyses further show that EC₅₀ of paclitaxel and vindesine for decrease of G₁ population are not significantly changed respect to EC₅₀ of G₂/M arrest. The S-phase population, representative of replicating DNA, is not appreciably changed.

Additional experiments quantified the sub-G₁ population, representative of cells undergoing apoptosis. In A375 cells, the percentage of apoptotic cells increases progressively with paclitaxel concentration, reaching the maximum value (ranging from 35 to 53% on different experiments) at 5 ng/ml. An increase of the paclitaxel concentration results in a decrease of A375 cells at sub-G₁. The concentration exerting the maximal apoptosis (EC_{MAX}) is 6.00±0.63 ng/ml (mean of four experiments). Similarly, experiments performed with vindesine obtain an EC_{MAX} value of 3.54±0.42 nM (mean of four experiments).

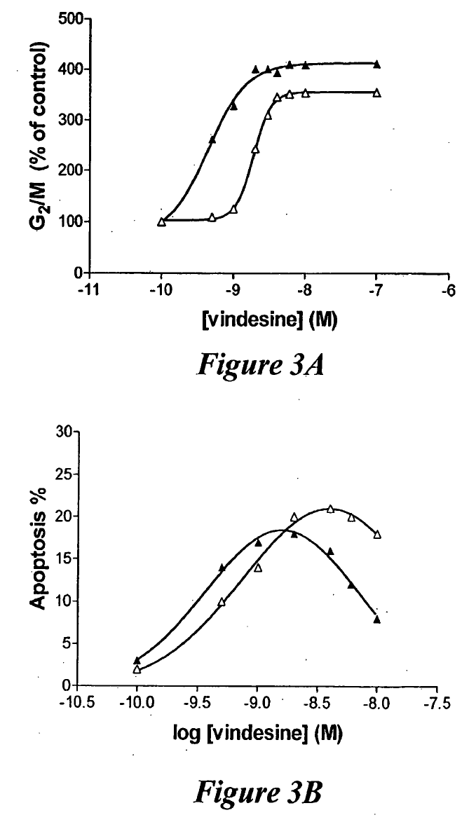

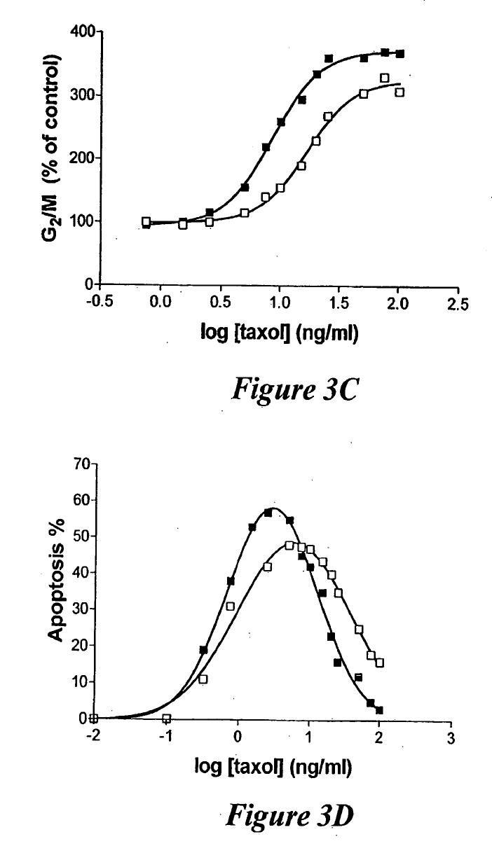

A375 cells treated with paclitaxel or with vindesine with or without the A₃ adenosine receptor selective A₃ antagonist MRE 3008F20 demonstrate enhancement. FIGS. 3A and 3C show that MRE 3008F20 (10 μM) improved vindesine and paclitaxel ability to alter cell proliferation: MRE 3008F20 reduced paclitaxel and vindesine EC₅₀ of 1.9 and 4.0 fold, respectively. Similar results were obtained analysing EC₅₀ values calculated for the G₁ population.

Furthermore, MRE 3008F20 (10 μM) reduced EC_{MAX} of 2.0 and 2.1 fold, for paclitaxel and vindesine, respectively (FIG. 3B and 3D).

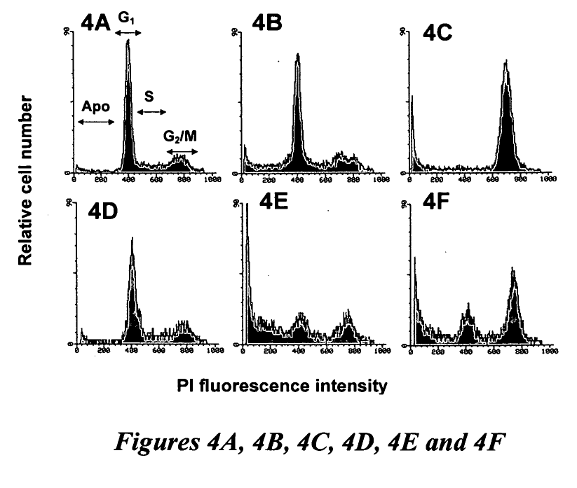

Representative flow cytometry profiles of DNA content in A375 cells (Apo (sub diploid cells), G₁, S and G₂/M phases) are shown in FIG. 4. FIG. 4C shows that, under both treatments (A₃ antagonist MRE3008F20 plus vinca alkaloid vindesine), vindesine response increased as cells progressed from G₁ to G₂/M phases respect to vindesine-treated cells alone (FIG. 4B). Similar results were obtained with the taxane compound paclitaxel (FIG. 4E-F).

To verify whether this enhancement activity was not related to toxic contaminants present in the MRE3008F20 solution but due to A₃ adenosine receptor specific blockade, inventors quantified the ability of other adenosine receptor antagonists to enhance A375 cell responses to vindesine and paclitaxel.

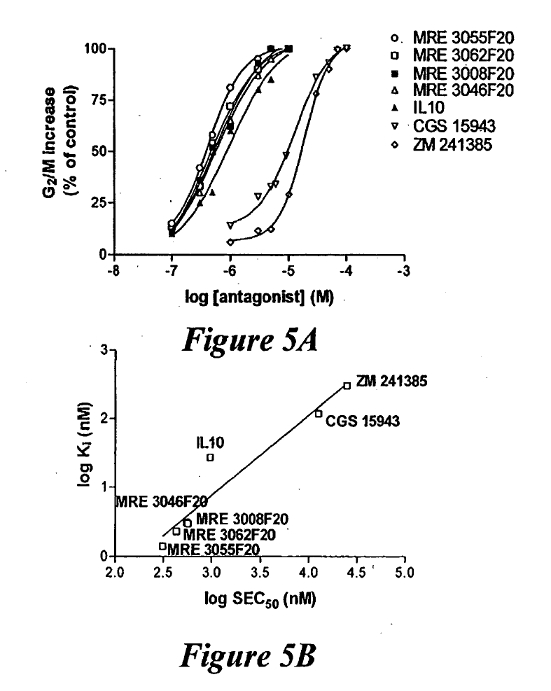

A375 cells were treated with vindesine (1 nM) with increasing concentrations of adenosine receptor antagonists (MRE3055F20, MRE3062F20, MRE3046F20, MRE3008F20, IL-10, CGS 15943, ZM 241385). As illustrated in FIG. 5A, the order of potency of adenosine antagonists to enhance Vindesine effect was: MRE3055F20 (highest)>MRE3062F20>MRE 3046F20>MRE3008F20>IL-10>CGS15943>ZM241385 (lowest). The concentrations exerting the 50% of the enhancing activity (SEC₅₀) are reported in Table 14 as the mean of four experiments. SEC₅₀ values are in good agreement with inhibitory equilibrium binding constants (Ki) observed in binding experiments for the adenosine A₃ receptor (FIG. 5B).

TABLE 14
Adenosine receptor antagonist parameters of enhancing activity
to vindesine and binding affinity to A₃ adenosine receptor in A375 cells.

| | SEC₅₀ | Ki |
|---|---|---|
| | (μM) | (nM) |
| MRE 3055F20 | 0.31 ± 0.03 | 1.4 ± 0.2 |
| MRE 3062F20 | 0.43 ± 0.04 | 2.3 ± 0.2 |
| MRE 3046F20 | 0.57 ± 0.06 | 3.0 + 0.3 |
| MRE 3008F20 | 0.55 ± 0.06 | 3.1 + 0.3 |
| IL-10 | 0.97 ± 0.10 | 30.0 ± 2.5 |
| CGS 15943 | 12.60 ± 1.41 | 118 ± 12 |
| ZM 241385 | 25.00 ± 2.23 | 270 ± 25 |

SEC₅₀: adenosine receptor antagonist dose that induces 50% of the enhancing activity to vindesine (1 nM), calculated on G₂/M accumulation dose response curve. Ki equilibrium constant of binding affinity at human A₃ adenosine receptor. Data represents the mean of four independent experiments.

To verify the role of A₃ receptor antagonists on paclitaxel and vindesine mediated alteration of cell proliferation and apoptosis, testing was performed with additional cell lines. Cell lines were selected with a pattern of surface adenosine receptor expression similar to A375 cell line, namely HeL023, Jurkat, NCTC2544 and PBMC blasts. Experimental conditions are similar to those for the A375 cell testing. The effect upon EC₅₀ values of vindesine and paclitaxel mediated cell cycle distribution alteration (G₂/M accumulation) under treatment with the A₃ antagonist MRE 3008F20 is given in Table 15. The variation in the level of apoptosis as reflected in EC₅₀ values are also indicated.

In Table 15, the HeLa23 cell line has the lowest sensitivity to paclitaxel and vindesine alone. Interestingly, HeLa23 cells also show the greatest amount of enhancement by MRE3008F20 co-treatment.

TABLE 15
Induction of G₂/M accumulation and apoptis of different cells by paclitaxel
and vindesine with or without A₃ receptor antagonist MRE3008F20 treatment

| | A375 | | HeLa23 | | Jurkat | |
|---|---|---|---|---|---|---|
| | paclitaxel | vindesine | paclitaxel | vindesine | paclitaxel | vindesine |
| | EC₅₀ | EC₅₀ | EC₅₀ | EC₅₀ | EC₅₀ | EC₅₀ |
| DMSO | 16.60 ± 2.00 | 1.90 ± 0.20 | 50.60 ± 5.30 | 25.80 ± 3.25 | 2.96 ± 0.31 | 0.93 ± 0.08 |
| MRE 3008F20 | 8.60 ± 0.81* | 0.48 ± 0.05* | 4.10 ± 0.40* | 0.71 ± 0.08* | 2.52 ± 0.30 | 0.37 ± 0.04* |

| | EC_{MAx} | EC_{MAx} | EC_{MAx} | EC_{MAx} | EC_{MAx} | EC_{MAx} |
|---|---|---|---|---|---|---|
| DMSO | 6.00 ± 0.63 | 3.54 ± 0.42₂ | 33.42 ± 3.55 | 33.11 ± 4.02 | 3.32 ± 0.52 | nd |
| MRE 3008F20 | 3.04 ± 0.32* | 1.66 ± 0.20 | 3.19 ± 0.30* | 1.05 ± 0.09* | 2.29 ± 0.31* | nd |

| | |
|---|---|
| NCTC 2544 | PBMC |

| | paclitaxel | vindesine | paclitaxel | vindesine |
|---|---|---|---|---|
| | EC₅₀ | EC₅₀ | EC₅₀ | EC₅₀ |
| DMSO | 2.69 ± 0.28 | 0.89 ± 0.08 | 5.30 ± 0.60 | 1.10 ± 0.90 |
| MRE 3008F20 | 2.41 ± 0.30 | 0.51 ± 0.06* | 5.30 ± 0.60 | 0.86 ± 0.09 |

| | EC_{MAx} | EC_{MAx} | EC_{MAx} | EC_{MAx} |
|---|---|---|---|---|
| DMSO | 2.69 ± 0.23 | 2.50 ± 0.27 | nd | nd |
| MRE 3008F20 | 2.55 ± 0.24 | 1.34 ± 0.15* | nd | nd |

Data represent the mean ± SE of four independent experiments. MRE3008F20 is 10 μM.

EC₅₀ values were obtained by analysing G₂/M accumulation dose response curve. EC_{MAX} values were obtained by analysing sub-G₁ accumulation dose response curve. Paclitaxel values have units of ng/ml. Vindesine values have units of nM. nd: not done. *P<0.01 vs DMSO; analysis was by ANOVA followed by Dunnett's test.

The greater relative enhancement in the HeL023 cell line may be related to a factor absent in other cell lines or related to the intracellular concentration of actives that is modulated by P-glycoprotein drug expulsion activity.

Rhodamine 123 (Rh123) retention, a P-gp functional assay, was studied in all cell lines. This assay was performed by incubating cells with Rh123 and determining Rh123 accumulation by measuring its fluorescence. The maximum load of Rh123 (F_{MAX}) was quantified at the end of treatment harvesting the cells and storing them at 4° C. to prevent any active Rh123 efflux. The P-gp drug efflux was allowed incubating Rh123-loaded cells in fresh new medium Rh123-free for 3 hours at 37° C. After this incubation, residual fluorescence (F_{RES}) was measured and compared to F_{MAX}.

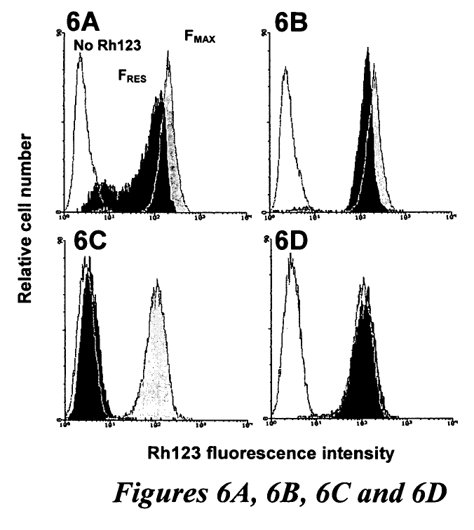

Results of the Rh123 retention in A375 cells is shown in FIG. 6A and FIG. 6B. FIG. 6A shows the flow chromatogram for Rh123 accumulation by A375 cells when the adenosine A₃ antagonist is not present. Two cell populations are found, characterised by an F_{RES} having a mean fluorescence intensity lower than F_{MAX}. The population with the lowest fluorescence, accounting for 26±5% of total cells, represents the cells expressing functional P-gp and having low intracellular level of Rh 123.

In contrast, FIG. 6B shows the A375 cellular accumulation of Rh123 in the presence of MRE3008F20 (10 μM). With the high affinity A₃ antagonist present, the response yielded a F_{RES} chromatogram comparable to F_{MAX}, consistent with a completely blockade of P-gp mediated Rh123 transport.

Results of the Rh123 retention in human herithro-leukemia HeL023 cells is shown in FIG. 6C and FIG. 6D. FIG. 6C shows that HeL023 cells have higher P-gp activity than A375 cells. F_{RES} (dark shaded area) is similar to the chromatogram obtained with cells not treated with Rh123 (autofluorescence). This is consistent with nearly all (100±1%) the cells expressing high level of functional P-gp.

With the addition of the A₃ antagonist MRE3008F20, HeL023 cells showed a P-gp inhibitor behaviour (FIG. 6D). FRES in the presence of MRE 3008F20, is similar to F_{MAX}. This is indicative of near total P-gp inhibition.

The Jurkat and NCTC2544 cells studied did not appear to have a significant change of Rh123 fluorescence after 3 hours of incubation at 37° C. (F_{RES} was similar to F_{MAX}) consistent with the absence or not detectable P-pg drug pumping activity in these cells.

To verify that the anti-P-gp activity of A₃ antagonists is not related to toxic contaminants present in the MRE3008F20 solution, the ability of other adenosine receptor antagonists to interfere with P-gp mediated drug-efflux was quantified. HeL023 cells were treated with adenosine receptor antagonists (IL-10, MRE3008F20, MRE3055F20, MRE3062F20, MRE3046F20, CGS15943 and ZM241385). Results are given in Table 16.

Table 16 shows the percentage of cells expressing P-gp activity (% of Rh123 negative cells) in the presence of adenosine receptor antagonists. The high affinity adenosine A₃ antagonists IL-10, MRE3008F20, MRE3055F20, MRE3062F20 and MRE3046F20 are strong inhibitors of P-gp activity. In contrast the low affinity antagonists, ZM241385 and CGS15943 had much lower effect on P-gp activity. This may be due to the higher A₃ affinity or due to the existence of a structure-activity relationship for the inhibition of P-gp drug expulsion activity mediated by adenosine receptor antagonists.

TABLE 16

| | Concentration | % of Rh123 |
|---|---|---|
| Treatment | (μM) | Negative Cells |
| DMSO | | 99.7 ± 1.2 |
| IL 10 | 10 | 0.4 ± 0.2 |
| 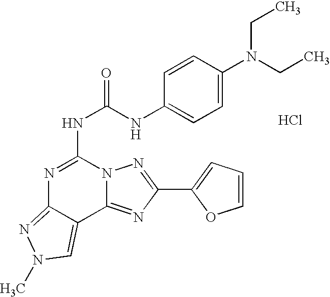 | | |
| MRE 3008F20 | 10 | 0.5 ± 0.3 |
| 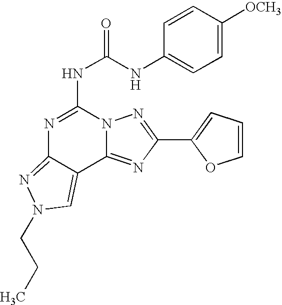 | | |
| MRE3055F20 | 10 | 0.8 ± 0.2 |
| 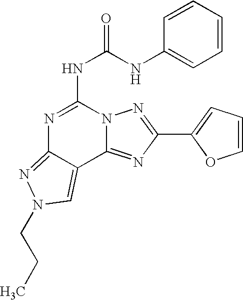 | | |
| MRE 3062F20 | 10 | 1.0 ± 0.2 |
| 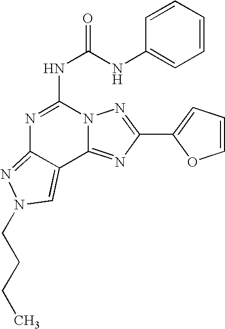 | | |
| MRE 3046F20 | 10 | 22.0 ± 1.0 |
| 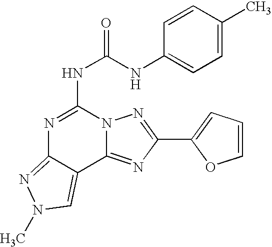 | | |
| CGS 15943 | 70 | 87.1 ± 1.0 |
| 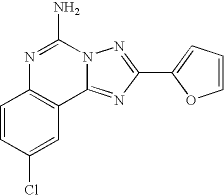 | | |
| ZM 241385 | 70 | 93.0 ± 1.1 |
| 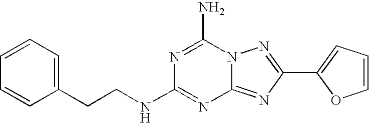 | | |

Table 16 data represents the mean of four independent experiments.

To underline the molecular mechanisms sustaining the A₃ antagonist mediated response to vindesine and paclitaxel anti-proliferative effects, signalling studies were performed.

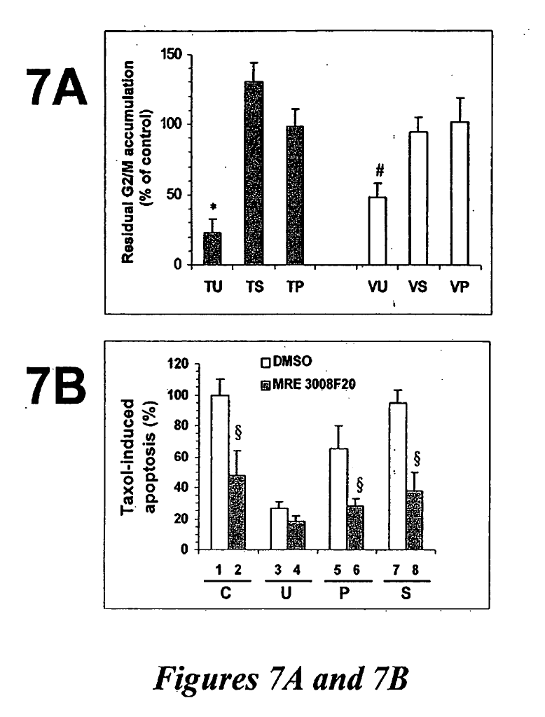

Previous research has related paclitaxel and Vinca alkaloid derivatives lethality to activation of mitogen-activated protein kinase (MAPK) family (Lieu in Sequence-dependent potentiation mol. Pharmacol. 2001). Three MAPK family members have been characterised thus far: ERKs (or p42/44^{MAPK}) JNK (or SAPK) and p38^{MAPK}. Signalling studies investigated the effect of ERKs and p₃₈^{MAPK} on MRE 3008F20 mediated enhancement to vindesine and to paclitaxel. The JNK pathway was not studied due to the lack of commercially available JNK inhibitor. Results are shown in FIG. 7A and FIG. 7B.

PD98059, a selective inhibitor of MEK1/2 (dudley dt, PNAS 92:7686, 1995), was used to inhibit the MEK pathways. U0126, an agent approximately 100-fold more potent than PD98059, was used as inhibitor of ERK activation. SB203580 selectively inhibits p38^{MAPK} activity (Young P R JBC 272:12116 1997). A375 cells were pretreated with PD98059 (20 μM), U0126 (30 μM), SB203580 (1 μM] or with DMSO (as control) and then challenged with vindesine (1 nM) or with paclitaxel (15 ng/ml). At 24 hours post treatment, cells were harvested and apoptosis and cell cycle were analysed.

The combination of PD98059, U0126 and SB203580 plus paclitaxel or vindesine did not alter the G₂/M accumulation rate respect to paclitaxel or vindesine alone. PD98059 and SB203580 failed to inhibit MRE3008F20 (10 μM) improved susceptibility of A375 cells to vindesine and paclitaxel. Results for PD98059 with paclitaxel is shown as bar “TP” of FIG. 7A and with vindesine is bar “VP”. The results for SB203580 plus paclitaxel is shown as bar “TS” of FIG. 7A and with vindesine is bar “VS”.

U0126 prevented MRE3008F20-induced accumulation in G₂/M by 23±5% and by 48±5% in presence of paclitaxel (bar “TU” of FIG. 7A) and vindesine (bar “VU” of FIG. 7A), respectively. This infers that the molecular mediator of enhancement activity was ERK.

The effects on selectively blocking the pathways is shown in FIG. 7B. Noted is a significant reduction of apoptosis induced by paclitaxel25 ng/ml in cells treated with PD98059 (35±6%, FIG. 7B, lane 5) and U0126 (73±10%, FIG. 7B, lane 3), whereas SB203580 did not exert any effect FIG. 7B, lane 7). However, PD98059 and SB203580 (FIG. 7B, lanes 6 and 8, respectively) failed to enhance the protective effect of MRE3008F20 in apoptosis induced by paclitaxel, as observed in presence of U0126 (FIG. 7B, lane 4).

These results provide confirmation that the MRE3008F20-induced reduction of apoptosis of paclitaxel is dependent on ERK activation. Additional testing demonstrated similar trends in HeL023, NCTC2544 and Jurkat cell lines (data not shown).

Further testing indicates that P-gp interference and ERK engagement can occur independently of each other. A375 cells were first treated with U0126 (30 μM) for 30 minutes and subsequently incubated with MRE3008F20 (10 μM). The P-gp activity is compared which those of cells treated with MRE3008F20 alone (for example, cells of FIG. 6B). U0126 failed to prevent MRE 3008F20 blockade of P-gp. This confirms that P-gp interference and ERK engagement can occur independently of each other.

Discussion: Results presented show that several adenosine receptor antagonists exert enhancing activity to chemotherapeutic agents. Noteworthy, this enhancing activity is A₃ adenosine receptor dependent. This pharmacological specificity was determined by an accurate Spermean's rank correlation between the dose exerting physiological effect (SEC₅₀ quantified on G₂/M accumulation) and the binding ability to A₃ adenosine receptors (Ki) of different adenosine receptor antagonists (r=0.96, FIG. 5B). A known and clear explanation is not yet available as to the mechanism of this enhancement. However, the high statistically significant Spermean's rank correlation coefficient between SEC₅₀ values in the G₂/M accumulation and receptor affinity values showed a highly significant positive correlation.

Results show that A₃ adenosine receptor antagonists enhance chemotherapeutic agent anti-proliferative and apoptotic effects. The A₃ adenosine receptor antagonists reduce EC₅₀ doses of chemotherapeutic drugs (quantified by analysing G₂/M accumulation rate) to 12.3, 1.9, 1.2-fold for paclitaxel and 36.3, 4.0, 2.5-fold for vindesine when challenged on HeL023, A375 and Jurkat cell lines, respectively. This enhancement activity was confirmed also when the apoptotic degree was evaluated: EC_{MAx} dose of chemotherapeutic drugs are reduced to 10.5, 2.0, 1.5-fold for paclitaxel on HeL023, A375 and Jurkat cells, respectively, and 31.5, 2.0-fold for vindesine on HeL023 and A375 cells, respectively.

The variable degree of EC₅₀ (and EC_{MAX}) value observed in different cell types suggest a cell type specific participation in drug-induced enhancement. Of note, the prerequisite of the drug activity is its delivery to the target site. However, efficiency of drug is limited by appearing resistance, i.e., lack of cell sensitivity to the administered drug. The tumor cells with multidrug resistance (MDR) phenotype are characterised by lowered intracellular accumulation of the compounds they are resistant to.

Moreover, most cell lines with MDR phenotype show the over-expression of 170 KDa membrane associated P-glycoprotein (P-gp) that acts as an energy-dependent efflux pump. It has been demonstrated that this protein plays an important role in the transport of toxic endogenous metabolites and it seems to be responsible for the decreased intracellular drug accumulation observed in resistant cells (Fojo A, Cancer Res 45:3002-7, 1985). Previous studies reported that melanoma and HeL heamopoietic cell line expressed functional P-gp

On the other hand, MRP and not P-gp transporter is expressed in Jurkat leukemia cells (T lymphocytic cell line). This is consistent with the results as HeL023 and A375 cells produced a P-gp efflux-activity whereas in Jurkat cells had low rhodamine 123 efflux. It is shown that adenosine A₃ antagonists are useful for enhancement in both P-gp expressing and MRP expressing cell lines.

The much lower ability of CGS15943 and ZM241385 to inhibit P-pg and MRP correlates with the lower adenosine A₃ affinity of these compounds. The high affinity A₃ antagonist compounds tested have greater potency in both enhancement and inhibiting P-gp and MRP drug resistance. This may be due to the higher affinity or to the molecular structure of such compounds. For example, the tested high affinity compounds have a phenyl-carbarnoyl-amino derivative in the N5 position of a 2-furylpyrazolo-triazolo-pyrimidine structure. CGS15943 and ZM241385 in addition to having much lower A₃ affinity, also have very different molecular structures.

7.7 Example Formulations

The following examples illustrate aspects of this invention but should not be construed as limitations. The symbols and conventions used in these examples are indented to be consistent with those used in the contemporary, international, chemical literature, for example, the Journal of the American Chemical Society and Tetrahedron.

### Example—Pharmaceutical Formulations

(A) Transdermal System—for 1000 patches

| Ingredients | Amount |
|---|---|
| Active compound | 100 g |
| Silicone fluid | 450 g |
| Colloidal silicon dioxide | 2 g |

The silicone fluid and active compound are mixed together and the colloidal silicone dioxide is added to increase viscosity. The material is then dosed into a subsequent heat sealed polymeric laminate comprised of the following: polyester release liner, skin contact adhesive composed of silicone or acrylic polymers, a control membrane which is a polyolefin, and an impermeable backing membrane made of a polyester multilaminate. The resulting laminated sheet is than cut into 10 sq. cm patches.

(B) Oral Tablet—For 1000 Tablets

| Ingredients | Amount |
|---|---|
| Active compound | 50 g |
| Starch | 50 g |
| Magnesium Stearate | 5 g |

The active compound and the starch are granulated with water and dried. Magnesium stearate is added to the dried granules and the mixture is thoroughly blended. The blended mixture is compressed into tablets.

(C) Injection—for 1000, 1 mL Ampules

| Ingredients | Amount | |
|---|---|---|
| Active compound | 10 | g |
| Buffering Agents | q.s. | |
| Propylene glycol | 400 | mg |
| Water for injection | q.s. 1000 | mL |

The active compound and buffering agents are dissolved in the propylene glycol at about 50°)C. The water for injection is then added with stirring and the resulting solution is filtered, filled into ampules, sealed and sterilized by autoclaving.

(D) Continuous Injection—for 1000 mL

| Ingredients | Amount | |
|---|---|---|
| Active compound | 10 | g |
| Buffering Agents | q.s. | |
| Water for injection | q.s. 1000 | mL |

The active compound and buffering agents are dissolved in water at about 50° C. The resulting solution is filtered, filled into appropriate administration container, sealed and sterilized.

(E) Topical Ointment—for 1000, 1 g packs

| Ingredients | Amount | |
|---|---|---|
| Active compound | 10 | g |
| White petroleum base | q.s. 990 | g |

The active compound is blended into the petrolatum base under sterile conditions and filled into 1 gram packs.

Although the present invention has been described in terms of specific embodiments, various substitutions of materials and conditions can be made as will be known to those skilled in the art. For example, other excipients may be utilized in preparing the pharmaceutical formulations. In addition, many of the active adenosine A₃ receptor antagonists contain one or more asymmetric centers and may therefore give rise to enantiomers and diastereomers as well as their racemic and resolved, enantiomerically pure or diastereomerically pure forms, and pharmaceutically acceptable salts thereof. It is often desirable that the adenosine A₃ receptor antagonists be given simultaneously with the cytotoxic agent. When this is the case, users of this invention may find it advantageous to combine the A₃ receptor antagonists with the cytotoxin into a single dosage form. These and other variations will be apparent to those skilled in the art and are meant to be included herein. The scope of the invention is only to be limited by the following claims:

7.8 Regulation of HIF-1A by Adenosine

Chemicals and Reagents: A375 melanoma, NCTC 2544 keratinocytes, U2OS osteosarcoma, U87MG glioblastoma human cells were obtained from American Tissue Culture Collection (ATCC). Tissue culture media and growth supplements were obtained from BioWhittaker. GasPak Pouch™ System was obtained from Becton Dickinson. Unless otherwise noted, all other chemicals were purchased from Sigma. Anti-HIF-1α and anti-HIF1β antibodies (mAb) were obtained from Transduction Laboratories (BD, Milano, Italy). U0126 (inhibitor of MEK-1 and MEK-2), SB202190 (inhibitor of p38 MAP kinase), Anti-ACTIVE®MAPK and anti-ERK 1/2 (pAb) were from Promega. Phospho-p38 and p38 MAP Kinase antibodies were from Cell Signaling Technology. Anti-Adenosine A₃ receptor (polyAb) was from Aviva Antibody Corporation.

Cell culture and hypoxia treatment: Cells were maintained in DMEM (A375), EMEM (NCTC 2544) or RPMI 1640 (U87MG, U2OS) medium containing 10% fetal calf serum, penicillin (100 U/ml), streptomycin (100 ug/ml), and L-glutamine (2 mM) at 37° C. in 5% CO₂/95% air. Cells were passaged two or three times weekly at a ratio between 1:5 and 1:10. Hypoxic exposure was in BBL™ GasPak pouch™ System (Becton Dickinson) that reduce oxygen concentration of less than 2% within 2 hours of incubation at 35° C.

[³H]-Thymidine incorporation: cell proliferation test: Cells were seeded in fresh medium with 1 uCi/ml of [³H]-Thymidine in DMEM containing 10% fetal calf serum, penicillin (100 U/ml), streptomycin (100 ug/ml), L-glutamine (2 mM). After 24 hours of labelling, cells were trypsinised, dispensed in 4 wells of a 96 well plate, and filtered through Whatman GF/C glass-fiber filters using a Micro-Mate 196 cell harvester (Packard Instrument Company). The filter bound radioactivity was counted on Top Count Microplate Scintillation Counter (efficiency 57%) with Micro-Scint 20.

Flow Cytometry analysis: A375 adherent cells were trypsinized, mixed with floating cells, washed with PBS and permeabilized in 70% (vol/vol) ethanol/PBS solution at 4° C. for at least 24 hours. The cells were washed with PBS and the DNA was stained with a PBS solution, containing 20 ug/ml of propidium iodide and 100 ug/ml of RNAse, at room temperature for 30 minutes. Cells were analysed with an EPICS XL flow cytometer (Beckman Coulter, Miami, Fla.) and the content of DNA was evaluated by the Cell-LISYS program (Becton-Dickinson). Cell distribution among cell cycle phases and the percentage of apoptotic cells were evaluated as previously described (Merighi 2002). Briefly, the cell cycle distribution is shown as the percentage of cells containing 2n (G0/G₁ phases), 4n (G₂ and M phases), 4n>x>2n DNA amount (S phase) judged by propidium iodide staining. The apoptotic population is the percentage of cells with DNA content lower than 2n.

Small interfering RNA (siRNA) design: To generate a small interfering RNA that targets A₃ receptor mRNA (siRNA_{A3}), eight oligonucleotides consisting of ribonucleosides, except for the presence of 2′-deoxyribonucleosides at the 3′ end, were synthesized and annealed, according to the recommendations of Elbashir (ref), to the manufacturer's instructions (Silencer™ siRNA Construction Kit, Ambion) and as previously described (Mirandola, 2004). For oligo-1, sense sequence: 5′-GCU UAC CGU CAG AUA CAA GUU-3′ (SEQ ID NO:1) and antisense 5′-CUU GUA UCU GAC GGU AAG CUU-3′ (SEQ ID NO:2). For oligo-2, sense sequence: 5′-GAC GGC UAA GUC CUU GUU UUU-3′ (SEQ ID NO:3) and antisense 5′-AAA CAA GGA CUU AGC CGU CUU-3′ (SEQ ID NO:4). For oligo-3, sense sequence: 5′-ACA CUU GAG GGC CUG UAU GUU-3′ (SEQ ID NO:5) and antisense 5′-CAU ACA GGC CCU CAA GUG UUU-3′ (SEQ ID NO:6). For oligo-4, sense sequence 5′-CCU GCU CUC GGA GGA UGC CUU-3′ (SEQ ID NO:7) and antisense 5′-GGC AUC CUC CGA GAG CAG GUU-3′ (SEQ ID NO:8). Target sequences were aligned to the human genome database in a BLAST search to ensure sequences without significant homology to other genes. The target sequences for oligo-1, oligo-2, oligo-3 and oligo-4 are localized at position 337, 679, 1009 and 1356 bases downstream of the start codon of A₃ receptor mRNA sequence (L20463), respectively.

Treatment of cells with siRNA: A375 cells were plated in six-well plates and grown to 50-70% confluence before transfection. Transfection of siRNA was performed at a concentration of 100 nM using RNAiFect™ Transfection Kit (Qiagen). Cells were cultured in complete media and at 24, 48 and 72 hours total RNA was isolated for Real-Time RT-PCR analysis of A₃ receptor mRNA and for Western blot analysis of A₃ receptor protein. At 48 hours from the transfection, A375 cells were serum starved for another 24 hours and then exposed to increasing concentrations of the A₃ adenosine receptor agonist Cl-IB-MECA for 4 hours in hypoxia. Total protein were then harvested for Western blot analysis. As control, cells were exposed to RNAiFect™ Transfection reagent without siRNA_{A3}. To quantify cell transfection efficiency we used siRNA-FITC labelled (Qiagen). After 24 hours of transfection, cells were tripsinized and resuspended in PBS for flow cytometry analysis. Fluorescence obtained from FITC-siRNA transfected cells was compared to autofluorescence generated by untransfected control.

Real-Time RT-PCR experiments: Total cytoplasmic RNA was extracted by the acid guanidinium thiocyanate phenol method (Chomczynski & Sacchi, 1987). Quantitative real-time RT-PCR assay (Higuchi, 1993) of HIF-1α and A₃ mRNA transcripts was carried out using gene-specific double fluorescently labelled TaqMan MGB probe (minor groove binder) in a ABI Prism 7700 Sequence Detection System (Applied Biosystems, Warrington Cheshire, UK). The following primer and probe sequences were used for real-time RT-PCR: A₃ forward primer, 5′-ATG CCT TTG GCC ATT GTT G-3′ (SEQ ID NO:9); A₃ reverse primer, 5′-ACA ATC CAC TTC TAC AGC TGC CT-3′ (SEQ ID NO:10); A₃ MGB probe, 5′-FAM-TCA GCC TGG GCA TC-TAMRA-3′ (SEQ ID NO:11); for the real-time RT-PCR of the HIF-1α gene the assays-on-demand™ Gene expression Product Accession No. NM 019058 was used (Applied Biosystems, Monza, Italy). The fluorescent reporter FAM and the quencher TAMRA are 6-carboxy fluorescein and 6-carboxy-N,N,N′,N′-tetramethylrhodamine, respectively. For the real-time RT-PCR of the reference gene the endogenous control human β-actin kit was used, and the probe was fluorescent-labeled with VIC™ (Applied Biosystems, Monza, Italy).

Western blotting: A375, NCTC 2544, U20S and U87MG cells were treated with adenosine or adenosine analogues and exposed to normoxia and hypoxia for different times (2-24 hours). Cells were harvested and washed with ice-cold PBS containing 1 mM sodium orthovanadate, AEBSF 104 mM, aprotinin 0.08 mM, leupeptin 2 mM, bestatin 4 mM, pepstatin A 1.5 mM, E-64 1.4 mM. Cells were then lysed in Triton lysis buffer. The protein concentration was determined using BCA protein assay kit (Pierce). Equivalent amounts of protein (35 ug) were subjected to electrophoresis on 7.5% sodium dodecyl sulfate-acrylamide gel. The gel was then electroblotted onto a nitrocellulose membrane. Membranes were blocked with 5% nonfat dry milk in PBS containing 0.1% Tween-20 and incubated with antibodies against HIF-1α (1:250 dilution) and HIF-1β (1:1000 dilution) in 5% nonfat dry milk in PBS/0.1% Tween-20 overnight at 4° C. Aliquots of total protein sample (50 ug) were analyzed using antibodies specific for phosphorylated (Thr183/Tyr185) or total p44/p42 MAPK (1:5000 dilution), phosphorylated (Thr180/Tyr182) or total p38 MAPK (1:1000 dilution) and for A₃ receptor (1 □g/ml dilution). Filters were washed and incubated for 1 hour at room temperature with peroxidase-conjugated secondary antibodies against mouse and rabbit IgG (1:2000 dilution). Specific reactions were revealed with the Enhanced Chemiluminescence Western blotting detection reagent (Amersham Corp., Arlington Heights, Ill.). The membranes were then stripped and reprobed with tubulin (1:250) to ensure equal protein loading.

Metabolic inhibitors: Cells were treated for 30 minutes with metabolic inhibitors or with drug vehicle (DMSO) prior to being challenged with adenosine or adenosine analogues. U0126 was used at 10 and 30 uM as inhibitor of MEK-1 and MEK-2 to prevent p44 and p42 MAPK activation. SB202190 was used at 1 and 10 uM as inhibitor of p38 MAPK.

Densitometry analysis: The intensity of each band in immunoblot assay was quantified using molecular analyst/PC densitometry software (Bio-Rad). Mean densitometry data from independent experiments were normalized to result in cells in the control. The data were presented as the mean±S.E., and analyzed by the Student's test.

Statistical analysis: All values in the figures and text are expressed as mean±standard error (S.E.) of n observation (with n≧3). Data sets were examined by analysis of variance (ANOVA) and Dunnett's test (when required). A P value less than 0.05 was considered statistically significant.

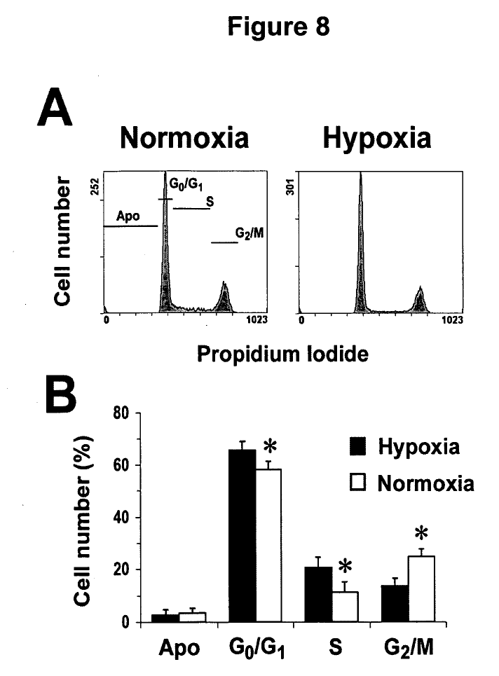

Results: Adenosine induces HIF-1α protein accumulation in hypoxia. We have evaluated the biological effect produced by a prolonged oxygen deprivation in the human A375 melanoma cell line. Viability and proliferation of A375 cells exposed to hypoxia for 24 hours were assessed analyzing the percentage of apoptotic cells and the distribution among the different phases of the cell cycle. We employed flow cytometry and DNA staining by propidium iodide for discrimination of cells in apoptosis, in G₀/G₁, S and G₂/M phases. The results indicate that hypoxia did not promote significant cell death while interfered with proliferation arresting melanoma cells in G₀/G₁ and S phases and reducing the number of cells in G₂/M (FIG. 8). These data were confirmed by using trypan blue exclusion, cell counts and [³H]-thymidine incorporation assay (data not shown).

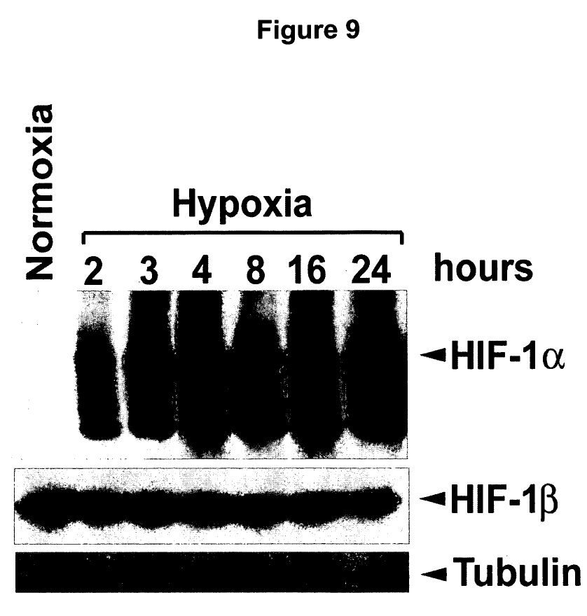

Exposure of A375 cells to hypoxia induced HIF-1α protein expression (FIG. 9). The hypoxic induction of HIF-1α was rapid, and an increase could be seen over the first 2 to 3 hours following hypoxic incubation. Maximal stimulation was obtained between 4-8 hours of incubation in oxygen-deprived conditions, while the levels of HIF-1α protein were somewhat lower with prolonged hypoxia. On the contrary, the levels of HIF-1β were not altered.

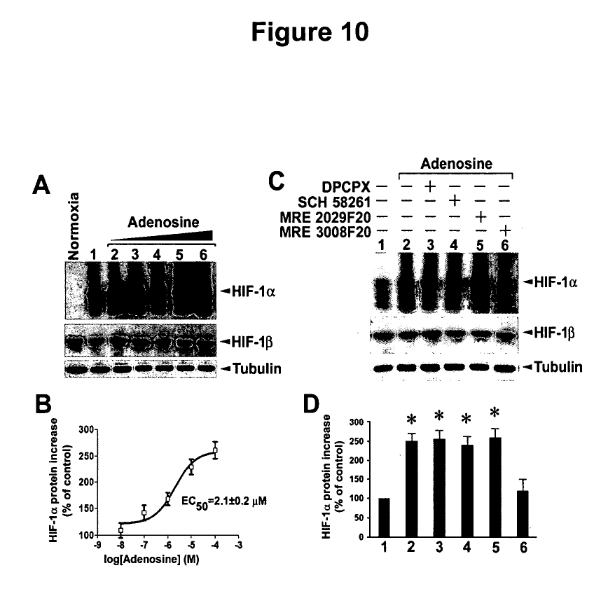

To study the effect of adenosine on the transcription factor HIF-1, A375 melanoma cells were treated for 4 hours with increasing concentrations of the nucleoside in hypoxic conditions. As observed in FIG. 10A, adenosine up-regulated HIF-1α protein expression in hypoxic melanoma cells. In particular, adenosine induced HIF-1α protein accumulation in a dose-dependent manner, with an EC₅₀=2.1±0.2 μM and a maximal increase of 2.6±0.2 fold at 100 μM (FIG. 10B). We did not observe any modulation of HIF-1β protein.

The family of adenosine receptors consists of four subtypes of G protein-coupled receptors, designed A₁, A_{2A}, A_{2B} and A₃. We have previously demonstrated that all four adenosine receptors are expressed in human melanoma A375 cells. To evaluate the functional role of adenosine receptor subtypes on HIF-1α protein expression under hypoxic conditions, we tested the effect of adenosine in combination with DPCPX (an A₁ receptor antagonist), SCH 58261 (a selective A_{2A} receptor antagonist), MRE 2029F20 (a selective A_{2B} receptor antagonist), and MRE 3008F20 (a selective A₃ receptor antagonist) (Baraldi 2004; Merighi 2001; Varani 2000). While the A₁, A_{2A} and A_{2B} receptor antagonists were not able to prevent adenosine-induced HIF-1α protein expression, the A₃ receptor antagonist MRE 3008F20 abrogated the adenosine-induced increase of HIF-1α protein expression (FIG. 10C-D). Furthermore, HIF-1β expression was unaffected by adenosine or by synthetic adenosine receptor antagonists. These results indicate that adenosine may increase HIF-1α protein expression via A₃ receptors.

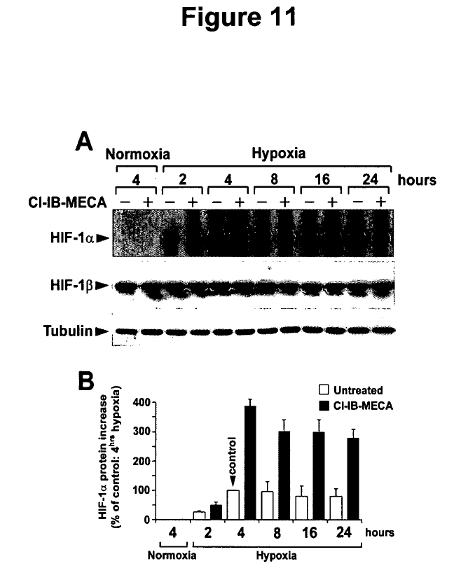

A₃ adenosine receptor induces HIF-1α protein accumulation in hypoxia. To investigate the involvement of A₃ receptors in the modulation of HIF-1α protein expression, we treated A375 cells with the selective A₃ receptor agonist Cl-IB-MECA. We performed a time-course experiment in which A375 cells were exposed to Cl-IB-MECA 100 nM for 2-24 hours. A₃ adenosine receptor stimulation did not promote HIF-1α protein accumulation in normoxia, while under hypoxic conditions HIF-1α protein expression was increased in a time-dependent manner (FIG. 11). In particular, HIF-1α increased from 2 hours and maximum peak levels were observed 4 hours after the addition of Cl-IB-MECA to the culture media. The prolonged A₃ receptor stimulation in hypoxia resulted in a minor effect in HIF-1α protein level modulation. As already observed with adenosine, also Cl-IB-MECA did not modify HIF-1β expression in normoxia and in hypoxia.

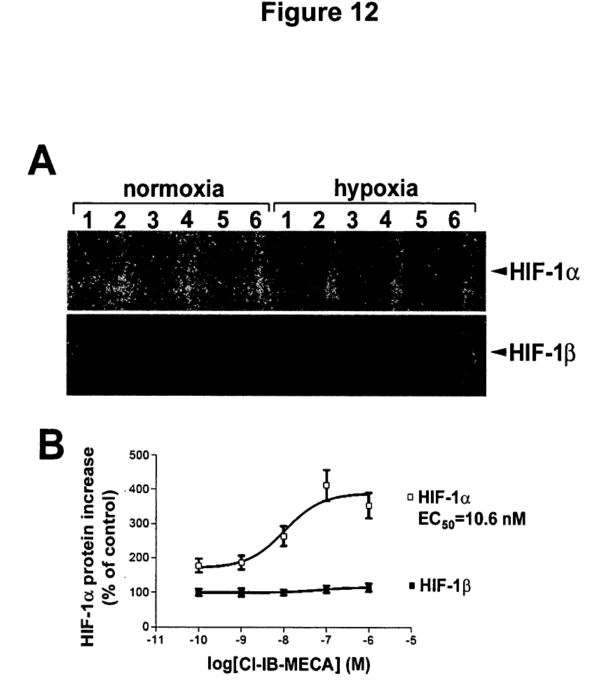

To characterize in more detail the induction of HIF-1α expression by A₃ receptor stimulation, A375 cells were treated with various concentrations of A₃ agonist for 4 hours. As expected, in normoxia the activation of A₃ receptors did not induce detectable levels of HIF-1α. On the contrary, in hypoxia Cl-IB-MECA induced HIF-1α protein accumulation in a dose-dependent manner (FIG. 12A), reproducing the effect produced by adenosine (FIG. 10). The maximum expression of HIF-1α protein was induced by Cl-IB-MECA at a concentration of 100 nM, with an EC₅₀=10.6±1.2 nM (FIG. 12B). In contrast, A₃ receptor stimulation did not affect the expression of HIF-1β protein, either in normoxia or in hypoxia.

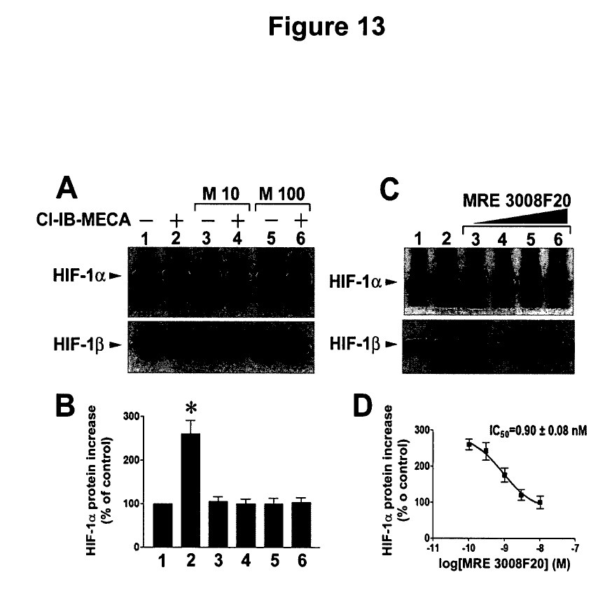

To better characterize the ability of A₃ receptor to significantly increase HIF-1α protein expression in hypoxia, we performed a series of experiments to evaluate the ability of A₃ selective receptor antagonists (MRE 3008F20 and MRE 3005F20) (Baraldi 2004) to prevent this effect. A375 cells were treated with increasing concentrations of MRE 3008F20 and MRE 3005F20 for 30 minutes with or without Cl-IB-MECA (10 and 100 nM) treatment. Both MRE 3008F20 and MRE 3005F20 (10 and 100 nM) were able to abrogate the effect of Cl-IB-MECA in HIF-1α modulation. As expected, neither MRE 3008F20 nor MRE 3005F20 had any effect on HIF-1β expression. FIG. 13A-B shows the results obtained with MRE 3008F20. Similar results were obtained with the antagonist MRE 3005F20 (data not shown).

We next investigated the effect of increasing concentrations of MRE 3008F20 on HIF-1α protein increase induced by a submaximal dose of Cl-IB-MECA. HIF-1α protein increase, induced by 10 nM Cl-IB-MECA, was inhibited by increasing concentrations of MRE 3008F20 (0.3-30 nM) with an IC₅₀ of 0.90±0.08 nM (FIG. 13C-D).

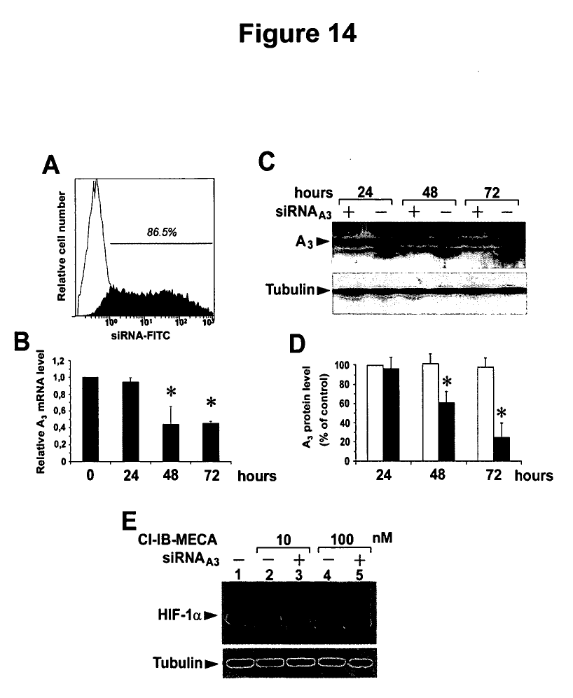

Finally, to further demonstrate that A₃ receptor is required for HIF-1α protein accumulation in response to adenosine, A375 cells were mock transfected or transfected with small interfering RNAs that target A₃ receptor mRNA (siRNA_{A3}) for degradation. To evaluate transfection efficiency A375 cells were also transfected with a siRNA control labeled with fluorescein. By flow cytometry we oberved a transfection efficiency of 85±5% (FIG. 7A). After transfection, the cells were cultured in complete media and at 24, 48 and 72 hours total RNA was isolated for Real-Time RT-PCR analysis of A₃ receptor mRNA and for Western blot analysis of A₃ receptor protein. As expected, A₃ receptor mRNA levels were significantly reduced in cells transfected with siRNA_{A3} (FIG. 14B). Furthermore, A₃ receptor protein expression was strongly reduced in siRNA_{A3}-treated cells (FIG. 14C-D). Neither mock transfection nor transfection with an siRNA targeted to an irrelevant mRNA inhibited A₃ receptor mRNA or protein expression. Therefore, at 72 hours from the siRNA_{A3} transfection, A375 cells were exposed to increasing concentrations of the A₃ adenosine receptor agonist Cl-IB-MECA (1-100 nM) for 4 hours in hypoxia. Total protein were then harvested for Western blot analysis. As control, A375 cells were exposed to RNAiFect™ Transfection reagent without siRNA_{A3}. We found that the inhibition of A₃ receptor expression is sufficient to block Cl-IB-MECA-induced HIF-1α accumulation (FIG. 14E).

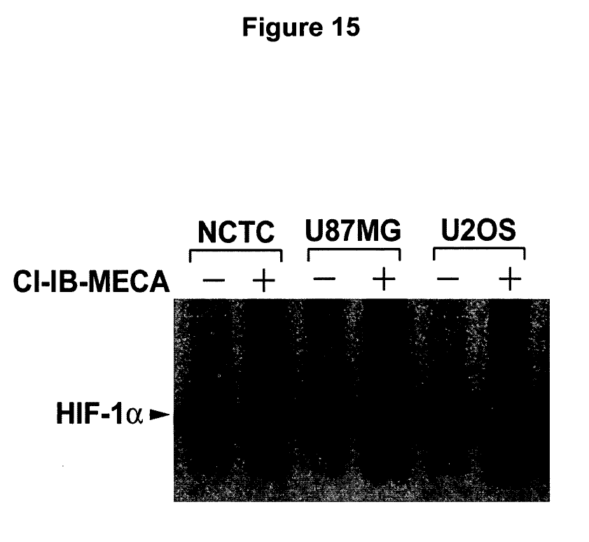

To determine whether the effect of A₃ receptor stimulation on HIF-1α expression was a general phenomenon, we assessed the ability of Cl-IB-MECA to induce HIF-1α levels in a variety of cell lines expressing A₃ adenosine receptors. After 4 hours of hypoxia under Cl-IB-MECA treatment, we were able to detect a significant increase in HIF-1α protein expression, both in human keratinocytes NCTC 2544 and in different human tumor U87MG glioblastoma and U2OS osteosarcoma cells (FIG. 15).

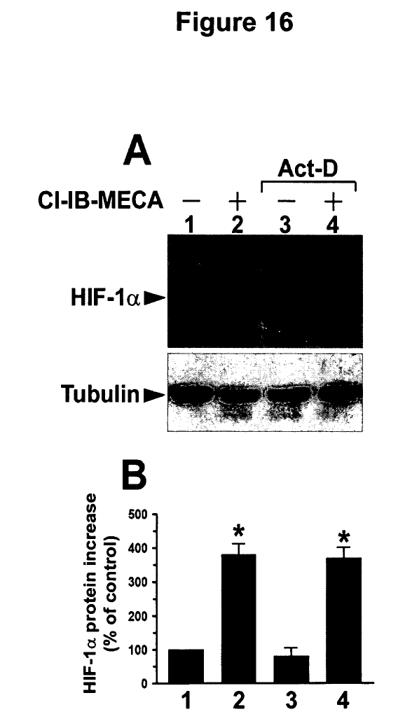

A₃ receptor mediates HIF-1α accumulation through a transcription-independent and translation-dependent pathway. To obtain a better understanding of the processes involved in HIF-1α accumulation in response to A₃ receptor stimulation in hypoxia, we investigated the effect of Cl-IB-MECA on the HIF-1α mRNA accumulation. After a treatment of A375 cells for 4 hours in hypoxia, RNA was extracted, and Real-Time RT-PCR analysis was performed. Activation of melanoma cells with 10 nM, 100 nM and 1 μM Cl-IB-MECA produced, respectively, a 1.13±0.10, 1.25±0.15 and 1.19±0.13 fold increase of HIF-1α mRNA accumulation with respect to the corresponding untreated cells, suggesting that A₃ receptor stimulation does not regulate HIF-1α mRNA transcription. To confirm this hypothesis, A375 cells were pretreated with 10 μg/ml actinomycin D (Act-D) to inhibit de novo gene transcription. Then, A375 cells were cultured for 4 hours in hypoxia in the presence of increasing concentrations of Cl-IB-MECA (100 nM). We found that A₃ receptor stimulation was able to increase HIF-1α protein expression also in the presence of Act-D (FIG. 16).

HIF-1α has been shown to be degraded through the proteasome pathway during normoxia. The enzymatic hydroxylation of proline 564 of HIF-1α controls the turnover of the protein by tagging it for interaction with the von Hippel Lindau protein (Ivan 2001; Jaakkola, 2001; Yu 2001; Maxwell 1999). When cells are hypoxic, the proline residue is not hydroxylated and HIF-1α protein accumulates. The effect of hypoxia on Pro-564 hydroxylation can be mimicked by transition metals like cobalt, iron chelators and by inhibitors of the prolyl hydroxylase enzymes (Ivan et al., 2001; Jaakkola et al., 2001).

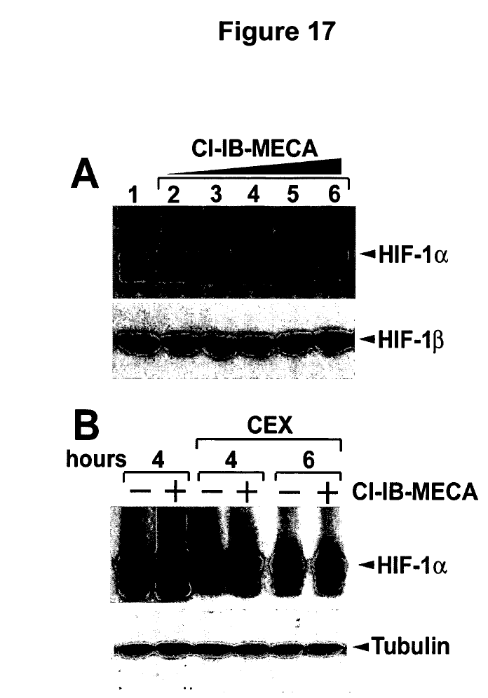

We tested the ability of A₃ adenosine receptor to modulate HIF-1α accumulation in the presence of the prolyl hydroxylase enzymes inhibitor, cobalt chloride (CoCl₂). We observed that A₃ receptor stimulation was able to increase the levels of HIF-1α protein also in CoCl₂-treated cells (FIG. 17A). To determine if A₃ receptor induces HIF-1α expression through a translation-dependent pathway, we determined HIF-1α protein modulation in the presence of the protein translation inhibitor, cycloheximide (CHX). To do this, A375 cells were cultured in normoxia for 4 hours in the presence of 100 μM CoCl₂, preventing oxygen-dependent HIF-1α protein degradation, and then A375 cells were treated with 100 nM Cl-IB-MECA in the presence or absence of CHX (1 μM). In cells exposed to CHX, Cl-IB-MECA failed to increase HIF-1α levels within 6 hours, as observed in the absence of CHX (FIG. 17B). Together, these results suggest that A₃ receptor activation increases HIF-1α protein levels through a translation-dependent pathway.

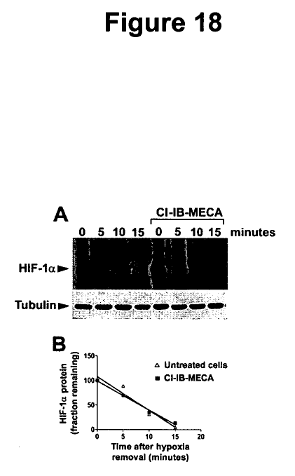

After return of hypoxic A375 cultures to normoxia the levels of HIF-1α protein decreased very rapidly and were abrogated after 15 minutes (FIG. 18A). Therefore, to study the effect of A₃ receptor activation on HIF-1α degradation, A375 cells were incubated in hypoxia in the absence and in the presence of Cl-IB-MECA 100 nM. After 4 hours, melanoma cells were exposed to normoxia and a time-course of HIF-1α disappearance was performed. Within 15 minutes after the removal of hypoxic conditions, a decrease in HIF-1α protein could be seen, in the absence and in the presence of Cl-IB-MECA with unchanged degradation rate (FIG. 18B). These results indicate that A₃ receptor activation is not able to prevent HIF-1α degradation in normoxic conditions.

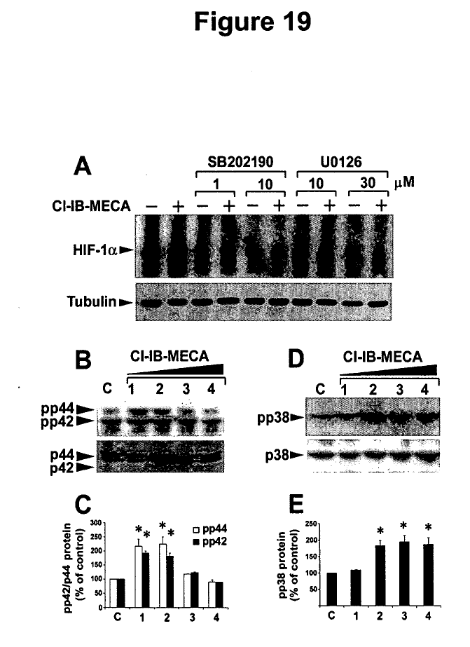

The main intracellular signaling pathways sustained by A₃ receptors during HIF-1α accumulation in hypoxia. It has been demonstrated that MAPK are involved in HIF-1α activation. To determine whether MAPK pathway was required for HIF-1α protein increase induced by A₃ receptor activation, A375 cells were pretreated with U0126, which is a potent inhibitor of MEK1/2, an upstream regulator of the phosphorylation of p44/p42 (Favata 1998), or with the inhibitor of p38 MAPK, SB202190 (Kramer 1996). Cells were then exposed to Cl-IB-MECA 100 nM for 4 hours in hypoxia, and total cellular protein extracts were prepared for immunoblot assay of HIF-1α and tubulin protein levels. As shown in FIG. 19A, both MEK inhibitor, U0126 (10 and 30 μM), and p38 MAPK inhibitor, SB202190 (1 and 10 μM), were able to inhibit Cl-IB-MECA-induced increase of HIF-1α protein expression. These results suggest that p44/p42 and p38 MAPK activity were required for the HIF-1α expression increase induced by A₃ receptor activation.

Furthermore, to confirm that p44/p42 and p38 MAPK belong to the signaling pathways fired by A₃ receptor stimulation, we also investigated endogenous p44/p42 and p38 MAPK activation levels in response to A₃ receptor agonist treatment. A375 cells were incubated with increasing concentrations of Cl-IB-MECA (1-1000 nM) for 4 hours in hypoxia, and the total cellular protein extracts were used to determine levels of phospho-p44, phospho-p42, and phospho-p38.

As shown in FIG. 19B, phosphorylation of p44 and p42 was induced in response to nanomolar concentrations of Cl-IB-MECA and the induction of p44/p42 kinases phosphorylation status was maximum by the treatment of A₃ receptor agonist 100 nM (FIG. 19C). Furthermore, we have monitored the activation levels of p38 MAPK upon A₃ receptor stimulation by the detection of its phosphorylated form on Western blot. As can be seen in FIG. 19D, a strong increase in the phosphorylation of p38 MAPK was observed after 4 hours of A₃ receptor stimulation in hypoxia. In particular, exposure of A375 cells to various concentrations of Cl-IB-MECA increased the phosphorylation of p38 MAPK in a dose-dependent manner (FIG. 19E). Phospho-p44, phospho-p42 and phospho-p38 blots were then stripped and reblotted with an antibody that equally recognizes total p44, p42 and p38 MAPKs. We found that the observed changes in phosphorylation level of p44, p42 and p38 MAPKs were not accompanied by a significative modulation in the expression levels of total proteins (FIG. 19 B-D).

DISCUSSION: To our knowledge, this is the first report which describes the role of adenosine in modulating the cellular response during hypoxia in an O₂-sensitive cell.

Hypoxia represents one of the first events in the growth of the cancer; this process creates conditions that, on one hand, are conducive to the accumulation of extracellular adenosine and, on the other hand, stabilize hypoxia-inducible factors, such as HIF-1α (Winn, 1981; Decking 1997; Ledoux 2003).

The results of the present study indicate a new way by which hypoxia may contribute to cancer development, based on the natural pathways of adenosine receptor-mediated signaling. For the first time, here we demonstrate that adenosine is able to increase HIF-1α protein expression in response to hypoxia in a dose- and time-dependent manner in A375 human melanoma cells, whereas HIF-1β protein levels are not affected.

We have previously demonstrated that all four adenosine receptors are expressed in human melanoma A375 cells (Merighi, 2001). Here, we report that A₃ receptor subtype mediates the observed adenosine effects on HIF-1α regulation in this cell line.

The effects of adenosine on HIF-1α protein accumulation are not mediated by A₁, A_{2A} or A_{2B} receptors. In support of this conclusion, DPCPX, SCH 58261 and MRE 2029F20, adenosine receptor antagonists highly selective for A₁, A_{2A} and A_{2B} receptors, respectively, did not block the stimulatory effect of adenosine on HIF-1α protein increase.

The conclusion that the effects of adenosine on HIF-1α accumulation are mediated via A₃ receptors is supported by the observation that the stimulatory effects of this nucleoside on HIF-1α protein are mimicked by the A₃ receptor agonist Cl-IB-MECA and inhibited by A₃ receptor antagonists, MRE 3008F20 and MRE 3005F20. In particular, the potencies of these drugs are in agreement with their inhibitory equilibrium binding constant (Ki) observed in binding experiments for the adenosine A₃ receptor (Merighi 2001).

Furthermore, the inhibition of A₃ receptor expression at the mRNA and protein level is sufficient to block A₃ receptor-induced HIF-1α protein accumulation. Therefore, our results indicate that the cell surface A₃ adenosine receptor transduces extracellular hypoxic signals into the cell interior. A₃ receptors are present in melanoma cells and their expression appears to be a bridge between the hypoxic insult and HIF-1α accumulation, regulating the cellular response to hypoxia, like an oxygen-sensing receptor. The extent to which A₃ receptor influences the ability of tumor cells to respond to hypoxia will require further investigation.

Similar results obtained in different cells (keratinocytes, melanoma, osteosarcoma, glioblastoma) raised the concern that the A₃ receptor stimulation effect on HIF-1α protein expression in hypoxia may be indiscriminate between normal and cancer cells, thereby demonstrating that this may be a general signaling pathway shared by many, if not all, cell types.

A₃ adenosine receptor stimulation had no effect on HIF-1α mRNA accumulation, as observed by Real-Time RT-PCR experiments. Accordingly, Act-D experiments indicate that A₃ receptor does not regulate HIF-1α protein expression through a transcription-dependent mechanism. The lack of adenosine effect on HIF-1α at transcriptional level is not surprising in view of the fact that hypoxic regulation of HIF-1α is primarily determined by stabilization of HIF-1α protein (Huang, 1998). In addition, we have obtained evidences that A₃ adenosine receptors modulate HIF-1α protein levels through a translation-dependent pathway while did not affect HIF-1α oxygen-dependent degradation. Our data suggest that A₃ adenosine receptors does not increase the half-life of HIF-1α protein while may increase the rate of HIF-1α protein synthesis, in a manner similar to the effect of various growth factors (Zhong 2000; Fukuda, 2002). Nevertheless, we cannot exclude the possibility that A₃ adenosine receptor regulates the translation of a protein, which inhibits HIF-1α degradation.

Phosphorylation and dephosphorylation activities have been suggested to be critical in the signaling pathway leading to HIF-1 activation. Several reports demonstrated that hypoxia induces the phosphorylation of HIF-1α by p44/p42 and p38 MAPKs, which increases both HIF-1α nuclear localization and transcriptional activity (Semenza 2001CurrOpCB; Richard 1999BBRC; Berra 2000; Richard 1999JBC; Conrad 1999; Sodhi 2000; Mottet D, 2003; Semenza 2002). In addition, adenosine has been shown to directly enhance MAPK activity in A375 human melanoma cells (Merighi et al., 2002) but also in non human cell lines stably transfected with the human A₃ receptor (Hammarberg 2004; Schulte 2000-2002-2003). In the present study, we observed that p44/p42 and p38 MAPKs are necessary to increase HIF-1α levels but also that these kinases are included in the molecular signaling pathways generated by A₃ receptor engagement. In conclusion, the present study demonstrates that adenosine, via A₃ receptors, is able to increase the levels of HIF-1α through p44/p42 and p38 MAPK pathways. Actually, further studies are needed to evaluate the role of p44/p42 and p38 MAPK in the reduced turnover, increased life and transduction of HIF-1α protein in hypoxia.

HIF-1α is overexpressed in tumors as a result of hypoxia and is involved in key aspects of tumor biology, such as angiogenesis, invasion and altered energy metabolism (Ratcliffe 2000). It is recognized that the inhibition of HIF-1 activity represents a novel therapeutic approach to cancer therapy, especially in combination with angiogenesis inhibitors, which would further increase intratumoral hypoxia and thus provide an even greater therapeutic window for use of an HIF-inhibitor. Recent studies indicate that pharmacologic inhibition of HIF-1α and particularly of HIF-regulated genes that are important for cancer cell survival may be more advantageous than HIF-gene inactivation therapeutic approaches (Mabjeesh et al., 2003). Many normal tissues function at pO₂ values sufficient to activate HIF, and the system has important functions under normal physiological conditions (Hopfl, 2004). This will need to be considered in the development of pharmacological inhibitors for clinical use.

Given the ability of A₃ adenosine receptor antagonists to block HIF-1α protein expression accumulation induced by adenosine, our data imply that A₃ adenosine receptor antagonists may be useful in cancer therapy. In particular, we remark that in in vivo system the extracellular fluid of solid tumors contains increased levels of adenosine (Blay et al., 1997), the endogenous agonist responsible for adenosine receptor functions. Therefore, with A₃ receptor antagonists in cancer therapy it may be possible to achieve tissue selectivity, such that a biological effect would only be observed in tumoral hypoxic cells, where high adenosine concentration increases HIF-1α accumulation.

Additional studies are warranted to determine whether A₃ receptor antagonists can block the survival of hypoxic solid tumors.

What is claimed is:

1. A method of treating cancer in a subject in need thereof, comprising administering an effective amount of an adenosine A₃ receptor antagonist, wherein the cancer is a HIF-1α expressing cancer.

2. The method of claim 1, wherein the cancer is selected from the group consisting of cervical cancer, lung cancer, breast cancer, oligodendroglioma, orpharyngeal squamous cell carcinoma, ovarian cancer, oesophageal cancer, endometrial cancer, head and neck cancer, human lung carcinoma, human colon carcinoma, pancreatic cancer, prostate cancer and gastrointestinal stromal tumor of the stomach.

3. The method of claim 1, wherein the cancer is further an A₃ receptor expressing cancer.

4. The method of claim 1, further comprising the administration of one or more additional cancer therapies.

5. The method of claim 4, wherein said additional cancer therapy is selected from the group consisting of chemotherapy, immunotherapy, radiation therapy, hormonal therapy, or surgery.

6. The method of claim 1, further comprising administering one or more anti-cancer agents.

7. The method of claim 6, wherein the anti-cancer agent is selected from the group consisting of a chemotherapeutic agent, an anti-angiogenic agent, a cytotoxic agent and a cancer therapeutic antibody.

8. The method of claim 1, wherein the A₃ receptor antagonist is a compound or pharmaceutically acceptable salt of the compound having the following general formulas: 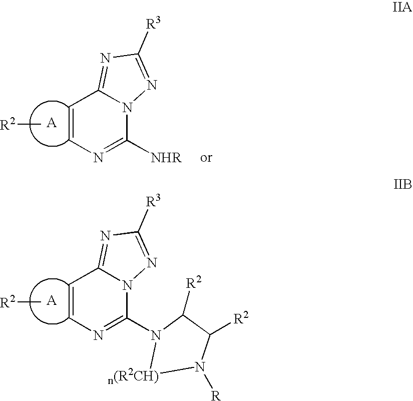
wherein:
A is imidazole, pyrazole, or triazole;
R is —C(X)R¹, —C(X)—N(R¹)₂, —C(X)OR¹, —C(X)SR¹, —SO_{b}R¹, —SO_{b}OR¹, —SO_{b}SR¹, or SO_{b}—N(R¹)₂;
R¹ is hydrogen, alkyl, substituted alkyl, alkenyl, substituted alkenyl, alkynyl, substituted alkynyl, aryl, substituted aryl, heteroaryl, substituted heteroaryl, heterocyclic, substituted heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms such as N, O, S;
R² is hydrogen, alkyl, alkenyl, alkynyl, substituted alkyl, substituted alkenyl, substituted alkynyl, aralkyl, substituted aralkyl, aryl, substituted aryl, heteroaryl, or substituted heteroaryl;
R³ is furan, pyrrole, thiophene, benzofuran, indole, benzothiophene, optionally substituted with one or more substituents as described herein for substituted heteroaryl rings;
X is O, S, or NR¹; and
b is 1 or 2;
or,
the A₃ receptor antagonist is a compound or pharmaceutically acceptable salt of the compound having the following general formula: 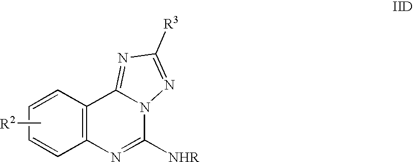
wherein:
R is —C(X)R¹, —C(X)—N(R¹)₂, —C(X)OR¹, —C(X)SR¹, —SO_{b}R¹, —SO_{b}OR¹, —SO_{b}SR¹, or —SO_{b}N(R¹)₂;
R¹ is hydrogen, alkyl, substituted alkyl, alkenyl, substituted alkenyl, alkynyl, substituted alkynyl, aryl, heteroaryl, heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms such as N, O, S;
R² is hydrogen, halogen, preferably chloro, alkyl, alkenyl, alkynyl, substituted alkyl, substituted alkenyl, substituted alkynyl, aralkyl, substituted aralkyl, aryl, substituted aryl, heteroaryl or substituted heteroaryl;
R³ is furan, pyrrole, thiophene, benzofuran, indole, benzothiophene, optionally substituted with one or more substituents as described herein for substituted heteroaryl rings;
X is O, S, or NR¹; and
b is 1 or 2.

9. The method of claim 8, wherein the A₃ receptor antagonist is a compound or pharmaceutically acceptable salt of the compound having the general formula IIA:
wherein:
A is imidazole, pyrazole, or triazole;
R is —C(X)R¹, —C(X)—N(R¹)₂, —C(X)OR¹, —C(X)SR¹, —SO_{b}R¹, —SO_{b}OR¹, —SO_{b}SR¹, or SO_{b}—N(R¹)₂;
R¹ is hydrogen, alkyl, substituted alkyl, alkenyl, substituted alkenyl, alkynyl, substituted alkynyl, aryl, substituted aryl, heteroaryl, substituted heteroaryl, heterocyclic, substituted heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms such as N, O, S;
R² is hydrogen, alkyl, alkenyl, alkynyl, substituted alkyl, substituted alkenyl, substituted alkynyl, aralkyl, substituted aralkyl, aryl, substituted aryl, heteroaryl, or substituted heteroaryl;
R³ is furan, pyrrole, thiophene, benzofuran, indole, benzothiophene, optionally substituted with one or more substituents as described herein for substituted heteroaryl rings;
X is O, S, or NR¹; and
b is 1 or 2.

10. The method of claim 9, wherein the A₃ receptor antagonist is a compound or pharmaceutically acceptable salt of the compound wherein:
A is pyrazole;
R is —C(X)R¹, —C(X)—N(R¹)₂, —C(X)OR¹, —C(X)SR¹, —SO_{b}R¹, —SO_{b}OR¹, —SO_{b}SR¹, or SO_{b}—N(R¹)₂;
R¹ is hydrogen, alkyl, substituted alkyl, alkenyl, substituted alkenyl, alkynyl, substituted alkynyl, aryl, substituted aryl, heteroaryl, substituted heteroaryl, heterocyclic, substituted heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms such as N, O, S;
R² is hydrogen, alkyl, alkenyl, alkynyl, substituted alkyl, substituted alkenyl, substituted alkynyl, aralkyl, substituted aralkyl, aryl, substituted aryl, heteroaryl, or substituted heteroaryl;
R³ is furan;
X is O, S, or NR¹; and
b is 1 or 2.

11. The method of claim 10, wherein the A₃ receptor antagonist is a compound or pharmaceutically acceptable salt of the compound wherein:
A is pyrazole;
R is —C(O)R¹, —C(O)—N(R¹)₂, —C(O)OR¹, or —C(O)SR¹;
R¹ is hydrogen, alkyl, substituted alkyl, alkenyl, substituted alkenyl, alkynyl, substituted alkynyl, aryl, substituted aryl, heteroaryl, substituted heteroaryl, heterocyclic, substituted heterocyclic, lower alkenyl, lower alkanoyl, or, if linked to a nitrogen atom, then taken together with the nitrogen atom, forms an azetidine ring or a 5-6 membered heterocyclic ring containing one or more heteroatoms such as N, O, S;
R² is hydrogen, alkyl alkenyl, alkynyl, substituted alkyl substituted alkenyl, substituted alkynyl, aralkyl, substituted aralkyl, aryl, substituted aryl, heteroaryl, or substituted heteroaryl; and
R³ is furan.

12. The method of claim 11, wherein the A₃ receptor antagonist is a compound or pharmaceutically acceptable salt of the compound having the general formula IIE 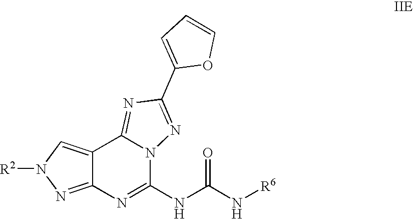
wherein R² is alkyl and
R⁶ is aryl, substituted aryl, heteroaryl or substituted heteroaryl.

13. The method of claim 12, wherein the A₃ receptor antagonist is the compound or pharmaceutically acceptable salt having one of the following structural formulas:

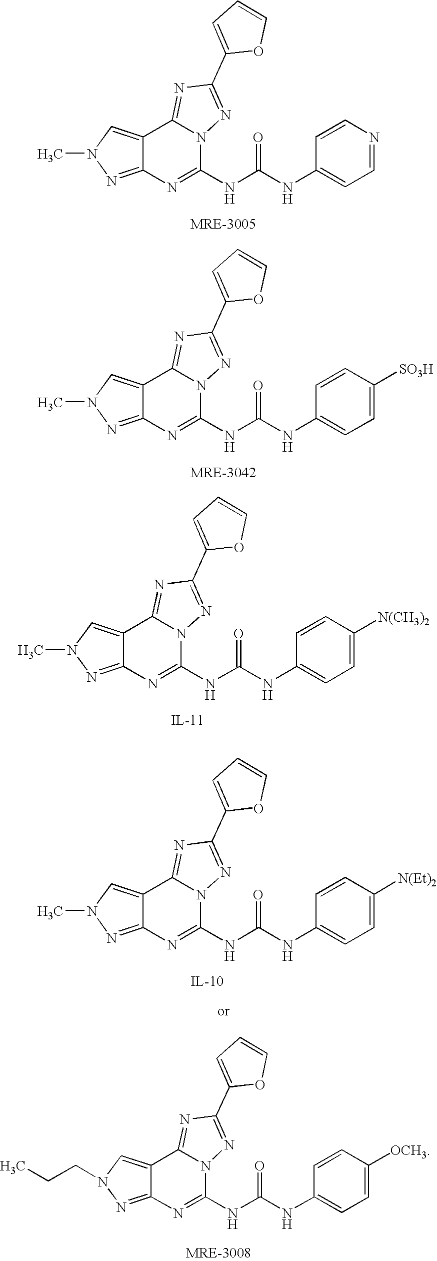

14. A method of inhibiting the growth of a solid hypoxic tumor in a subject, comprising administering an effective amount of an adenosine A₃ receptor antagonist.

15. The method of claim 14, wherein the growth of the solid hypoxic tumor is reduced by at least 10%.

16. The method of claim 14, wherein the growth of the solid hypoxic tumor is reduced by at least 30%.

17. The method of claim 14, wherein the growth of the solid hypoxic tumor is reduced by at least 60%.

18. A method for treating an HIF-1-mediated disorder in a subject comprising administering an effective amount of an adenosine A₃ receptor antagonist.

19. The method of claim 18, wherein the adenosine A₃ receptor antagonist reduces the level of expression of HIF-1α.

20. The method of claim 18, wherein the level of expression of HIF-1α is reduced by at least 10%.

21. The method of claim 18, wherein the HIF-1 mediated disorder is selected from the group consisting of cancer, chronic obstructive pulmonary disease, and asthma.

22. The method of claim 1, 14 or 18, further comprising the steps of (a) removing a tissue sample from the subject; and (b) measuring a HIF-1α level using a HIF-1α probe.

23. The method of claim 22, wherein the HIF-1α probe is selected from the group consisting of an antibody, an antigen, a nucleic acid, a protein, and a small molecule.

24. The method of claim 23, wherein the antibody is a monoclonal antibody.

25. The method of claim 24, wherein the antibody immunospecifically binds HIF-1α or a fragment or derivative thereof.

26. The method of claim 22, wherein the step of measuring a level of HIF-1α comprises testing at least one aliquot, said step of testing comprising:
(a) contacting the aliquot with an antibody that is immunospecific for HIF-1α, and
(b) detecting whether binding has occurred between the antibody and at least one species in the aliquot.

27. The method of claim 22, wherein the step of measuring a level of HIF-1α comprises testing at least one aliquot, said step of testing comprising:
(a) contacting the aliquots with an isolated nucleic acid that is hybridizable to the nucleic acid that encode HIF-1α, and
(b) detecting whether hybridization has occurred between the nucleic acid probe and at least one species in the aliquot.

28. A method for inhibiting the progression of cancer, in a subject, by administering a prophylactically effective amount of an A₃ receptor antagonist, wherein the cancer is characterized by an overexpression of HIF-1α.
